(19) United States
(12) Patent Application Publication
(10) Pub. No.: US 2022/0341951 A1
(43) Pub. Date: Oct. 27, 2022

(54) SYSTEMS AND METHODS FOR MENSTRUAL CYCLE TESTING

(71) Applicant: MFB Fertility, Inc., Boulder, CO (US)

(72) Inventors: Amy BECKLEY, Erie, CO (US); Jeffrey SCHELL, Denver, CO (US); Ellen SCHELL, Denver, CO (US)

(73) Assignee: MFB Fertility, Inc., Boulder, CO (US)

(21) Appl. No.: 17/636,755

(22) PCT Filed: Jul. 2, 2020
(86) PCT No.: PCT/US2020/040600

§ 371 Date: Feb. 18, 2022

Related U.S. Application Data
(63) Continuation-in-part of application No. 16/732,766, filed on Jan. 2, 2020, now Pat. No. 11,029,321.
Application No. 16/732,766 is a continuation-in-part of application No. 16/544,554, filed on Aug. 19, 2019.
Application No. 16/732,766 is a continuation-in-part of application No. 15/974,229, filed on May 8, 2018.
Application No. 16/732,766 is a continuation-in-part of application No. 15/900,794, filed on Feb. 20, 2018, now abandoned.
Application No. 16/732,766 is a continuation-in-part of application No. 16/381,229, filed on Apr. 11, 2019.
Application No. PCT/US2020/040600 is a continuation-in-part of application No. 16/732,823, filed on Jan. 2, 2020, now Pat. No. 11,061,026.
Application No. 16/732,823 is a continuation-in-part of application No. 16/544,554, filed on Aug. 19, 2019.
Application No. 16/732,823 is a continuation-in-part of application No. 15/974,229, filed on May 8, 2018.
Application No. 15/974,229 is a continuation-in-part of application No. 15/900,794, filed on Feb. 20, 2018, now abandoned.
Application No. 16/732,823 is a continuation-in-part of application No. 16/381,229, filed on Apr. 11, 2019.
(60) Provisional application No. 62/720,953, filed on Aug. 22, 2018.
(60) Provisional application No. 62/503,223, filed on May 8, 2017.
(60) Provisional application No. 62/460,307, filed on Feb. 17, 2017.
(60) Provisional application No. 62/611,467, filed on Dec. 28, 2017.
(60) Provisional application No. 62/720,953, filed on Aug. 22, 2018.
(60) Provisional application No. 62/503,223, filed on May 8, 2017.
(60) Provisional application No. 62/460,307, filed on Feb. 17, 2017.
(60) Provisional application No. 62/611,467, filed on Dec. 28, 2017.

(51) Int. Cl. G01N 33/76; A61B 10/00; G16H 10/60; G01N 33/52

(52) U.S. Cl. CPC G01N 33/76; A61B 10/0012; G16H 10/60; G01N 33/52; A61B 2010/0006

(57) ABSTRACT

The present invention relates to a system and methods for evaluating and tracking the operation of the menstrual cycle and treating undesirable trends associated with the menstrual cycle. Various aspects of the system and methods described herein rely upon the operation of diagnostic tests specially configured to evaluate a bodily fluid for the presence or absence of hormones or analytes, and more specifically configured to evaluate a bodily fluid for at least the presence or absence of pregnanediol glucuronide at a threshold selected from the range inclusive of 1 μg/mL-10 μg/mL. The results from one or more diagnostic tests are interpreted in accordance with the teachings of the system. The interpretations are useful in accordance with facilitating treatments associated with medical conditions correlated to the generated interpretations, optionally delivered during a consultation with a medical provider during a telemedicine consultation, the treatments optionally comprising dietary changes incorporating the consumption of certain seeds to mitigate hormonal imbalances.

### TECHNICAL FIELD

The present invention relates to diagnostic testing associated with the female reproductive system, and more specifically, testing associated with the menstrual cycle.

### BACKGROUND ART

Many changes result from the change in hormones that occurs in conjunction with menstrual cycles. Such changes often result in hormone imbalances which can impact on a woman's life. For instance, when trying to conceive, suboptimal hormonal concentrations can undesirably make conception difficult or impossible. During puberty, hormonal changes can result in unfamiliar changes in mood and physical changes. During menopause, hormonal imbalances may cause a number of adverse side effects, including hot flashes, night sweats, osteoporosis, insomnia, vaginal dryness, pain associated with sexual intercourse, weight gain, lack of interest in sex and mood swings. Therefore, having precise and updated knowledge of hormonal concentrations is desirable for many women.

Hormone change patterns associated with the above symptoms may be detectable if women have the capacity to test on a repetitive and sufficiently simplified basis. However, existing testing protocols are generally difficult to track, require at least partial serum testing or laboratory work, and deliver results inappropriate for many lay users. Existing testing paradigms, including serum tests, at-home pregnancy and ovulation predictor kits (OPKs) configured to evaluate urine for luteinizing hormone (LH), are often technical in nature and difficult for lay users to interpret. Moreover, due in part to the sporadic timeframes for testing hormones at different times during a menstrual cycle or otherwise, trend identification associated with hormonal concentrations is often difficult or impossible. Unfortunately, suboptimal hormonal patterns may result in additional difficulty with attempts to conceive or infertility.

Moreover, may prior art mechanisms associated with the detection of hormones or hormone metabolites provide only a limited set of information or a difficult to understand interpretation. As a result, those utilizing such mechanisms have difficulty acting upon the indications. For example, some diagnostic tests provide only a line, series of lines or cross to indicate the presence of a hormone or analyte. In many circumstances, very limited information is provided with regard to what the line, series of lines or cross indicates other than information associated with the presence of the relevant hormone or analyte, sometimes with a “high” or “low” indication, or other times by depicting a ‘smile’ face or ‘sad’ face. It is often confusing and a problem for the subject of a diagnostic test to need to refer to documentation separate from the indication of the test to retrieve an interpretation. It remains an unmet need to provide clearer interpretations of what each result on a diagnostic test indicates directly in association with a test result, particularly when optimal use of such a test occurs as part of a series of diagnostic tests and/or a plurality of diagnostic tests configured to detect the presence of more than one hormone and/or analyte over a period of time, especially in association with providing information associated with the menstrual cycle, puberty, fertility status and/or menopause.

Hormonal changes and hormonal pattern changes are frequently caused by menopause. Each day, tens of thousands of women around the world reach menopause. However, the associated transition can be challenging and last for many years. In fact, more than 80% of women report perimenopause symptoms as “debilitating”, impacting everything from work to social relationships to their sex lives. In some countries, the majority of women report being turned away by doctors who simply don't know how to help.

Most adverse symptoms associated with menopause result from the change in hormones that occurs as women transition out of ovulatory cycles. Such changes often result in hormone imbalances which can cause negative impacts on a woman's quality of life. These negative impacts may include hot flashes, night sweats, osteoporosis, insomnia, vaginal dryness, pain associated with sexual intercourse, weight gain, lack of interest in sex and mood swings.

When detected, the negative impacts may be partially or fully reversed with the assistance of hormonal treatments. However, the detection of single point hormone imbalances or trends of hormonal imbalances is often too expensive or inconvenient to be practical. Moreover, treatment regimens often need to change as the change in hormonal concentrations evolves over time, requiring repeated, ongoing testing paradigms. Thus, a supplement or treatment that worked for several months may suddenly cease having the desired effect as new hormonal pattern evolves with age, fertility status, diet, disease or otherwise in association with a particular woman. Thus, a cost-effective, continuous testing system to continually evaluate hormonal levels and suggest treatment protocols remains unsolved and is desirable.

Tracking hormonal changes provides information relevant to menopause, puberty and fertility as the menstrual cycle is controlled by the anterior pituitary gonadotropins follicle-stimulating hormone (FSH) and luteinizing hormone (LH) and the gonadal sex hormones estradiol and progesterone. Estrone-3-glucuronide (commonly abbreviated as E1G or E3G) is a principal metabolite of estradiol. Urinary levels of E3G correspond to the serum levels of estradiol. Similarly, pregnanediol glucuronide (PdG) is a principal metabolite of progesterone. The urinary levels of pregnanediol glucuronide correspond to the serum levels of progesterone.

The presence of follicle stimulating hormone is associated with the start of the menstrual cycle, as FSH stimulates growth of a follicle or follicles. The growing follicle triggers estrogen production. Increased estrogen from a growing follicle then triggers a sudden spike in LH. The surge in LH causes the follicle to rupture, which is ovulation. The ruptured follicle then secretes progesterone. Progesterone acts to thicken the uterine wall to prepare for implantation and protect the growing fetus. Once a fertilized embryo has implanted in the uterine wall, human chorionic gonadotropin (hCG) is released and is detectable in urine within a short period of time.

Various different at home urine based hormone tests are currently readily available to consumers to help track the hormones involved in the female menstrual cycle, including tests for FSH, LH and hCG. There are other at home tests that include E3 and LH together to give a digital reading to indicate low, high or peak fertility. However, there remains a need for a urine based at-home test that can provide the consumer information regarding their progesterone levels by detecting and even quantitating PdG. Notably, however, prior art attempts to discover PdG in urine have been plagued by many well-known problems adversely impacting the ability for laypersons to collect and understand the results of such attempts. Such attempts have proven fruitless due to inaccuracies associated with the tests with regard to the precision of detection and measurement of PdG in urine. Further, the proper chemistry to enable the creation of such a test has remained to be discovered in the prior art. Specifically, it has yet to have been discovered how to create a consistently reproducible test configured to detect PdG visible to the naked eye despite years of effort.

To create a test allowing one to visually review the results with the naked eye and without the assistance of lab-grade equipment, an alternative solution is needed. Previous attempts to create a lateral flow assay for detecting progesterone metabolites in urine, including the inventive matter disclosed in U.S. Pat. No. 6,924,153 granted on Aug. 2, 2005, the inventive matter disclosed in United Kingdom Patent Application Publication No. GB 2,204,398 A as published on Nov. 9, 1988, and similar prior art items, were unsuccessful due to the technical difficulties and inappropriate selection of component antibodies (namely the selection of component antibodies of improper isotypes) and type of carrier proteins. In certain cases, such difficulties also were associated with the development antigen and antibody chemistries of such ratios, component parts and/or elements to specifically produce visual results readable to the naked eye. Other prior art matter, for instance the subject matter disclosed in PCT/FR2016/050506 published on Mar. 4, 2016, and PCT/US2019/038173 filed on Jun. 19, 2019 only disclose Bovine Serum Albumin (BSA) as the carrier protein, which is a commonly used carrier protein and often inadequate for usage in a urine-based progesterone or PdG testing solution intended to display results visible and discernable to the naked eye, and also difficult to reliably reproduce at scale in association with the previously known manufacturing processes, and evidence other deficiencies such as the need to utilize external equipment to read results. Among other challenges associated with the solution disclosed in PCT/FR2016/050506, its disclosure of BSA as the carrier protein results in a testing solution lacking the ability to adequately bind to colloidal gold, thereby resulting in a test delivering results that are problematically imperceptible to the naked eye to the necessary usable perception level. Moreover, these and other prior art solutions have failed to produce a product that reliably and reproducibly produced enough color intensity to deliver clear and easily interpreted test results to users with minimal training and a lack of specialized equipment. Therefore, a need remains for a lateral flow assay for detecting progesterone metabolites in urine that reliably and reproducibly delivers enough color intensity to portray clear and easily interpreted test results to users with minimal training and a lack of specialized equipment.

Prior art attempts to provide a test sufficient to discover PdG in urine are associated with challenges stemming from problematic antibody selection and incorporation, often due to the selection and incorporation of improperly chosen antibodies and antibody isotypes, which may lead to the inappropriate choice of threshold and threshold ranges for testing. In some instances, it remains impossible to create and/or maintain a consistent threshold due to the chemistry of the attempted test. For instance, variance of testing threshold is likely to occur between and among lots of tests manufactured to detect the presence or absence of PdG at the threshold, particularly in association with previously unsuccessful or only partially successful attempts at creating PdG detection tests comprising BSA (conjugated to PdG) as the carrier protein. Previous attempts to create PdG and other tests configured to detect LH and hCG have been configured to incorporate thresholds that exceed the more appropriate thresholds relevant to testing hormones associated with the menstrual cycle. Such previous attempts, when manufactured at scale, exhibit variance in threshold from diagnostic test to diagnostic test due to their configuration, and in specific examples due to difficulties associated with utilization of BSA as the carrier protein, that make such tests impractical from which to derive information of value in association with a system to reliably provide indications relevant to the menstrual cycle or to a fertility status.

A common problem associated with prior art attempts is that the specifically chosen antibodies with such solutions are undesirably cross-reactive. In certain cases, chosen antibodies have suboptimal affinities for the application of a PdG test. The chosen antibodies in prior art attempts often fall outside of a desired threshold detection range. For instance, the chosen antibodies have resulted in an attempt at a diagnostic test that is not sensitive enough to allow a user to distinguish a positive and negative result. Sensitivity in such context may derive from suboptimal levels of affinity, avinity and specificity. In prior art tests where suboptimal sensitivity results from suboptimal specificity, the chosen antibody having a particular antibody isotype binds on items other than a PdG target. A problem with prior art attempted tests having a particular suboptimal combination antibody, antibody isotype and/or carrier protein, is that the antibody and the conjugate do not bind with the precision necessary to produce a viable, reproducible test result reliably useful to detect the presence of PdG at a consistent threshold chosen from within the range necessary to provide interpretations relevant to the menstrual cycle and/or fertility status. Therefore, it remains an unmet need and highly desirable to provide a lateral flow assay configured to detect the presence or absence of PdG in a bodily that can be consistently and reliably reproduced, optionally for utilization in association with a system associated with providing information relevant to the menstrual cycle and/or fertility status.

Diagnostic tests also vary in terms of the correlation of a positive or negative result to the presence or absence of a line. For instance, most LH (ovulation prediction) and hCG (pregnancy) tests as known in the art provide two lines in association with a positive result. However, in association with testing for other hormones and/or hormonal analytes, the presence of only one line, or the absence of a line, may indicate a positive result. Therefore there is a need for an improved mechanism that optionally allows for the interpretation of the indicated results for laypersons in a less confusing manner, particularly in a case where multiple results are indicated on the same test for a single sample. It also remains preferable to provide an inexpensive or commonly available mechanism that is less complicated than relying exclusively upon uncommonly used external computing devices or computing devices provided directly within in a cartridge containing the diagnostic test.

Likewise, some prior art progesterone tests remain limited to use within a lab environment. Colorimetric lab-grade electronic readers are used to detect differences in color otherwise imperceptible to the naked eye. Such lab-based tests determine concentrations of progesterone in serum to a high accuracy, often with the assistance of lasers, but are inaccessible or impractical to most laypersons.

The consumption of certain seeds provides benefits in association with balancing hormones, boosting fertility, and easing symptoms of menopause. Traditionally understood mechanisms associated with seed consumption, also referred to as “seed cycling,” involve eating seeds at different points during the menstrual cycle to balance certain hormones. More specifically, it is understood that seed cycling is a naturopathic remedy that balances hormones by regulating the hormone estrogen in the first half of a woman's menstrual cycle and the hormone progesterone in the second half of a woman's menstrual cycle. However, a problem remains associated with the inconvenience of measuring a specific amount of a specific type of seed to consume in relation to seed cycling. A related problem is knowing precisely when to consume each specific type of seed to consume, especially in relation to a level of hormone present in the blood.

During the follicular phase, it has been demonstrated the phytoestrogens in flax seeds can help moderate estrogen levels. Phytoestrogens are compounds in plants that can mimic the action of estrogen. Additionally, zinc present in pumpkin seeds has been demonstrated to promote progesterone production. During the luteal phase, lignans, a type of polyphenol present in sesame seeds, may inhibit estrogen levels from increasing too much. Meanwhile, the vitamin E in sunflower seeds has been demonstrated to boost progesterone levels.

Despite the evidence linking the consumption of seeds in various points of the menstrual cycle to hormone regulation, there remains a need to trigger or prompt the specific day to consume a specific type seed to obtain the desired hormone balancing effect. More specifically, there remains a need to generate the trigger based on an accurate, reproducible diagnostic test confirming the start of a different phase in direct response to the detected presence or absence of a specific hormone or analyte in a specific subject woman. There remains a need, in association with such seed cycling, to have a readily consumable form of food containing a specified quantity of the specific type of seed to avoid the inconvenience associated with measuring such seeds. Due to the general lack of precise knowledge of when to consume a certain type of seed in response to a blood hormone level, it also remains desirable to have a prompt to consume a specific type of seed in a readily consumable form containing a specified quantity of the specific type of seed immediately following and in direct response to detected hormone levels at a specific time during a specific woman's menstrual cycle to improve the effectiveness and usefulness of seed cycling. An improved seed cycling system is therefore desirable to address such problems.

Due to the difficulty associated with the creation of an accurate, reproducible diagnostic test to detect PdG at a consistent threshold in urine, it has likewise been untenable in the prior art to have a system to read, record and track the results of such diagnostic tests configured to detect PdG, either alone or in conjunction with urine based tests configured to detect for the presence of other hormones or analytes, such as LH, FSH, an estrogen metabolite such as E3G and/or hCG. The measurement of progesterone, which in some instances can be estimated in accordance with a urine measurement of progesterone analyte PdG, and corresponding changes over time within a menstrual cycle, and corresponding trends from one menstrual cycle to the next, are crucial aspects associated with many important methods associated with tracking fertility, the menstrual cycle, menopause, or triggering the consumption of certain foods at specified timeframes during the menstrual cycle or the prescription of certain hormone supplementations for supplementation at specified timeframes during the menstrual cycle. It is impractical to identify the hormonal levels, particularly the levels of progesterone, via serum testing on a daily basis in association with such important methods given the logistical challenges (i.e. travel to a laboratory, waiting for evaluated results) involved. Therefore, the successful deployment or utilization of methods in association with tracking fertility, the menstrual cycle, menopause, or triggering the consumption of certain foods or the prescription of certain hormone supplementations has been limited in association with the lack of such accurate, reproducible diagnostic tests. Thus, it remains desirable and an unmet need to have a sufficient testing associated with the detection of the presence or absence of PdG in urine for use in association with a system configured to aid in fertility tracking, menopause tracking, pregnancy avoidance, menstrual cycle tracking and/or puberty tracking. Likewise, it remains desirable and a previously unmet need to have the capability, optionally in conjunction with an organized system, to engage in such diagnostic data collection activity in private due to the sensitivity of the information collection, with the conveniences associated with testing at home, without the hassle or cost of consulting with a physician or laboratory representative.

### Summary of Invention/Technical Problem

The present invention solves the previously existing inability to detect, organize and interpret a specific concentration of progesterone in blood or its corresponding analyte in urine, and other hormones and analytes related to the menstrual cycle, due to configurations of previously existing diagnostic tests and the unavailability of a system configured to detect and interpret the results of diagnostic tests.

### Solution to Problem

A variety of novel diagnostic tests have allowed for the creation of a system to track hormonal levels associated with the menstrual cycle without requiring the assistance of a laboratory. Such tests include diagnostic tests configured to evaluate an applied fluid to detect and provide an indication for the presence or absence of pregnanediol glucuronide at a threshold selected from the range inclusive of 0.1 μg/mL-20 μg/mL to provide usefulness for nearly all women, and more particularly 1 μg/mL-10 μg/mL to provide usefulness for the vast majority of women. Further, in coordination with technologies specially configured to interpret the results of such tests, format the results of such tests as unique messages and deliver the results of such tests to a specially defined subset of displays accessible to a layperson user, optionally the subject woman whose fluid has been evaluated by such tests, the solution in an example comprises mechanisms for testing bodily fluids and providing an immediate interpretation of the results of such tests to a layperson user, optionally for further delivery to a healthcare professional. In some circumstances, it remains desirable to discuss a result or a series of results obtained from such tests indicating an undesirable medical condition with a healthcare professional and obtain suggested treatment protocols. An aspect of the solution in an example described herein comprises a system for conducting a telemedicine consultation in such a manner that the collected results of such tests are made available to a medical provider for discussion at a time convenient to both the layperson user and the healthcare professional. In some circumstances, both the healthcare professional and the layperson user would benefit from a suggested treatment protocol, the suggested treatment protocol optionally comprising a directive to consume certain types of hormone balancing seeds at certain times in the menstrual cycle as prompted by an application operating on the layperson user's computing device, which forms an aspect of the solution in an example. A single package comprising a specified quantity of diagnostic tests, the quantity of diagnostic tests optionally capable of evaluating a bodily fluid for the presence of hormones or analytes selected from the group of FSH, LH, PdG, an estrogen metabolite such as E3G and hCG each optionally corresponding to important indications and trigger points for the consumption of seeds at differing points in a single menstrual cycle, and/or a specified quantity of snack bars comprising pumpkin seeds and flax seeds and snack bars comprising sesame seeds and sunflower seeds, forms an aspect of the solution in an example. An application and/or a digital reader configured to evaluate the diagnostic tests and generate one or more unique messages related to the interpretations of the diagnostic tests, the unique messages optionally including prompts to consume a certain type of seeds at a certain time, forms an aspect of the solution in an example.

### Advantageous Effects of Invention

The examples of the invention as described herein provide a number of advantageous effects relevant to addressing undesirable conditions associated with the menstrual cycle, hormonal balances and the fertility of a patient. For example, the diagnostic tests comprising an aspect of the invention, when taken repeatedly on a daily basis over the course of a menstrual cycle in an example, provide an indication for the specific dates on which the phases of the menstrual cycle change. The results of such diagnostic tests can be automatically interpreted by an associated application, optionally operating on a smartphone capable of photographing the diagnostic tests to determine the color intensity indicated on such diagnostic tests. The application in an example is configured to detect the indications provided by either a single diagnostic test or a series of diagnostic tests and generate interpretations of the single diagnostic test or series of diagnostic tests as described herein. The results of such diagnostic tests and the generated interpretations may be delivered to a healthcare provider and made accessible to the healthcare provider via a communicatively connected storage medium for further evaluation of the patient, and during consultation with a patient during a telemedicine consultation, optionally triggered by one or more results of a diagnostic test as detected by the application or associated interpretations. The telemedicine consultation or the interpretations in an aspect are useful in generating a suggested treatment protocol. In one example, the suggested treatment protocol involves the consumption of one or more certain types of seeds based upon the detected result of the diagnostic test to address hormonal imbalances as described further herein.

### BRIEF DESCRIPTION OF DRAWINGS

FIG. 1 depicts a configuration of an embodiment of the diagnostic test featuring four testing zones and four corresponding result indication lines associated with aspects of the invention, and specifically the location of the result indication lines associated with an exemplary configuration of the diagnostic test.

FIG. 2 depicts an exploded view of an embodiment of the diagnostic test featuring four testing zones and four corresponding result indication lines associated with aspects of the invention, and specifically the location of the result indication lines associated with an exemplary configuration of the diagnostic test.

FIG. 3 depicts an exemplary diagnostic test key corresponding to an embodiment of the diagnostic test configured to evaluate specifically for the presence or absence of PdG at a threshold and the presence or absence of LH at a threshold.

FIG. 4 depicts an exemplary graphical user interface of the Healthcare Professional-Facing Application specifically configured to display a list of one or more patients and provide the capability to search for a patient.

FIG. 5 depicts an exemplary smartphone and an exemplary unique message.

FIG. 6 depicts an exemplary color intensity key.

FIG. 7 depicts an example of aspects of the system as used together to photographically capture a diagnostic test.

FIG. 8 depicts an example of aspects of the system as used together to photographically capture a diagnostic test in association with a stand featuring markings of a known distance apart intended to aid in the calculation of the dimensions of the diagnostic test.

FIG. 9 depicts an exemplary graphical user interface of the Patient-Facing Application specifically configured at least to display a list of one or more diagnostic test results, set a location, and schedule a telemedicine consultation.

FIG. 10 depicts various configurations of components of the system configured to either hold the diagnostic test for capture of a result and/or to display unique messages deriving from one or more interpretations of the indicated results.

FIG. 11 depicts an exemplary graphical user interface comprising a calendar.

FIG. 12 depicts an exemplary graphical user interface associated with the Scheduler associated with a specifically detected location of the user.

FIG. 13 depicts an exemplary graphical user interface associated with the seed consumption system, wherein the result of a diagnostic test is displayed in association with an interpretation displayed as a unique message.

FIG. 14 depicts an exemplary single consumable food item substantially in the form of a snack bar.

FIG. 15 depicts embodiments of the system comprising a plurality of diagnostic tests and a container.

FIG. 16. depicts an exemplary method of use of the system.

FIG. 17 depicts an exemplary method of use of a system associated with the scheduler.

FIG. 18 depicts an exemplary method of use of a system associated with the healthcare professional-facing application.

FIG. 19 depicts an exemplary method of use of a system associated with the patient-facing application.

FIG. 20 depicts an exemplary method of use of the telemedicine system.

FIG. 21 depicts an exemplary method of use of the telemedicine system incorporating testing for at least FSH and E3G.

FIG. 22 d

FIG. 23 depicts an exemplary method of determining results based on color intensity associated with various embodiments.

FIG. 24 depicts an exemplary method of generating an interpretation from one or more diagnostic tests associated with various embodiments.

FIG. 25 depicts an exemplary method of calibrating systems configured to interpret the indication or indications of one or more diagnostic tests.

FIG. 26 depicts an exemplary graphical user interface associated with the scheduler.

FIG. 27 depicts an exemplary method of progesterone supplementation.

### DESCRIPTION OF EMBODIMENTS

Menstrual Cycle Testing System

An embodiment of the invention comprises a menstrual cycle testing system comprising a Diagnostic Test, Camera, Patient-Facing Application, Healthcare-Facing Application, Storage, Graphical User Interface, Processor, Computing Device, Calendar, and Scheduler. Various other embodiments of the invention described herein incorporate and/or interact with the Menstrual Cycle Testing System. Various embodiments of the Menstrual Cycle Testing System comprise a Fertility Tracking System, Telemedicine System, Seed Consumption System, and/or Predicting Fertile Window System as described elsewhere herein.

Diagnostic Test

In the preferred embodiment, the diagnostic test consists of a lateral flow assay comprising at least one testing zone configured to detect for the presence or absence of pregnanediol glucuronide (PdG) in urine at a threshold selected from within the inclusive range of 1 μg/mL-10 μg/mL.

The technical specifications associated with the novel construction of the preferred embodiments of such diagnostic tests configured to evaluate for the presence or absence of at least pregnanediol glucuronide at a threshold are found within the following patent applications, with the benefit of priority claimed to each application: U.S. patent application Ser. No. 16/381,229 filed on Apr. 23, 2019; U.S. patent application Ser. No. 16/544,554 filed on Aug. 19, 2019; U.S. Patent Application 62/720,953 filed on Aug. 22, 2018; PCT Patent Application PCT/US18/68027 filed on Dec. 28, 2018; U.S. patent application Ser. No. 16/381,229 filed on Apr. 11, 2019; U.S. patent application Ser. No. 16/732,766 filed on Jan. 2, 2020; U.S. patent application Ser. No. 15/900,794 filed on Feb. 20, 2018, U.S. patent application Ser. No. 15/974,229 filed on May 2, 2018; U.S. Patent Application 62/720,953 filed on Aug. 22, 2019; and U.S. patent application Ser. No. 16/732,823 filed on Jan. 2, 2020, each of which are incorporated by reference herein with the benefit of priority claimed thereto. In the preferred embodiment, the diagnostic test configured to detect for the presence or absence of pregnanediol glucuronide comprises a lateral flow assay test comprising anti-pregnanediol glucuronide antibodies conjugated to visual label, optionally colloidal gold, and pregnanediol glucuronide conjugated to a globulin carrier protein, optionally bovine gamma globulin (BGG). In the preferred embodiment, the diagnostic test consisting of a lateral flow assay is configured to evaluate for the presence or absence of at least pregnanediol glucuronide at a threshold selected from within the range of 1 μg/mL-10 μg/mL incorporates specific reagent combinations uniquely enabling a strong enough interaction in the testing zone to allow for visual, naked eye inspection of the test results in accordance with the teachings in this disclosure and those incorporated by reference herein.

The present inventor has discovered that as pregnanediol glucuronide is a small hormone metabolite, in order to strongly bind to the surface of a membrane 193 of the diagnostic test 100, pregnanediol glucuronide requires a strong carrier protein. More specifically, the present inventor has determined, and it is a teaching of the preferred embodiment of the invention, that the diagnostic test 100 should be configured to facilitate the binding of 7 or more PdG molecules per carrier protein molecule to create enough reaction to enable a visual and/or optical indication for the presence or absence of PdG at a threshold in an applied fluid associated with the diagnostic test 100. In an example, such limitation is incorporated into the result indication line configured to provide a optical and/or visual result for the presence or absence of PdG at a threshold. The present inventor has recognized that by presenting more than 6 PdG molecules to each of the gold-conjugated anti-PdG antibodies, this results in the efficient binding and reactivity from the PdG-carrier protein and antibody, and thereby enables the proper functionality of the result indication line relevant to the presence or absence of PdG at a threshold in an applied fluid associated with the preferred embodiment of the diagnostic test 100.

In an example, the at least one additional testing zone, optionally consisting of the second testing zone, is placed onto the conjugate pad 190, corresponding to the second result indication line 108 placed onto the membrane 193, and configured to evaluate a fluid for the presence or absence of luteinizing hormone at a specified threshold selected from the range including 15 mlU/mL-50 mlU/mL. In an example of such configuration, at least a portion of the conjugate pad 190, or optionally the entirety of the conjugate pad 190, is sprayed at 2-5 ul/cm with a Potassium Phosphate buffer including 0.5% BSA, 0.1% surfactant, 5-15 OD anti-LH gold conjugate of concentration selected from the range inclusive of 6 ug/ml-8 ug/ml. In the example, a 1 mg/ml anti-LH antibody test is placed in a line across substantially the entire width of the membrane 193 to form a result indication line, optionally the second result indication line 108.

However, the present inventor has discovered that, for the preferred embodiment of the invention to function as intended, not only does the strong carrier protein need to bind the nitrocellulose membrane, but the strong carrier protein also needs to bind the pregnanediol glucuronide and present it to the anti-pregnanediol glucuronide antibody. Prior to the embodiments of the invention as disclosed herein, other attempts in the prior art have failed to include an optimal combination of a strong carrier protein able to bind the pregnanediol glucuronide and present it to the anti-pregnanediol glucuronide antibody. Such is the case for embodiments of the diagnostic test configured to detect for only the presence of the absence or presence of pregnanediol glucuronide at a threshold, and also embodiments of the diagnostic test configured to detect the presence of the absence or presence of pregnanediol glucuronide at a threshold in addition to detecting for the presence of other hormones and or analytes, such as luteinizing hormone (LH), in the same sample of fluid.

Another important component of the preferred embodiment of invention is the specifically chosen anti-pregnanediol glucuronide antibody. In order for the preferred embodiment of the invention to function as intended, the specifically chosen anti-pregnanediol glucuronide antibody must be monoclonal, due to the nature of the pregnanediol glucuronide antigen presentation on the BGG conjugate. In order for the embodiments of the invention to function as intended, the specifically chosen anti-pregnanediol glucuronide antibody must consist of one of the following isotypes: IgG1, IgG2a, IgG2b, or IgG2c. The present inventor has discovered that isotypes other than IgG1, IgG2a, IgG2b, or IgG2c, including but not limited to IgM, IgS, and IgE anti-pregnanediol glucuronide antibody isotypes, remain unable to effectively bind the colloidal gold (or other visual label) and likewise unable to produce a strong enough color signal on the reaction zone due to their size and structure and are therefore excluded from the preferred embodiment of the invention. Since the colloidal gold must bind the Ig region of the anti-pregnanediol glucuronide antibody, the present inventor has discovered that the IgG1, IgG2a, IgG2b, and IgG2c isotypes of the anti-pregnanediol glucuronide antibody sufficiently bind colloidal gold and are therefore incorporated into embodiments of the invention. As a result, the IgG1, IgG2a, IgG2b and IgG2c isotypes of the anti-pregnanediol glucuronide antibody therefore produce the strongest color. In the preferred embodiment of the invention, the IgG2b isotype is included in the invention, as the present inventor has recognized that the IgG2b isotype performs slightly better when producing color. Therefore, the preferred embodiment of the invention incorporates the IgG2b isotype of the anti-pregnanediol glucuronide antibody. Alternative embodiments incorporate the IgG2a, IgG2c or IgG1 isotypes of the anti-pregnanediol glucuronide antibody. In such embodiments, the conjugate striped on the membrane 193 in the test zone area is pregnanediol glucuronide-BGG and the anti-pregnanediol glucuronide antibody must be a monoclonal anti-pregnanediol glucuronide antibody of one of the following isotypes: IgG1, IgG2a, IgG2b, or IgG2c. Such is the case for embodiments of the diagnostic test configured to detect the presence of the absence or presence of pregnanediol glucuronide at a threshold alone, and also embodiments of the diagnostic test configured to detect the presence of the absence or presence of pregnanediol glucuronide at a threshold in addition to the presence of other hormones and/or analytes, such as luteinizing hormone (LH), in the same sample of fluid. Further details and teachings associated with the construction of such a diagnostic test configured to detect for the presence or absence of at least pregnanediol glucuronide are disclosed in at least U.S. Patent Application 62/720,953 filed on Aug. 22, 2018 which is incorporated by reference in its entirety with priority claimed thereto.

In the preferred embodiment, a fluid sample is applied to the lateral flow assay as described in the above referred-to patent applications. By lateral flow, the sample is allowed to permeate through the strip material into or through the one or more testing zones, whereby which the conjugate pad 190 of the diagnostic test 100 is coated with specific binding partners, optionally as described elsewhere herein. In an example, pregnanediol glucuronide from the fluid sample becomes bound within the first testing zone and the extent to which analyte becomes bound can be determined by labeled secondary reagents such as colored latex beads or colloidal gold. The color intensity in each result indication line in an example is correlated with the concentration of the analyte in the fluid sample, optionally as indicated on the Diagnostic Test Key 200. In the preferred embodiment, the diagnostic test 100 is configured to evaluate for the presence or absence of at least pregnanediol glucuronide at a threshold selected from within the range of 1 μg/mL-10 μg/mL is single-use and disposable after results are indicated. In an embodiment, the diagnostic test 100 comprises with a result indication line configured to generate a visual or optical signal for the presence or absence of pregnanediol glucuronide at a threshold of 5 μg/mL.

In an embodiment, the diagnostic test consists of a single lateral flow assay test configured to simultaneously or near-simultaneously detect for the presence or absence of pregnanediol glucuronide at a threshold in addition to detecting for the presence of one additional hormone or hormonal analyte or a plurality of additional hormones or hormonal analytes selected from the group consisting of luteinizing hormone, estrogen, estriol glucuronide, estradiol, progesterone and human chorionic gonadotropin, each optionally at a (distinct) threshold, in the same fluid sample, with each indication provided in association with a separate result indication line. One example of such a test is further described in U.S. patent application Ser. No. 16/381,229, filed on Apr. 11, 2019, which is incorporated by reference herein in its entirety with claim of priority thereto. For instance, the present inventors have recognized the benefit of a configuration of the diagnostic test 100 embodied as a lateral flow assay configured to simultaneously analyze urine beyond mere analysis for the presence or absence of pregnanediol glucuronide at a threshold, by further analyzing up to six additional analytes and/or hormones each in a separate testing zone located distal from the fluid application zone 106 on the same lateral flow assay. The present inventor has noted that due to the peculiarities associated with detecting PdG in urine, and the potential for interference from other testing zones, it is important in an embodiment to configure the lateral flow assay to ensure that the applied fluid travels through the first testing zone and first result indication line 107 configured to detect for the presence or absence of PdG at a threshold prior to traveling through any other result indication line on the same lateral flow assay. In an embodiment, the diagnostic test 100 incorporates a first testing zone located within the conjugate pad 190 and a first result indication line 107 and a control line 105, and no other testing zones or result indication lines. In an embodiment, the diagnostic test 100 incorporates a first testing zone and second testing zone located within the conjugate pad 190 and a first result indication line 107 and a second result indication line 108 and a control line 105, and no other testing zones or result indication lines.

In an embodiment, the diagnostic test 100 consists of a lateral flow assay comprising a conjugate pad 190 comprising at least a first testing zone configured to evaluate urine for the presence or absence of pregnanediol glucuronide at a specific threshold, and additionally at least a second testing zone and optionally a third testing zone and/or fourth testing zone located within the conjugate pad 190. In various embodiments, the first testing zone, the second testing zone, the third testing zone and the fourth testing zone each occupy distinct areas on the conjugate pad 190 along the entire width of the conjugate pad 190, configured such that applied fluid must come into contact with each applied testing zone as it travels through the conjugate pad 190 to come into contact with the membrane 193 during lateral flow of the applied fluid. In an alternative embodiment, at least two of the testing zones are applied together, in which case the various conjugates may either be mixed prior to application or applied sequentially in an overlapping fashion onto the same area of the conjugate pad 190. The present inventor has recognized various advantages and disadvantages to either approach. The present inventor has noted a higher precision and a lower risk of cross-contamination with regard to the indicated results in the example where the testing zones each occupy distinct areas on the conjugate pad 190. However, the present inventor has also noted that it is easier to manufacture a diagnostic test 100 in the example where at least two of the testing zones are applied together. Both approaches are incorporated as teachings relevant to embodiments of the invention. Regardless of the method of application, it is a teaching of an embodiment of the invention for the diagnostic test 100 to optionally incorporate one or more additional testing zones located within the conjugate pad 190, optionally a second testing zone, a third testing zone, and a fourth testing zone, beyond the first testing zone configured to analyze urine for the presence or absence of pregnanediol glucuronide, and further to incorporate a result indication line on the membrane 193 corresponding to each testing zone configured to provide an indication for the presence, the indication for the presence optionally consisting of a visual signal corresponding to the presence or absence at a threshold, of an item selected from the group consisting of LH, HCG, FSH, Testosterone and/or Estrogen or an estrogen metabolite such as E3G.

In an embodiment of the diagnostic test 100, the diagnostic test 100 consists of a lateral flow assay comprising a sample pad 191 configured to receive an applied fluid and transfer the applied fluid to the conjugate pad 190, and following the transit of the applied fluid to the conjugate pad 190 (and its at least one testing zone) to a membrane 193 comprising at least one result indication line, optionally the first result indication line 107, configured to provide indication for the presence or absence of pregnanediol glucuronide at a specified threshold selected from the range including 1 μg/mL-10 μg/mL in the applied fluid. In an embodiment, during the manufacture of the diagnostic test 100, its conjugate pad 190 is at least sprayed at 2-6 ul/cm with a Potassium Phosphate buffer including 0.5% BSA, 0.1% surfactant, 5-15 OD anti-PdG gold (at a concentration selected from the range of Zug/ml-8 ug/ml) to forma testing zone, optionally the first testing zone. In such embodiment, the membrane 193 is separately and additionally applied with a conjugate of PdG-BGG in an amount of 0.5 mg/ml, placed in a line across substantially the entire width of the membrane 193, which provides a configuration able to display an indication (optionally an optical signal) in the form of a result indication line for the presence or absence of PdG at threshold amount of 5 μg/mL in an applied fluid. In an alternative embodiment, the membrane 193 is applied with a conjugate of PdG conjugated to a mixture of complement factor H (in an embodiment, comprising 15% of the mixture), BSA (in an embodiment, comprising 20% of the mixture), BGG (in an embodiment, comprising 50% of the mixture), beta-2-glycoprotein (in an embodiment, comprising 10% of the mixture), compliment favor B (in an embodiment, comprising 5% of the mixture), placed in a line across substantially the entire width of the membrane 193, thereby configured to display an indication (optionally an optical signal) in the form of a result indication line, optionally the first result indication line 107, of the presence or absence of PdG at an amount selected from the range inclusive of 1 μg/mL-10 μg/mL based upon the relative concentration of such elements in an applied sample, in the form of and to create at least one result indication line. In various examples, the membrane 193 is further applied with a 1.2 mg/ml goat anti-mouse antibody placed in a line across substantially the entire width of the membrane distal from the fluid application point relative to any applied result indication lines in the form of and to create a control line 105. In an example, each result indication line and control line is simultaneously (or as necessary, sequentially) dispensed onto the membrane using a BioDot XYZ3210 dispenser, and allowed to dry following application.

In various embodiments, to facilitate the testing of additional hormones and/or hormone metabolites, the conjugate pad 190 is additionally sprayed with, optionally in distinct areas encompassing the entire width of the conjugate pad 190 or optionally all together encompassing substantially the entire area of the surface of the conjugate pad 190, one or more additional buffers each comprising a testing zone (alternatively each referred to as a “receiving zone”). Corresponding to each of the one or more additional testing, a separate result indication line is placed onto the membrane 193 in substantially the same manner but with a unique conjugate configured to create a visual indication line corresponding to the hormone or hormone analyte evaluated in association with the pertinent testing zone.

In an example, the at least one additional testing zone, optionally consisting of the second testing zone, is placed onto the conjugate pad 190 and configured to evaluate a fluid for the presence or absence of luteinizing hormone at a specified threshold selected from the range including 15 mlU/mL-50 mlU/mL. In an example of such configuration, at least a portion of the conjugate pad 190, or optionally the entirety of the conjugate pad 190, is sprayed at 2-5 ul/cm with a Potassium Phosphate buffer including 0.5% BSA, 0.1% surfactant, 5-15 OD anti-LH gold conjugate of concentration selected from the range inclusive of 6 ug/ml-8 ug/ml. In the example, an anti-LH antibody at a concentration of 1 mg/ml is sprayed in a line across substantially the entire width of the membrane 193 to form a result indication line, optionally the second result indication line 108.

During an example of the method of assembly, a step of applying a plurality of testing zones and the control line to the membrane 193 simultaneously is performed. During such step, the PdG test line forming the first result indication line 107, the LH test line forming the second result indication line 108, and the control line 105 are simultaneously dispensed onto the membrane 193 component of the lateral flow assay using a dispenser, optionally a BioDot XYZ3210 and allowed to dry. Further during such method of assembly, a step of creating a plurality of conjugates is performed. During such exemplary step, a first conjugate is created of Potassium Phosphate buffer including 0.5% BSA or 0.5% BGG, 0.1% surfactant, 5-15 OD anti-PDG gold (conc. 2-8 ug/ml), and a second conjugate is created of Potassium Phosphate buffer including 0.5% BSA, 0.1% surfactant, 5-15 OD anti-LH gold (conc. 6-8 ug/ml). Further during such method of assembly, a step of spraying the plurality of conjugates onto the conjugate pad 190 of the lateral flow assay is performed, wherein the first conjugate is sprayed at 2-6 ul/cm onto at least the portion of the conjugate pad 190 corresponding to the first testing zone and the second conjugate is sprayed at 2-5 ul/cm onto at least the portion of the conjugate pad 190 corresponding to the second testing zone. In an embodiment the portions of the conjugate pad first testing zone and second testing zone (and optionally other testing zones) partially or fully overlap, and optionally are mixed together prior to application such that all testing zones are pre-mixed and applied in a single spray to a single area onto the conjugate pad 190. A further step of the method comprises forming a mastercard. During such step, the membrane 193 and conjugate pad 190 components are applied, further comprising a sample pad 191, optionally blocked with 0.1 M Borate buffer including 1% PVP-10 and 1% detergent and a wicking pad optionally consisting of a 901 Ahlstrom wicking pad, are attached to a backing card (the backing card comprising one of those readily available and known in the art). During such step, the membrane 193 (also optionally referred to as the “testing zone”), conjugate pad 190 (also optionally referred to as the “receiving zone”), sample pad 191 (also optionally comprising the fluid application zone 106), wicking pad (also optionally referred to as the “adsorbent pad,” which collects the applied fluid following lateral flow to mitigate leakage) and backing card 194 are attached together by a clamshell laminator, optionally the BioDot LM5000 Clamshell Laminator. The method further comprises cutting the mastercard into strips. During such step, the mastercard is cut into strips optionally with a guillotine cutter, optionally the BioDot CM4000 Guillotine Cutter, each strip then ready for subsequent placement into a sealed aluminum pouch with a desiccant for preservation.

In an embodiment, the diagnostic test consisting of a lateral flow assay is configured to simultaneously indicate a positive or negative result for the presence or absence of pregnanediol glucuronide at a threshold in a sample of urine applied to the lateral flow assay. In one example of the diagnostic test, in addition to indicating a positive or negative result for the presence or absence of pregnanediol glucuronide at a threshold, in a second testing zone, a positive or negative result for the presence of at least one additional analyte and/or hormone, and, optionally, indicating in an third testing zone, a positive or negative result for the presence of at least one additional analyte and/or hormone, each contained within a single diagnostic test consisting of a lateral flow assay. In an embodiment of the invention, the at least one additional analyte and/or hormone to be tested within the second testing zone and optionally the at least one additional analyte and/or hormone to be tested within the third testing zone is selected from the following group: (1), estradiol (E2) with a threshold set at a concentration chosen from the range inclusive of 25-250 pg/ml in a competitive assay format; (2), follicle stimulating hormone (FSH) with a threshold set at a concentration chosen from the range inclusive of 3-20 mlU/ml in a sandwich assay format; (3), luteinizing hormone (LH) with a threshold set at a concentration chosen from the range inclusive of 0-25 mlU/ml in a sandwich assay format; (4), progesterone (P4) with a threshold set at a concentration chosen from the range inclusive of 0-40 ng/ml in a competitive or sandwich assay format; (5) human chorionic gonadotropin, (hCG) with a threshold set at a concentration chosen from the range inclusive of 0-10,000 mlU/ml; and Testosterone, with a threshold set at a concentration chosen from the range inclusive of 0 to 50 μg, in a sandwich assay format. In an embodiment, the second testing zone is configured to detect for the presence of a hormone or analyte differing from the hormone or analyte detected by the third testing zone. In an embodiment, the diagnostic test consists of a lateral flow assay comprising at least one testing zone configured to detect for the presence or absence of pregnanediol glucuronide in urine at a threshold selected from within the range of 1 μg/mL-10 μg/mL further comprising one or more additional testing zones each configured to detect for the presence or absence of a hormone or hormonal analyte selected from the group consisting of: estradiol or estriol glucuronide (E3G) at a threshold selected from the range inclusive of 25 ng/mL-250 ng/mL, follicle stimulating hormone at a threshold selected from the range inclusive of 3 mIU/ml-20 mIU/ml, luteinizing hormone at a threshold selected from a range inclusive of 15 mIU/ml-50 mIU/ml, human chorionic gonadotropin at a threshold selected from the range inclusive of 1 mIU/ml-10,000 mIU/ml, and progesterone at a threshold selected from the range inclusive of 1 ng/mL-40 ng/mL.

In various examples of the diagnostic test 100, the one or more testing zone(s) corresponding to the hormone or hormonal analyte selected from the group consisting of LH, FSH, hCG and/or an estrogen metabolite such as E3G, are applied in a similar manner to the testing zone configured to facilitate evaluation of an applied fluid for PdG as described herein. In an exemplary method of assembly comprising the step of creating a plurality of conjugates, one or more additional conjugates are created. For example, a FSH conjugate comprising a Potassium Phosphate buffer including 0.5% BSA, 0.1% surfactant, 5-15 OD anti-FSH gold (conc. 3-8 ug/ml) is created, in which case the step of spraying the conjugate onto the conjugate pad 190 further comprises spraying the FSH conjugate at 2 ul/cm-4 ul/cm onto at least the portion of the conjugate pad 190 corresponding to a testing zone, optionally the third testing zone. In association with the testing zone configured to evaluate for FSH, the applying a plurality of testing zones and the control line to the membrane 193 simultaneously step further comprises applying in the testing zone configured to evaluate for FSH the 1 mg/ml anti-FSH antibody test line, optionally comprising the third result indication line 109. Also for example during the creating a plurality of conjugates step, a hCG conjugate comprising Potassium Phosphate buffer including 0.5% BSA, 0.1% surfactant, 5-15 OD anti-HCG gold (conc. 2-9 ug/ml), in which case the step of spraying the conjugate onto the conjugate pad 190 further comprises spraying the hCG conjugate at 2-6 ul/cm onto at least the portion of the conjugate pad 190 corresponding to a testing zone, optionally the fourth testing zone. In association with the testing zone configured to evaluate for hCG, the applying a plurality of testing zones and the control line to the membrane 193 simultaneously step further comprises applying in the testing zone configured to evaluate for hCG by striping 1.5 mg/ml anti-HCG antibody in the test line area, optionally comprising the fourth result indication line 110.

In an embodiment of the invention, the diagnostic test is configured as a lateral flow assay comprising a first testing zone configured to simultaneously indicate a positive or negative result for the presence or absence of pregnanediol glucuronide at a threshold selected from the range inclusive of 1 μg/mL-10 μg/mL in a sample of urine and a second testing zone configured to evaluate urine for the presence or absence of luteinizing hormone at a specified threshold selected from the range including 15 mlU/mL-50 mlU/mL, with the diagnostic zones located in a specific order at specific locations on the diagnostic test corresponding to match instructions provided on a printed material located within a system further comprising a plurality of diagnostic tests, or otherwise a digitized or programmed Diagnostic Test Key 200 configured as further described elsewhere herein optionally also included within the system. In one embodiment, each individual diagnostic test is configured to evaluate for the presence or absence of both progesterone glucuronide at a threshold and luteinizing hormone at a threshold in a single lateral flow assay, packaged together in quantity. In an embodiment, a plural quantity of diagnostic tests is a specific teaching of the system, the quantity correlating to the testing needs associated with one menstrual cycle as further described elsewhere herein. In one exemplary fertility testing system, the fertility testing system comprises a quantity of diagnostic tests each configured to evaluate for the presence or absence of both progesterone glucuronide at a threshold and luteinizing hormone at a threshold in a single lateral flow assay in a quantity selected from the range inclusive of 10 through 35 diagnostic tests. The present inventor has recognized that such a range of quantities of diagnostic tests configured to evaluate for the presence or absence of both progesterone glucuronide and luteinizing hormone in a single lateral flow assay adequately would allow for daily or twice daily testing over the relevant timeframe to collect enough results for the presence or absence of progesterone glucuronide at a threshold and luteinizing hormone at a threshold, and optionally the presence or absence of other hormones and/or analytes each at a threshold, to capture useful information relevant to fertility tracking over a single menstrual cycle. It is therefore a step of the method of use of the system to engage in determining a result for the presence or absence of at least pregnanediol glucuronide in the woman's urine via an indication generated by a lateral flow assay test comprising at least one testing zone configured to detect for the presence or absence of pregnanediol glucuronide in urine at a threshold selected from within the range of 1 μg/mL-10 μg/mL 2001.

In an embodiment, the diagnostic test is intended for use in association with a digital reader 670 configured to evaluate at least the second testing zone and optionally the third testing zone, the second testing zone and third testing zone each configured to evaluate for the presence or absence of a distinct hormone or analyte each at a threshold, in association with the methods described herein. In an embodiment, the diagnostic test incorporates, in addition to a first testing zone configured for the presence or absence of pregnanediol glucuronide at a threshold as described herein, a second testing zone configured to evaluate for the presence or absence of second hormone or analyte (optionally LH) at a threshold, and a third testing zone configured to evaluate for the presence or absence of third hormone or analyte (optionally E3G or FSH) at a threshold, and a fourth testing zone configured to similarly detect for the presence of a fourth hormone or analyte differing from the hormone(s) or analyte(s) detected by the other (first, second and third) testing zones (optionally FSH or hCG) within the diagnostic test. The methods of evaluation and further configurations optionally applied to the diagnostic test, optionally configured as a test strip, associated with the testing of analytes and/or hormones beyond and in addition to pregnanediol glucuronide are further described in at least U.S. patent application Ser. No. 15/974,229, filed on May 25, 2018, which is hereby incorporated by reference in its entirety.

Importantly, the present inventor has recognized that LH and hCG commonly exhibit cross-reactivity, specifically due to the fact that hCG can bind to LH antibodies. Therefore, having different colors corresponding to the presence or absence of different hormones and/or analytes each at a distinct threshold provides a benefit by allowing an observer to determine whether cross-reactivity has taken place with or without the assistance of a digital reader 670. For example, if an area designated to test for the presence of LH displayed the coloration of the label for hCG, such a result indicates that cross-reactivity has been demonstrated, and it is a teaching of an embodiment that an indicated cross-reactivity would render the diagnostic test result invalid. Alternatively, the present inventor has noted that due to the similarities in structure between estrogen analytes and progesterone analytes (in at least one example, said progesterone analytes consisting of PdG), cross reactivity may take place between those two hormone metabolites specifically. Thus, the present inventor has discovered and it is a teaching of an embodiment to incorporate a plurality of testing zones each configured to evaluate a different hormone and/or hormone metabolite with the indication associated with different colors. In an example, in part to mitigate the effects of the cross-reactivity, it is a teaching of the invention to order the testing zones in such a manner that cross-reactivity is minimized. In one example, the first testing zone is configured to evaluate a fluid for the presence or absence of PdG at a threshold, the second testing zone is configured to evaluate a fluid for the presence or absence of LH at a threshold, the third testing zone is configured to evaluate for the presence of FSH, and the fourth testing zone is configured to evaluate for the presence or absence of hCG at a threshold, each testing zone located within the conjugate pad 190 and corresponding to a specific result indication line located within the membrane 193. Such labelling of the different tested hormone and hormone metabolites is accomplished in an embodiment by binding to colloidal gold and/or one or more differently colored latex beads, each testing zone within the exemplary diagnostic test 100 featuring a differently colored label, optionally as further described elsewhere herein.

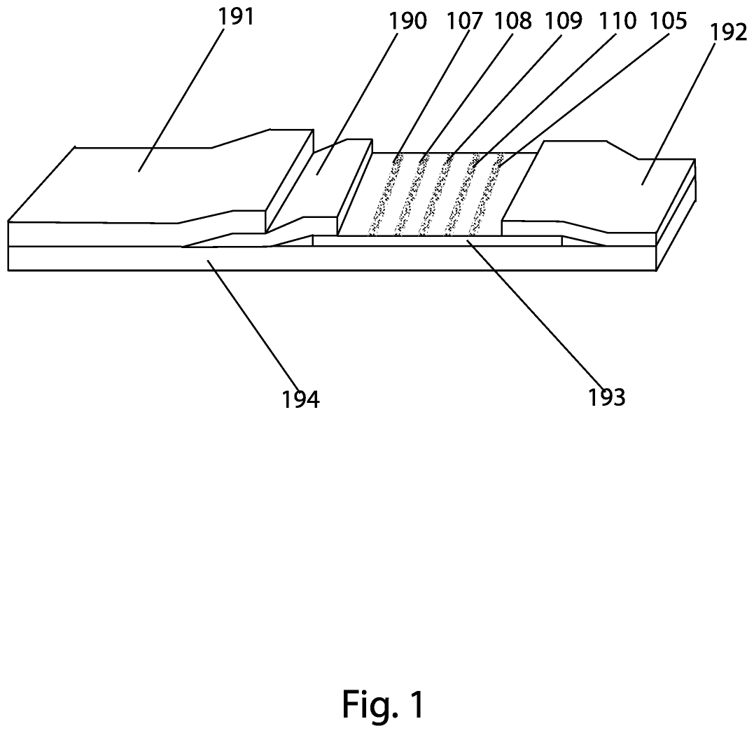

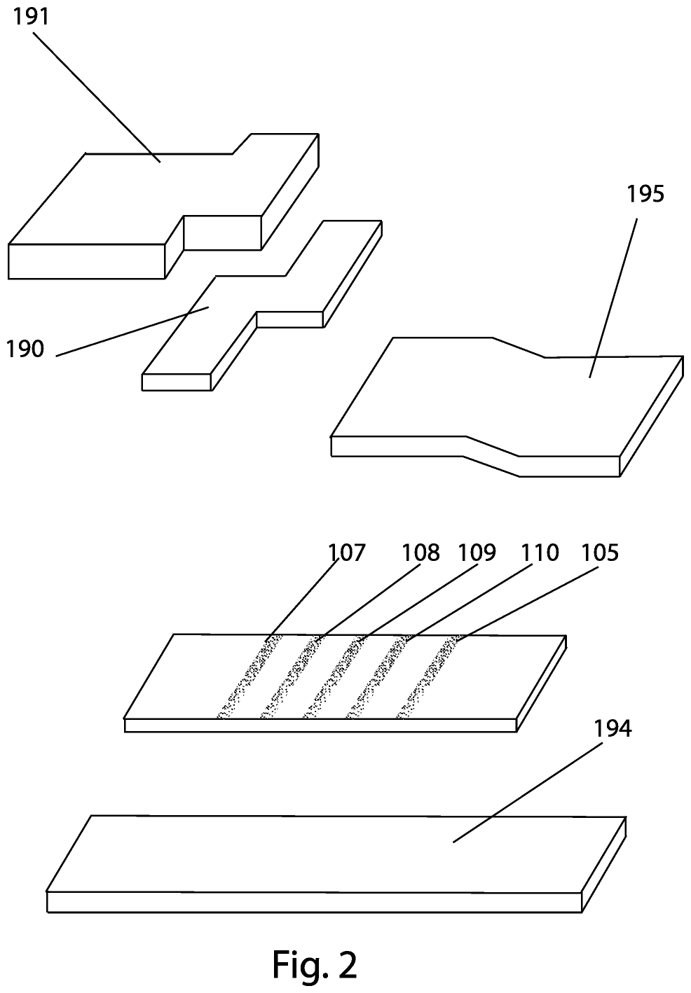

In an embodiment, the diagnostic test 100 consisting of a lateral flow assay is configured to incorporate a plurality of result indication lines, each result indication line at a specified distance and/or in a specified sequence from one end of the lateral flow assay, and each result indication line corresponding to an optically and/or visually perceptible label to indicate the presence of a distinct hormone or hormonal analyte, or optionally the presence or absence of one distinct hormone or hormonal analyte at a threshold, in a fluid sample placed into contact with the lateral flow assay. It is a teaching of an embodiment of the invention to configure the diagnostic test 100 to incorporate precise measurements and/or a specified sequence of hormones and/or analytes to identify the distance from at least one end of the lateral flow assay to each result indication line and/or the specified sequence from one end 111 of the lateral flow assay to aid in the interpretation of any result indicated by each result indication line of the lateral flow assay. An example of such a sequence with such measurements is depicted by FIG. 1 and FIG. 2, where in an example the first testing zone and the first result indication line 107 is configured to detect and indicate for the presence or absence of PdG at a threshold, the second testing zone and the second result indication line 108 is configured to detect and indicate for the presence or absence of LH at a threshold, the third testing zone and the third result indication line 109 is configured to detect and indicate for the presence of E3G, and the fourth testing zone and the fourth result indication line 110 is configured to detect and indicate for the presence of FSH. The sequence of the testing zones or distance of each testing zone from at least one of the ends 111 of the lateral flow assay, which in varying embodiments may vary according to the configuration, quantity or type of hormones and/or analytes detected, is then used to identify the hormone or analyte tested within each testing zone, with a indication provided by the corresponding result indication line, optionally in association with the use of a Diagnostic Test Key 200 as described elsewhere herein, and optionally in conjunction with a digital reader 670 as described elsewhere herein and within the disclosures incorporated by reference herein. In an exemplary configuration, the first result indication line 107 is located 29 mm from the end of the diagnostic test 111, the second result indication line 108 is located 32 mm from the end of the diagnostic test 111, the third result indication line 109 is located 35 mm from the end of the diagnostic test 111, and the fourth result indication line 110 is located 38 mm from the end of the diagnostic test 111 in an embodiment of the invention. In an example, the first result indication line 107 provides an indication for the presence or absence of pregnanediol glucuronide at a threshold in a fluid sample applied to the diagnostic test 100 at the fluid application zone 106, the second result indication line 108 provides a result for the presence or absence of luteinizing hormone in a fluid sample applied to the diagnostic test 100 at the fluid application zone 106, the third result indication line 109 provides a color intensity result pre-correlated to a concentration of E3G in a fluid sample applied to the diagnostic test 100 at the fluid application zone 106, and the fourth result indication line 110 provides a color intensity result pre-correlated to a concentration of FSH in a fluid sample applied to the diagnostic test 100 at the fluid application zone 106. In one example, the digital reader 670 and/or the Patient-Facing Application, optionally in association with other elements of the system, is programmed to associate one indication or a plurality of indications detectable in association with result indication lines on a lateral flow assay each located at the distances as specified above, or at alternative distances in accordance with the specific configuration of the lateral flow assay, from at least one end 111 of the lateral flow assay. Each specific indication present at a distance, optionally in association with a color intensity as described elsewhere herein, is pre-associated with an interpretation for the presence or absence of specific hormones and or analytes, each optionally at a threshold or in association with a pre-determined concentration, in accordance with the teachings elsewhere herein. It is a teaching of an embodiment that the manufacturing processes associated with such lateral flow assays are so configured to reproduce lateral flow assays in a standardized manner such that the digital reader 670 and/or the Patient-Facing Application, optionally in association with other elements of the system, may be preprogrammed with the one distance or plurality of distances from one end of the lateral flow assay, and the association of the one distance or plurality of distances from one end 111 of the lateral flow assay each with a distinct hormone or analyte. Additionally, the digital reader 670 and/or the Patient-Facing Application in an embodiment is additionally pre-programmed with the color intensity, with is optionally determined in advance by spiking a sample of male urine with the specific threshold of hormone or analyte and detecting the color intensity displayed in a testing zone following the application of the sample to the lateral flow assay to establish the color intensity associated with the threshold to indicate the presence or absence of such hormone or analyte for subsequent use in association with configuring the system.

It is a teaching of an embodiment of the invention to utilize the color intensity correlation to the threshold to facilitate the collection and digitization of a result of the diagnostic test 100 optionally consisting of a lateral flow assay in accordance with the teachings elsewhere herein, and to pre-program the Patient-Facing Application and/or digital reader 670, optionally in association with the other elements of the system, by use of such color intensity or color intensities of any subset of the group consisting of the first result indication line 107, the second result indication line 108, the third result indication line 109, and the fourth result indication line 110, each of the subset associated with a specific hormone and/or analyte at a threshold, and optionally each associated with a specific distance from one end 111 of the lateral flow assay, to preconfigure and enable functioning of the system in accordance with coding and computer programming mechanisms as well understood by those skilled in the art. In an example, the Diagnostic Test Key 200 is configured to incorporate such color intensity or color intensities to a threshold or thresholds of different hormones and/or analytes, optionally at specified locations on the diagnostic test 100 consisting of a lateral flow assay. In an embodiment, the Diagnostic Test Key 200 is preconfigured within the digital reader 670, Patient-Facing Application and/or smartphone 600, optionally in association with other elements of the system, via coding and computer programming mechanisms as well understood by those skilled in the art. In one example of the above, a Smartphone featuring a Camera is utilized to photograph and identify the shape of the diagnostic test 100 consisting of a lateral flow assay and calculate the distance from one end 111 with preprogrammed dimensions of the lateral flow assay, and optionally via the use of the Pythagorean Theorem, to determine the dimensions of the lateral flow assay and location of each of any subset of the group consisting of the first result indication line 107, the second result indication line 108, the third result indication line 109 and the fourth result indication line 110 on the diagnostic test 100 to accomplish the recognition of each indications, optionally by calculating the color intensity in each testing zone and comparing it to the pre-programmed color intensity of the threshold, or optionally by detecting the HEX or RGB color number displayed on each result indication line and comparing it to a pre-programmed HEX color number, RGB color number or other color identifier associated with a specific quantity of hormone or hormone analyte optionally in comparison to the HEX color number, RGB color number or other color identifier associated with one or more colors displayed on the color intensity key and/or the Diagnostic Test Key 200 and associated with a quantity of the relevant hormone or hormone analyte, on the lateral flow assay. In exemplary embodiments, the sequence of the testing zones is detected and interpreted with the assistance of one or more digital readers 670, in association with methods as well understood by those in the art, such as those described in PCT Patent Application PCT/CN2017/085010 filed on May 19, 2017 and corresponding U.S. patent application Ser. No. 16/302,085 filed on May 29, 2019, and PCT Patent Application PCT/US2018/038173 filed on Jun. 20, 2019 claiming priority to U.S. Patent Application 62/688,970, each of which is incorporated by reference.

In one exemplary embodiment, as depicted in FIGS. 1b and 2b, the second result indication line 108 is configured to provide an indication for the presence or absence of luteinizing hormone at a threshold, the first result indication line 107 is configured to provide an indication for the presence or absence of progesterone glucuronide and the control line 105 is configured to provide a visual indication upon the application of any fluid, to ensure that the fluid has passed through each of the testing zones and each of the result indication lines present within the diagnostic test 100 from the fluid application zone 106. In various embodiments, the control line 105 is the most distal from the fluid application zone 106, and must pass through the one or more testing zones and the one or more result indication lines to provide a visual indication that the lateral flow assay has been performed correctly. Each analyte and/or hormone tested in each testing zone of a diagnostic test 100 as indicated by a corresponding result indication line, and/or the control line 105, optionally corresponds to a different label to produce a distinct color (such as colloidal gold and or latex beads).

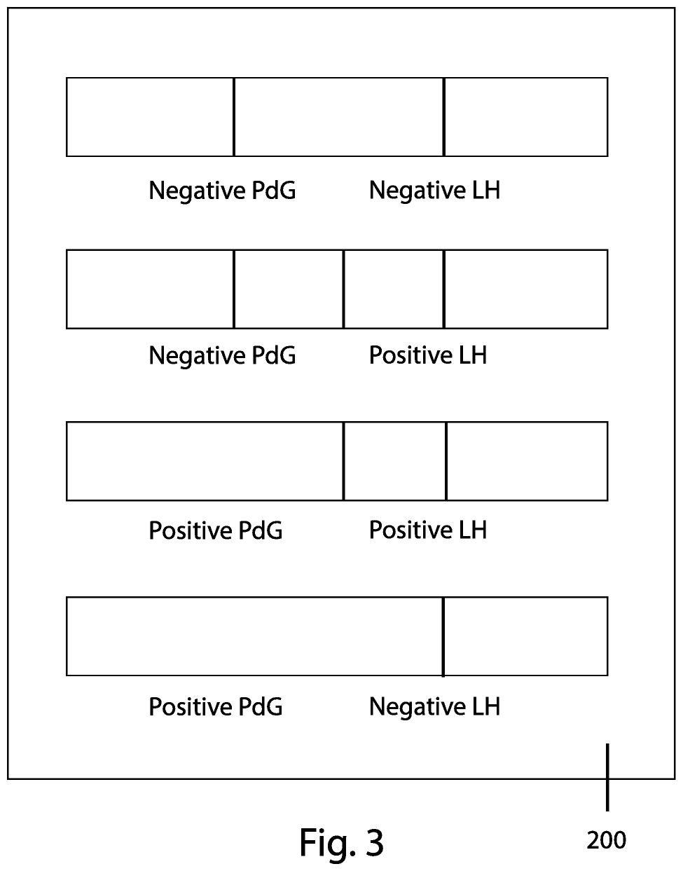

As illustrated by FIG. 1b in an example, the first result indication line 107 appears on the lateral flow assay at a distance selected from within the range of 25-45 mm and the second result indication line 108 appears at a specified and differentiated distance on the lateral flow assay from a distance range selected from the range including 28-50 mm from the end 111 at which a fluid is applied. In an embodiment, the third result indication line 109 appears on the lateral flow assay at a distance selected from within the range of 35-55 mm and the fourth result indication line 110 appears at a specified and differentiated distance on the lateral flow assay from a distance range selected from the range including 38-58 mm from the end 111 at which a fluid is applied. In various embodiments, the control line 105 is located distal from the end 111 at which a fluid is applied at a distance selected from the range of 2-10 mm from the edge of the testing zone located most distal from the end 111 at which a fluid is applied. In an embodiment, the first result indication line 107 appears on the lateral flow assay at a distance of 29 mm from the end 111 at which a fluid is applied, the second result indication line 108 appears on the lateral flow assay from a distance range of 32 mm from the end 111 at which a fluid is applied. In an embodiment, the specific sequence of the each result indication line (or the locations thereof, for example, in the absence of an indicated line where the absence of an indicated line at the location of a result indication line signifies a result) on the diagnostic test 100 and the control line 105 is associated with a specific sequence of hormones and/or analytes tested by the diagnostic test 100 and correlates to information provided in association with a Diagnostic Test Key 200 which optionally is included within system embodiments of the invention, or pre-programmed on a digital reader 670 or a Patient-Facing Application for use in association with associating the each result indication line with a specific hormone or hormone analyte. In an embodiment, the sequence of each result indication line depicted in association with a Diagnostic Test Key 200 is utilized as an alternative to distinct color labeling of each analyte and/or hormone tested to provide an representation of which distinct analyte and/or hormone is indicated on each result indication line of a diagnostic test 100. In an embodiment, the presence or absence of result indication lines in a pre-defined sequence is programmed and/or coded in association with a Patient-Facing Application which may be utilized to detect the presence or absence of each hormone as a result of the specific indications of a diagnostic test 100. The present inventor has recognized the importance of external mechanisms such as the Patient Facing Application and/or Diagnostic Test Key 200 in association with the utilization of the preferred embodiment of the diagnostic test 100, as some hormones or analytes such as PdG are associated with the absence of a visual indication line to indicate a positive result, whereas distinct hormones or analytes such as LH are associated with the presence of a visual indication line to indicate a positive result. Further, especially due to the possibility that the presence of one line may indicate a positive result and the presence of a separate and distinct line on the same strip may indicate a negative result, for instance as indicated on the printed Diagnostic Test Key 200 embodiment illustrated by FIG. 3, an external mechanism such as a printed Diagnostic Test Key 200, a Patient Facing App, or a Diagnostic Test Key coded into a Patient-Facing Application is transformative in facilitating layperson understanding of the visual results indicated on a diagnostic test 100. It will be appreciated by those skilled in the art that a variety of diagnostic tests for uses in association with a variety of contexts may be collected, read and interpreted by the Patient-Facing Application, for example either by color labeling or by the sequence of the hormones and/or analytes being tested on the diagnostic test 100.

In various embodiments of the invention, diagnostic test 100 consisting of a lateral flow assay further comprises a visual label configured to display or not display a specific color based indicating the presence or absence of a hormone or hormonal analyte within a fluid sample placed into contact with the lateral flow assay. In an example, the presence or absence of a color at a specified intensity provides an indication of the presence or absence of a hormone or hormonal analyte within a fluid sample placed into contact with the lateral flow assay. In an example of the diagnostic test, the labels (such as colloidal gold) are varied, with a separate and distinct label configured to attach to a separate and distinct hormone or analyte. In such example, the present inventor has recognized the advantage that the diagnostic test 100 is configured to provide a different color for each distinct hormone and analyte indicating either the presence or absence of each hormone analyte at a threshold following application of urine to the diagnostic test. In an embodiment, the diagnostic test 100 is configured as a lateral flow assay comprising a conjugate pad 190 (the conjugate pad also optionally referred to as the “receiving zone”) comprising anti-PdG antibody-collodial gold conjugate placed to form a testing zone, and at least one other conjugate placed within another testing zone. In an embodiment, the at least one other conjugate comprises anti-LH antibody-conjugated with a different label, optionally differently colored latex beads. In various embodiments, the configurations of the diagnostic test 100 featuring a different color representing an indication of the presence or absence of a distinct hormone or analyte are as described in U.S. patent application Ser. No. 16/381,229 filed Apr. 11, 2019, and PCT Application No. PCT/US18/68027, filed Dec. 28, 2018, each of which are incorporated by reference with priority claimed thereto.

In accordance with such teachings and the components of the system as described elsewhere herein, the method of use of the system comprises the step of determining a result from a lateral flow assay test configured to detect for at least one additional hormone or hormonal analyte (other than PdG) from the group consisting of: the presence or absence of luteinizing hormone at a threshold at a threshold, the presence or absence of and human chorionic gonadotropin at a threshold, the presence of E3G in a concentration correlating to a color intensity, and the presence of FSH in a concentration correlating to a color intensity 2009.

The diagnostic test 100 configured as described herein has numerous advantageous not previously known in the art. For example, the configurations as described herein are easily performed and recorded—optionally in association with a Patient-Facing Application, the Seed Consumption System, and/or a mechanism to trigger the prescription and delivery of progesterone supplementation to a patient user and other components of the system—on a daily basis by a layperson user over an extended period comprising several or many consecutive days, unlike prior art mechanisms such as serum testing, laboratory-based tests, and/or tests that require a technician or healthcare professional. In an embodiment, the present inventor has recognized that the system may facilitate a more accurate, immediate and/or effective diagnosis of menopause than other mechanisms requiring interactions with healthcare professionals or technicians. In an embodiment, the present inventor has recognized that the diagnostic test 100, when performed on a daily basis at home, and when utilized in association with the Patient-Facing Application and Seed Consumption System as described elsewhere herein, may signal the specific days on which it is most beneficial to consume one or more specified types of seeds to facilitate hormone balancing, particularly beneficial to menopausal and perimenopausal women. In various embodiments, the diagnostic test 100 is useful in association with trend identification. In particular, the present inventor has recognized the benefit associated with the “at home,” disposable nature of the test in that it can be taken daily that allows for the identification of the trends associated with the undesirable absence of pregnanediol glucuronide as indicated by the stored results associated with any of the dates occurring from 7-10 days past the subject woman's ovulation date.

Diagnostic Test Key

Especially due to the possibility that the presence of color at one result indication line may indicate a positive result and the presence of color at a separate and distinct result indication line on the same strip may indicate a negative result, or vice versa, the present inventors have discovered that a Diagnostic Test Key, illustrated in one embodiment by FIG. 3, is useful in association with interpreting the results of the diagnostic test 100 in certain examples.

In an embodiment, the Diagnostic Test Key 200 comprises a printed card featuring a graphical depiction of each possible visual result of the diagnostic test, and what each possible visual result of the diagnostic test indicates, as depicted in FIG. 3. In an embodiment, the Diagnostic Test Key 200 comprises a digitized graphic, or a virtual representation generated in association with coded instructions, for utilization in association with the Patient-Facing Application described elsewhere herein and/or a digital reader 670 as described elsewhere herein. In an embodiment, the Diagnostic Test Key 200 comprises a digitized map of the correlation of the presence or absence of a specified color intensity (optionally represented by HEX or RGB codes) at a of specified location measured from one end of the lateral flow assay on a photographed lateral flow assay corresponding to the presence or absence of a hormone or analyte in a tested bodily fluid. In an embodiment, the Diagnostic Test Key 200 comprises multiple correlations of the presence or absence of a specified color intensity at a of specified location directed to a single lateral flow assay. In an example, configuration of the Diagnostic Test Key 200 comprising a digitized map may be made available to the Patient Facing Application in association with the other components of the system described herein and in accordance with computer application configuration mechanisms (i.e. code) as is well understood in the art. In an exemplary embodiment, a result indication line configured to provide a result for the presence or absence of PdG at a threshold in an applied fluid sample and a separate result indication line configured to provide a result for the presence or absence of LH at a threshold in an applied fluid sample is depicted in association with the Diagnostic Test Key, as shown on FIG. 3. In an example, the Diagnostic Test Key 200 depicts a diagnostic test 100 at least featuring a result indication line configured to provide an indication with regard to an applied fluid sample for the presence or absence of PdG at a threshold whereby the absence of a visual line, or the presence of a visual line below a specified color intensity, indicates the presence of PdG at the threshold in the applied fluid sample. In an example, the Diagnostic Test Key 200 depicts a diagnostic test 100 further comprising a result indication line configured to provide an indication with regard to an applied fluid sample for the presence or absence of LH at a threshold whereby the presence of a visual line, or the presence of a visual line above a specified color intensity, indicates the presence of LH at the threshold in the applied fluid sample. In an embodiment of the invention the Diagnostic Test Key 200 depicts at least one printed graphical representation of a diagnostic test 100 at a similar scale to the diagnostic test 100 with exemplary results depicted thereon alongside a verbal description of the exemplary results.

In an embodiment, the Diagnostic Test Key 200 is printed on to the same card as the instructions for use of the diagnostic test 100. In an embodiment of the invention, the Diagnostic Test Key 200 is digitally produced and incorporated into a Patient-Facing Application configured to utilize a processor to compare a photographed result to a the results indicated on a digitally reproduced Diagnostic Test Key. In an example the Diagnostic Test Key 200 is programmed in conjunction with the Patient-Facing Application. In an example, the Diagnostic Test Key 200 is coded for usage in association with a digital reader 670 configured for use in association with a diagnostic test 100 as described elsewhere herein. In an embodiment, the Diagnostic Test Key 200 is configured for use in association with a digital reader 670 also comprising a display 605 configured to depict a result and/or interpretation 607 of the test collected by directing a processor to compare the result digitally obtained by the test via a Diagnostic Test Key 200 and displaying the result on a display located on the screen. In an embodiment, the Diagnostic Test Key 200 is configured for utilization in coordination with the systems comprising digital readers 670 described in the following patent applications, hereby incorporated by reference: U.S. patent application Ser. No. 16/302,085 filed on May 19, 2017; PCT Patent Application PCT/US2019/038173 filed on Jun. 20, 2019; PCT/US2005/024422 filed on Jul. 8, 2005; U.S. patent application Ser. No. 10/888,676 filed on Jul. 9, 2004; and PCT/GB2014/052962 filed on Oct. 1, 2014, for example.

Healthcare Professional-Facing Application:

As utilized in association with the description of a “Healthcare Professional-Facing Application,” the term “application” is synonymous with a computer program. In an embodiment, the term “application” means a computer program designed to run on a mobile device, such as a smartphone. In alternative embodiments, the term “application” refers to a computer program designed to run on an alternative computing device such as a personal computer.

The Healthcare Professional-Facing Application in embodiments of the system is intended for usage by healthcare professionals. In various embodiments of the invention, the term “healthcare professional,” refers to a person who support implementations of health care, treatment and referral plans usually established by medical, nursing, respiratory care, and other health professionals, and usually require formal qualifications to practice their profession. In some circumstances, the term “healthcare professional” may also refer to an unlicensed assistive personnel assist with providing health care services as permitted or customary in their professional. In an embodiment of the invention, the Healthcare Professional-Facing Application is intended for use by healthcare professionals specializing in care associated with fertility or primary care. In various embodiments, the Healthcare Professional-Facing Application features access controls designed to limit the ability or means necessary to read, write, modify, or communicate data/information or otherwise use any system resource to a validated healthcare professional. In an embodiment, each healthcare professional is validated prior to providing access to the Healthcare Professional-Facing Application, optionally in association with identification cross-check or API access to the national provider identifier (NPI) database in accordance with log in and access control systems as well understood by those skilled in the art.

The Healthcare Professional-Facing Application in an exemplary implementation incorporates a healthcare professional profile. In an example, the healthcare professional profile is made accessible to a healthcare professional validated and logged into the Healthcare Professional-Facing Application (referred to herein as the “healthcare professional user”) in association with methods and mechanisms readily understood by those skilled in the art. The healthcare professional profile displayed to a healthcare professional user via a graphical user interface allows for the healthcare professional user to input information pertinent to their professional activities. In an example, the healthcare professional profile allows for the input by a healthcare professional user of licensure information, optionally including his or her one or more jurisdictions of licensure relevant to his or her professional services. In an example, the healthcare professional profile allows for the input by the healthcare professional user of identifying information, optionally including demographic information of the healthcare professional user, contact information of the healthcare professional user and/or the identification number of the healthcare professional user, such as the healthcare professional user's national provider identifier (NPI). In an example, the healthcare professional profile allows for the input by healthcare professional user of the conditions that the healthcare professional user has the capability to treat. The information is input by the healthcare professional in association with the Graphical User Interface, input/output mechanisms and/or operating system mechanisms associated with the Computing Device associated with the system, so configured via programming and or computer programming mechanisms as well understood by those skilled in the art.

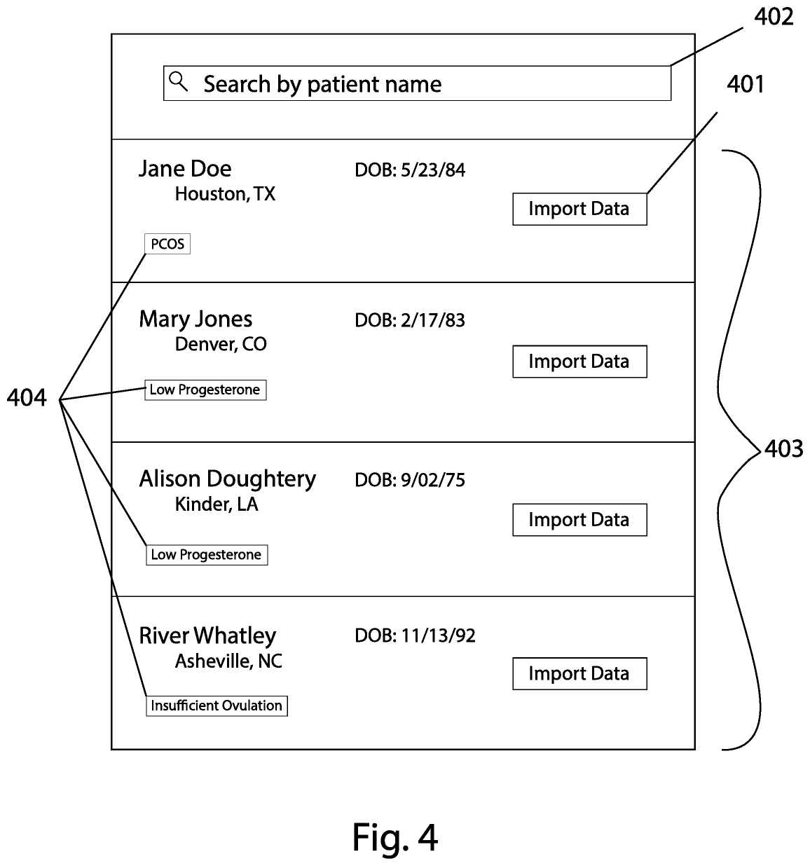

In the preferred embodiment, the Healthcare Professional-Facing Application features an element within its Graphical User Interface to allow a healthcare professional user to engage an ePHI importer/exporter 401 to export information relevant to any patient, any subset of patients and/or all patients that the healthcare professional is treating, has treated or intends to treat, as depicted in FIG. 4. In an example, the ePHI importer/exporter is configured to package any information relevant to a patient, optionally inclusive of diagnostic test results captured via a Patient-Facing Application, into an interoperable format, such as HL7, a clinical document architecture, a continuity of care document or continuity of care record, structured product labeling, clinical context object workgroup, a format relevant to the fast healthcare interoperability resources, a format relevant to the services aware interoperability framework, Arden syntax, formats associated with the Trusted Exchange Framework and Common Agreement and/or other similar interoperable format to allow the interoperable export of information relevant to a patient profile or plurality of patient profiles. In an example, the ePHI importer/exporter is configured to receive such information pertinent to a specific patient, optionally a patient for whom the healthcare professional has scheduled a telemedicine appointment as described elsewhere herein. In an example, the interoperable formatted ePHI is transmitted via the ePHI exporter to the healthcare professional user's electronic health records (EHR) system, or an EHR system of another healthcare professional to whom the healthcare professional user intends to refer one or more patients.

The Healthcare Professional-Facing Application incorporates a telemedicine block scheduling feature to facilitate the designation of certain time periods of the healthcare professional user as available periods to conduct a telemedicine appointment with a patient, optionally a patient user of a Patient-Facing Application. In various embodiments, the graphical user interface of the Healthcare Professional-Facing Application incorporates an element or elements configured to designate and store one or more specific time periods as an available period to conduct a telemedicine appointment with a patient. In various embodiments of the system, the designated and stored time period(s) are made available for subsequent viewing by one patient or a plurality of patients, each by utilization of the Patient-Facing Application via its graphical user interface as described elsewhere herein. In an embodiment, upon the selection of an available time period of the healthcare professional user by a patient user via the Patient-Facing Application, a notification to both the relevant patient user and the relevant healthcare professional user is generated containing and/or providing access to the relevant patient user's diagnostic test results and/or other electronic personal health information, optionally via a link to a profile depicting the patient user, and a link configured to initiate the telemedicine appointment by videoconference, VOIP call or phone call optionally as described elsewhere herein. In an embodiment of the invention, notifications via e-mail, SMS, delivery in association with an application operating on a smartphone (such as a “push notification”) or pre-recorded phone call are triggered to both the relevant patient user and the relevant healthcare professional user, optionally comprising a mechanism, optionally a hyperlink to a patient profile or the electronic medical record of a patient, to access diagnostic test results and other electronic personal health information of the patient user and a mechanism, optionally a hyperlink to an external application such as Zoom, Facetime, or a VOIP phone call conducted within the Graphical User Interface of the Healthcare Professional-Facing Application or the Patient-Facing Application, to trigger the telemedicine appointment. In various embodiments, the telemedicine system incorporates any of such technologies or other technologies in communications system to facilitate real-time discussion between the user and the healthcare provider during an appointment (or “virtual” appointment), as an aspect of the telemedicine system. The present inventor has recognized that such embodiment more efficiently allows for the facilitation of a telemedicine appointment at times convenient for both a patient user and a healthcare professional user with the patient user's relevant diagnostic test information made available to the healthcare professional user in an organized and consistent fashion in association with the scheduled appointment. In an embodiment, the telemedicine block scheduling feature comprises the Scheduler as described elsewhere herein.

It is a teaching of an embodiment of the Healthcare Professional-Facing Application to provide a graphical user interface element configured to search for by name and choose a patient user 402, optionally from a subset of patient users who have already selected a time for a telemedicine appointment or otherwise previously conducted a telemedicine appointment with the relevant healthcare professional user. The graphical user interface is configured to work in association with the other elements of the system as described elsewhere herein to display a variety of information related to the specifically chosen patient or list of patients 403, optionally including the display of patient diagnostic test results, display of patient demographic information, and the display of a suggested diagnosis of a medical condition relevant to the patient based on an evaluation of the patient's diagnostic test results as described elsewhere herein, an example of which is depicted in FIG. 4.

A teaching of an embodiment to display suggested results 404 of the one diagnostic test or plurality of diagnostic tests relevant to a specified patient to the healthcare professional user via the graphical user interface of the Healthcare Professional-Facing Application, optionally facilitated by sending the one or a plurality of results of one or a plurality of diagnostic test(s) relevant to a particular patient user to a clinical decision support system, via API connection the healthcare professional user's EMR or other healthcare systems, or via API connection to other databases configured to cross reference collected and/or interpreted diagnostic results to suggest medical conditions associated with such collected and/or interpreted diagnostic results.

Patient-Facing Application:

As utilized in association with the description of a “Patient-Facing Application,” the term “application” is synonymous with a computer program. In an embodiment, the term “application” means a computer program designed to run on a computing device, such as a smartphone. It is to be understood that the term “patient” when used in association with the terms “Patient-Facing Application” and/or “patient user” is a term of convenience and not necessarily literally intended to refer to or designate any user as a patient. Rather, the term “patient” in these contexts refers to persons that are not healthcare professionals, persons seeking health information or healthcare services, or persons not intended to use the associated features and components in the context of providing healthcare services. In alternative embodiments, the term “application” refers to a computer program designed to run on an alternative computing device such as a personal computer. In one embodiment, the Patient-Facing Application is a variant of the Healthcare Professional-Facing Application with a distinct subset of features and/or access limitations.

The Patient-Facing Application incorporates a patient profile. The patient profile displayed to a patient user via a graphical user interface allows for the patient user to input demographic information associated with the patient. In an example, the patient profile is made accessible to a patient validated and logged into the Patient-Facing Application (referred to herein as the “patient user”) in association with methods and mechanisms readily understood by those skilled in the art. In an example, the patient user consists of the subject woman of a diagnostic test configured to detect for at least the presence or absence of pregnanediol glucuronide at a specific threshold. Optionally, the patient may manually input other electronic personal health information or otherwise import or link to the patient's electronic personal health information, optionally by importing a continuity of care document or continuity of care record. In an example, the patient profile allows for the input of desired characteristics of healthcare professionals that the patient would like to interact with, optionally demographic information or jurisdictions of licensure. In an example, the patient profile allows for the input of the conditions that the patient seeks treatment for. In an example, the patient profile is populated with conditions associated with the patient automatically upon receiving and/or interpreting the results of diagnostic tests relevant to the patient. In an example, the results of the diagnostic tests 100 relevant to the patient are collected in accordance with other mechanisms of the system. In various embodiments, information is input into the patient profile by the patient in association with input output mechanisms and/or operating system mechanisms associated with the computer device associated with the system as well understood by those skilled in the art.

In the preferred embodiment, the Patient-Facing Application features an element to allow a patient to engage a ePHI exporter to export information relevant to that patient only. It is an aspect of the invention that the ePHI exporter can deliver the patient's electronic personal health information (ePHI) to a destination associated with a healthcare professional of the patient's choosing. In an example, the ePHI exporter when activated via the graphical user interface of the Patient-Facing Application packages any information relevant to a patient into an interoperable format, such as HL7, a clinical document architecture, a continuity of care document or continuity of care record, structured product labeling, clinical context object workgroup, a format relevant to the fast healthcare interoperability resources, a format relevant to the services aware interoperability framework, Arden syntax, formats associated with the Trusted Exchange Framework and Common Agreement and/or other similar interoperable format to allow the interoperable export of information relevant to that patient's profile. In an example, the patient profile provides an element to allow the patient to provide consent to release the relevant patient user's ePHI to one specific healthcare professional or a plurality of specified healthcare professionals.

In the preferred embodiment, the Patient-Facing Application is further configured to record and store the indicated result for the presence or absence of PdG at a pre-defined threshold and optionally the presence or absence of one or more additional hormones or hormone analytes at a pre-defined threshold of each diagnostic test 100 performed on a fluid sample of the patient. In various embodiments, it is a teaching of an embodiment for the system to instruct the patient user to conduct multiple diagnostic tests 100 each taken once every day for a number of consecutive days, optionally in association with the display of one or more interpretation(s) 607.

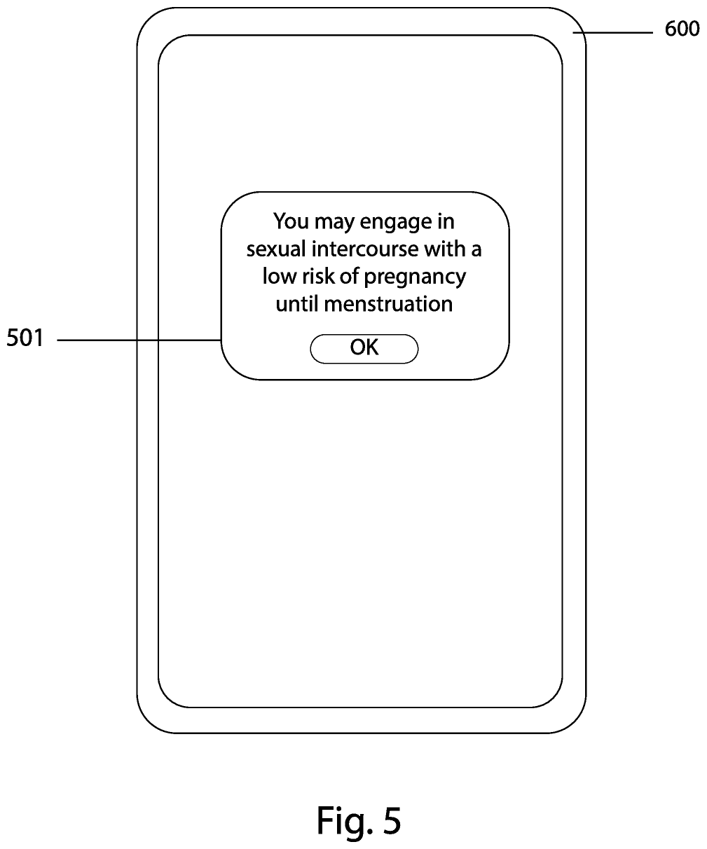

The Patient-Facing Application is optionally configured to display any of a variety of a limited subset of unique messages 501, an example of which is depicted by FIG. 5, each corresponding to an interpretation 607 of results indicated on a diagnostic test 100 captured in the Patient-Facing Application, following the generation of the interpretation 607 of the results in association with the Computing Device, Processor, Camera and/or other components as described elsewhere herein. The present inventor has recognized the unique advantages of the diagnostic test(s) 100 as described herein, particularly when utilized in association with the Patient-Facing Application, associated with the ability for the collection of results on multiple consecutive days, optionally during an extended time period, to assist with detecting changes, which may comprise increases, decreases, or trends, of levels of hormones and/or analytes over time. Such detected changes, particularly when the diagnostic test 100 is utilized in combination with a physical or digital (for example, when coded into the Patient-Facing Application) form of the Diagnostic Test Key 200 and/or Color Intensity Key 800, may include information related to the extent of the change, such as a 1.5-fold change or 2-fold change, optionally indicated and/or calculated in association with the color intensity displayed on the diagnostic test 100 following use. The present inventor has recognized that the specific messages generated, optionally corresponding to the interpretations 607 described herein, correspond to a subset of the specifically available and uniquely valuable interpretations 607 associated with each diagnostic test 100. In particular, due to the binary nature of at least a subset of the results generated by embodiments of the diagnostic test 100 comprising result indication lines, two of which are specifically configured to provide an indication for at least the presence or absence of LH at a threshold and the presence or absence of PdG at a threshold, respectively, in an applied fluid, the specifically available interpretations may be limited in an example to a subset comprising the below interpretations, or for each a similar unique message 501 with the same effect. In association with the Patient-Facing Application, each diagnostic test 100 in the preferred embodiment may be configured to evaluate for the presence or absence of any of FSH, an estrogen metabolite such as E3G, LH, PdG, or hCG, or any combination thereof, as described elsewhere herein. Each unique message 501 optionally derives from and depicts one or more of the following specific interpretations, optionally by utilizing the Processor, Patient-Facing Application or other component of the system as described herein, of each diagnostic test 100 or series of diagnostic test results collected daily over a plurality of consecutive days within a menstrual cycle. In the preferred embodiment of the invention, due to the limited applications, and more specifically due to unique combination of diagnostic tests configured primarily to provide a binary result related to either the presence or absence at a threshold of one or more of the hormones or hormone metabolites selected from the group consisting of LH, PdG and hCG and also configured to provide a semi-quantitative, quantitative or trend-based result (i.e. of a fold change) related to of one or more of the hormones or hormone metabolites selected from the group consisting of FSH and an estrogen metabolite (such as E3G), a specific subset of useful indications relevant to a woman's fertility status and/or menstrual cycle, and also specifically useful to embodiments of the invention described herein, may be derived. Such subset of interpretations, optionally comprising an indication or instruction each of which may be displayed as a unique message 501 in association with components of the invention further described elsewhere herein, and the trigger for display of such unique message 501, comprises:

Following a result of a 1.5-fold decrease in FSH within a single menstrual cycle, the interpretation comprising an indication that a follicle has been selected;
Following a result of a 1.5-fold decrease in FSH within a single menstrual cycle, the interpretation comprising an indication of the fertile window opening and the appropriate time to engage in intercourse for conception;
Following a result of a 1.5-fold decrease in FSH within a single menstrual cycle, or on the eighth day of the menstrual cycle (as the present inventor has determined that this is a suitable backup period to appropriately effectuate the continuance of the steps of the method if a FSH drop is not indicated), whichever occurs first, the interpretation comprising an indication that it is the appropriate time to commence testing for an estrogen metabolite such as E3G and an instruction to commence testing for an estrogen metabolite such as E3G;
Following a result of a 1.5-fold decrease in FSH within a single menstrual cycle, the interpretation comprising an indication that it is the appropriate time to discontinue testing for FSH and to commence testing for estrogen and an instruction to discontinue testing for FSH and to commence testing for estrogen;
Following a result of a persistently high level of FSH, the interpretation comprising an indication of the likelihood of that ovulation may not occur this cycle or a high risk of anovulation;
Following a result of a persistently high level of FSH, the interpretation comprising an indication of the likelihood of onset of menopause;
Following a result of a 1.5 fold increase in an estrogen metabolite, optionally E3G, within a single menstrual cycle, an interpretation comprising an indication that a follicle has matured;
Following a result of a 1.5 fold increase in an estrogen metabolite, optionally E3G, within a single menstrual cycle, an interpretation comprising an indication of the fertile window opening;
Following a result of a 1.5 fold increase in an estrogen metabolite, optionally E3G, within a single menstrual cycle, an interpretation comprising an indication of the follicle secreting estrogen;
Following a result of a 1.5 fold increase in an estrogen metabolite, optionally E3G, within a single menstrual cycle, an interpretation comprising an indication that it is the appropriate time to commence testing for LH and an instruction to commence testing for LH;
Following a result of a 1.5 fold increase in an estrogen metabolite, optionally E3G, an interpretation comprising an indication that it is the start of the fertile window and the appropriate time to engage in intercourse for conception;
Following a result of a persistently low level of an estrogen metabolite, optionally E3G, an indication that the subject woman will not ovulate during the menstrual cycle;
Following a result of a persistently low level of an estrogen metabolite, optionally E3G, an indication that the subject woman is likely not fertile during the menstrual cycle;
Following a result of the presence of LH at a threshold, an interpretation comprising an indication that ovulation is imminent;
Following a result of the presence of LH at a threshold, an interpretation comprising an indication of elevated fertility or peak fertility;
Following a result of the presence of LH at a threshold, an interpretation comprising an indication that the subject woman should engage in sexual intercourse to conceive;
Following a result of the presence of LH at a threshold, an interpretation comprising an indication that the it is the appropriate time to commence testing for PdG and an instruction to commence testing for PdG;
Following a result of the presence of LH at a threshold, an interpretation comprising an indication that the it is the appropriate time to commence testing for progesterone and an instruction to commence testing for progesterone;
Following a result of a persistently low level of LH, an interpretation comprising an indication that ovulation may not occur during this menstrual cycle;
Following a result of a persistently low level of LH, an interpretation comprising an indication of the likelihood that ovulation is insufficient in this menstrual cycle for the subject woman to conceive;
Following a result of the presence of PdG at a threshold on the days inclusive of 7-10 days past ovulation, an interpretation comprising an indication that the subject woman has sufficiently ovulated to conceive;
Following a result of the presence of PdG at a threshold, an interpretation comprising an indication that the infertile period has begun;
Following a result of the presence of PdG at a threshold, an interpretation comprising an indication that the subject woman may engage in sexual intercourse with a low risk of conceiving or pregnancy until the onset of menstruation in the subsequent menstrual cycle;
Following at least one result of the absence of PdG at a threshold on one of the days selected from the range inclusive of 7-10 days past ovulation, an interpretation the woman has not sufficiently ovulated;
Following a result of the presence of hCG at a threshold, an interpretation comprising an indication of pregnancy;
Following a result of the absence of hCG at a threshold, an interpretation comprising an indication that the subject woman is not pregnant; and
Following a result of the presence of hCG at a threshold and a result of the absence of PdG at a threshold, an interpretation comprising an indication that the subject woman has likely not produced enough progesterone to sustain pregnancy.

While the present inventor has specifically recognized the unique value of the above interpretations related to each diagnostic test 100 as described elsewhere herein, it is intended for the invention to optionally comprise additional interpretations, indications, instructions, prompts and unique messages 501 to more fully provide usefulness to the user of the embodiments described herein. Also, as referred to herein, any fold increase (i.e. 1.5 fold increase) or fold decrease (i.e. 1.5 fold decrease) as referred to herein in the context of the invention is considered to be at least that fold increase or fold decrease. For example, if a 3 fold increase for a hormone or analyte is indicated by a series of diagnostic tests, such indication also demonstrates a 1.5 fold increase. Also for example, if a 2 fold decrease is indicated for a hormone or analyte by a series of diagnostic tests, such indication also demonstrates a 1.5 fold decrease.

In various embodiments, the Patient-Facing Application generates a unique message 501, chosen from a series of unique messages optionally consisting of the message depicted in FIG. 5 or optionally another message, for display to the patient user via the graphical user interface, display, and/or other mediums of communication, such as e-mail, SMS, automated phone call or push notification via a smartphone graphical user interface as illustrated by FIG. 5. In various embodiments, the unique message 501 may comprise an alert to change the diagnostic test 100 to test for a different hormone and/or analyte. In an embodiment, the unique message 501 may prompt a user to record the date of onset of menstruation. In an embodiment, the unique message 501 may prompt a user to record a baseline test to facilitate comparison by subsequently performed diagnostic tests in the same cycle. In an embodiment, the unique message 501 may prompt a user to apply a sample of the user's urine to a diagnostic test 100.

The Patient-Facing Application in an embodiment is configured to aggregate the results of a plurality of diagnostic tests 100, each of which is performed daily by the patient user over a number of consecutive days for aggregation into a series to associate the results from each diagnostic test 100 taken daily during the period of the number of consecutive days with the patient user, optionally for transfer to a healthcare professional user via an ePHI importer/exporter. In the preferred embodiment, the diagnostic test 100 is performed by the patient user by applying first morning urine to the sample pad 191 (in an embodiment comprising the fluid application zone 106) of the diagnostic test 100. In one embodiment, the patient user performs the diagnostic test 100 on consecutive days during the period of 7-10 days past ovulation and utilizes the Patient-Facing Application to record the results of each diagnostic test 100, optionally in accordance with the teachings described in U.S. patent application Ser. No. 16/732,766 filed on Jan. 2, 2020, hereby incorporated by reference in its entirety with priority claimed thereto. In another embodiment, the system comprises a unique message 501 comprising instructions to the patient user to perform the diagnostic test 100 on consecutive days during the period of the patient user's single menstrual cycle and utilizes the Patient-Facing Application, in association with at least the camera, processor and computing device, to record the results of each diagnostic test 100. In various embodiments, it is a teaching of the system to utilize the data collected over multiple series to identify trends associated with the levels of PdG, and optionally additionally any of LH, an estrogen metabolite such as E3G, FSH, hCG and/or other tested hormone or hormonal analyte detected above or below a pre-defined threshold, or otherwise interpreted in association with a fold change as described elsewhere herein, associated with each diagnostic test 100.

In an embodiment, the application generates a unique message 501 to a user to prompt the user to initiate and utilize the application to evaluate a new diagnostic test 100 by utilizing the Patient-Facing Application on a daily basis. In the preferred embodiment, the relevant unique message 501 is generated in the morning to remind the user to utilize first morning urine in association with the diagnostic test 100, as opposed to a urine sample taken later in the day. The present inventor has recognized that in an example, LH tests can be performed up to 3 times per day due to short time period associated with a LH surge, in which case it is a teaching of an embodiment of the invention to incorporate enough diagnostic tests 100 to evaluate thrice daily, and to actually perform in association with steps referring to testing a fluid for LH, testing at times corresponding to first morning urine, mid-day, and in the evening. The present inventor recognizes that this is particularly useful in association with examples of the invention, as some studies show that a more precise measurement of LH in association with the diagnostic test 100 configured to measure LH at a threshold is performed in the evening, as LH is higher in the evening in some women.

In various embodiments, the storage may exist on the mobile computing device itself or via a communicatively connected storage device, such as, for example, cloud connected storage. The application is configured to present a graph of the results over a time series on the display of the smartphone 600 or other computing device.

The Patient-Facing Application in an embodiment is configured to utilize the results detected by one or more diagnostic tests 100, optionally within a series of diagnostic tests performed daily during the time period correlating to a menstrual cycle, to detect a trend or trends of hormonal concentrations from one menstrual cycle to at least one other menstrual cycle or a plurality of other menstrual cycles. The trends are thereby interpreted by the Patient-Facing Application, and optionally delivered to a healthcare provider via a Healthcare Professional-Facing Application and/or the telemedicine system each described elsewhere herein, to generate suggested treatment protocols. In an example, a suggested treatment protocol comprises progesterone supplementation following the persistent absence of PdG as indicated on a series diagnostic tests 100 as further described elsewhere herein. In another example, a suggested treatment protocol comprises the consumption of a specified amount of pumpkin and flax seeds, optionally incorporated within a single consumable food item, optionally in snack bar form, commencing upon the first day of menstrual bleeding in a menstrual cycle. In another example, a suggested treatment protocol comprises the consumption of a specified amount of sesame and sunflower seeds, optionally incorporated within a single consumable food item, optionally in snack bar form, following the indication of the presence of LH at a threshold as indicated on a diagnostic test 100, as further described elsewhere herein. It is therefore a teaching of the method embodiment to perform the step of utilizing the results detected by one or more diagnostic tests to detect a trend or trends of hormonal concentrations from one menstrual cycle to at least one other menstrual cycle 8080. It is therefore a further teaching of the method embodiment to perform the step of interpreting the trends to generate suggested treatment protocol 8081, in association with the Patient-Facing Application and optionally by accessing clinical decision support systems via API as described elsewhere herein. In a method embodiment, it is a teaching of an embodiment to perform the step of suggesting a treatment 8082 optionally in the form of a suggested treatment protocol, and optionally in accordance with the teachings of the seed consumption system related to the consumption of certain seeds as described elsewhere herein or the ingesting, supplementation or injection of progesterone, optionally delivered via a display 605 or the graphical user interface of the Patient-Facing Application. It is a further teaching of the method embodiment to perform the step of delivering the suggested treatment protocol to a healthcare provider 8083. In a method embodiment, the suggested treatment protocol generated relates to the consumption of certain seeds as described elsewhere herein. It is a further teaching of the method embodiment to perform the step of supplementing progesterone following the identified trend of the persistent absence of PdG at a threshold over a time period as indicated by a series of diagnostic tests performed on a fluid of a subject woman 8085. Following the step of identifying of a trend persistent absence of PdG by either the Patient-Facing Application pre-configured to identify and detect such a trend associated with a subject woman, or by a healthcare provider identifying and detecting such a trend associated with a subject woman optionally in association with a telemedicine consultation as described elsewhere herein, which is a teaching of a method embodiment of the invention, it is a further teaching of a method embodiment to perform the steps of purchasing a progesterone supplement or plurality of progesterone supplement doses and delivering the progesterone supplement or plurality of progesterone supplement doses to the subject woman 8084. In various embodiments, clinical decision support systems or artificial intelligence technologies are utilized to generate treatment suggestions based on the detected trends of hormonal concentrations. In a method embodiment, it is a further teaching of an embodiment to perform the step of delivering the suggested treatment to one or more healthcare professional user(s), optionally (a) healthcare professional user(s) that a patient user has initiated an appointment with via the Scheduler 8086. In various embodiments, and in part due to the recurring nature of the menstrual cycle and the associated necessary testing, the steps described above are either repeated or performed in a variety of different orders. The present inventor recognizes the need to change the subset of steps or order in which the steps are performed or repeat steps or a subset of steps in certain examples, as the results of hormonal levels associated with the menstrual cycle may overlap at various and unexpected points in the menstrual cycle.

In a method embodiment, the Patient-Facing Application utilizes the detected hormonal levels or trends of hormonal levels as collected by a series of diagnostic tests to generate suggestions of diet changes applicable to the patient user. In an embodiment, the suggested treatment protocol, optionally associated with the “suggesting” step described in the preceding paragraph, consists of generated suggestions of diet changes. For example, the ingestion of certain seeds, due to their chemical compound composition, is well known to affect the hormone levels of a person who ingests such seeds, and it is useful to correlate the timing and/or quantity of consumption of such seeds to the results of one or more diagnostic tests 100 as described herein. In an embodiment, optionally in association with the “suggesting” step described above, the application is configured to generate suggestions for the consumption of certain seeds at specified times to affect hormonal concentrations of the patient user. In a method embodiment of the invention, it is a further teaching to perform the step of prompting the purchase of one or more products containing the suggested amount of seeds to ingest. In an example, the prompting takes place in the form of a subscription. In an example, the photographed diagnostic test 100 is shown within the graphical user interface of the Patient-Facing Application following the generation of a suggestion for the consumption of certain seeds at specified times. In an embodiment, the Patient-Facing Application is configured to facilitate the purchase by the patient user of the one or more products containing the suggested amount of seeds to ingest at a time for delivery to the patient user prior to the suggested specified time. The Patient-Facing Application triggered delivery to the home or other desired location by the patient user is orchestrated in an example via FedEx, UPS, Amazon or other logistical and delivery service as well understood by those skilled in the art.

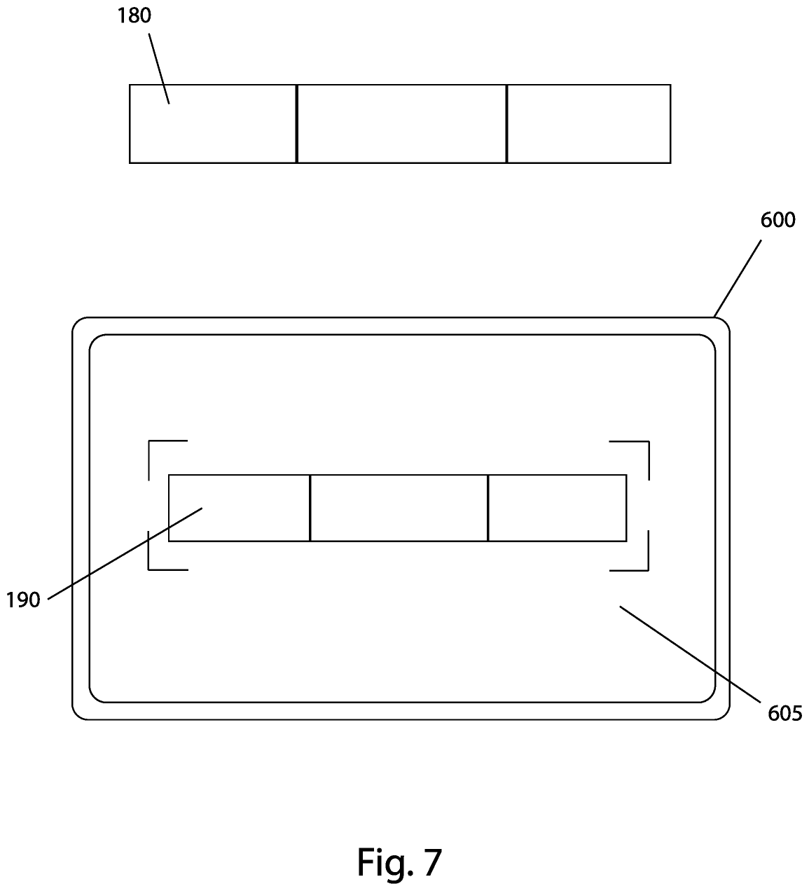

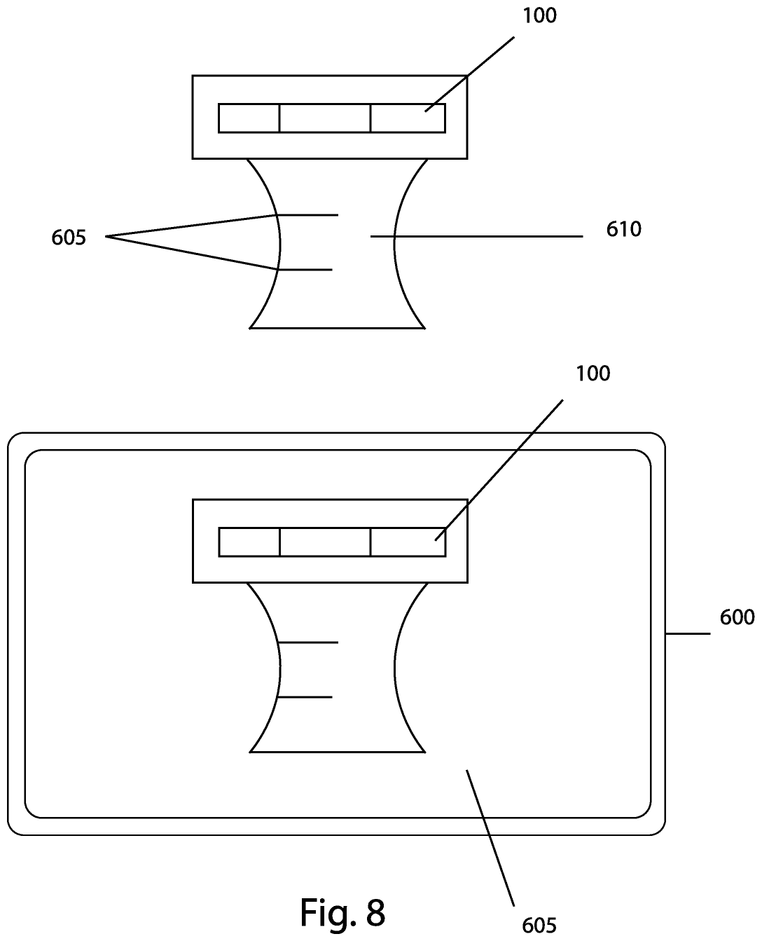

In an embodiment, the Patient-Facing Application further comprises a diagnostic test capture tool, illustrated by FIG. 7. The diagnostic test capture tool is configured utilize the display of a smartphone 600 to facilitate the alignment of the diagnostic test 100 within a smartphone 600 display for photographing and interpreting the results of a detected diagnostic test 100. In various embodiments, diagnostic test 100 comprises a lateral flow assay comprising at least a testing zone configured to detect for the presence or absence of PdG at a threshold, the threshold optionally selected from the range inclusive of 1 μg/mL-10 μg/mL, as further described elsewhere herein. In embodiments, the diagnostic test 100 comprises multiple testing zones, each configured to evaluate for the presence or absence of a single hormone or analyte, wherein one testing zone is configured to evaluate for the presence or absence of PdG at a threshold selected from the range inclusive of 1 μg/mL-10 μg/mL, as further described elsewhere herein.

In an embodiment, the diagnostic test capture tool further comprises a stand 610 configured to hold a diagnostic test 100 in position during capture of a photograph in association with the Camera and the display 605 of the smartphone 600. In an embodiment, the stand 610 comprises markings 615 of a specified distance apart to aid in the calculation of the dimensions of the diagnostic test 100 held by the stand 610. In an embodiment, the interpretation of results of the diagnostic test 100 held by the stand 610 occurs in accordance with the teachings elsewhere herein associated with the specific sequence of result indication lines each corresponding to a specific testing zone each configured to evaluate for the presence of a specific hormone and/or analyte. In an embodiment, the interpretation of results of the diagnostic test 100 held by the stand 610 occurs in accordance with the teachings elsewhere herein associated with the specific distance of each result indication line from one end of a diagnostic test 100, optionally calculated by a Processor detecting the markings 615 on a stand 610 photographed by the smartphone 600 and comparing the pre-determined distance between the markings 615 to the diagnostic test 100 held by the stand 610 to assist with the determination of the specific dimensions of the diagnostic test 100, optionally by counting the specific number of pixels contained in a straight line between the markings 615 and then allocating the pre-determined distance between the markings to that number of specific pixels to allocate a specific width to a pixel, and subsequently counting the number of pixels and allocating the specific width to each pixels to determine a result for the distance between the photographed edge of a diagnostic test 111 and one or more of the first testing zone, second testing zone, third testing zone, fourth testing zone, first result indication line 107, second result indication line 108, third result indication line 109, and fourth result indication line 110, to determine the location of each testing zone and/or result indication line and interpret the indicated result for the presence (or optionally the presence or absence at a threshold) of a specified hormone or analyte associated with each testing zone and corresponding result indication line. In an example, the Patient-Facing Application works in association with the diagnostic test 100 capture tool to facilitate the capture of the diagnostic test 100 and the interpretation of results. Such results may then be utilized by the Processor and other components of the system to provide information to the graphical user interface related to the results, including treatment strategies and/or suggested treatment protocols, optionally comprising diet changes, optionally in accordance with the teachings and methods described elsewhere herein.

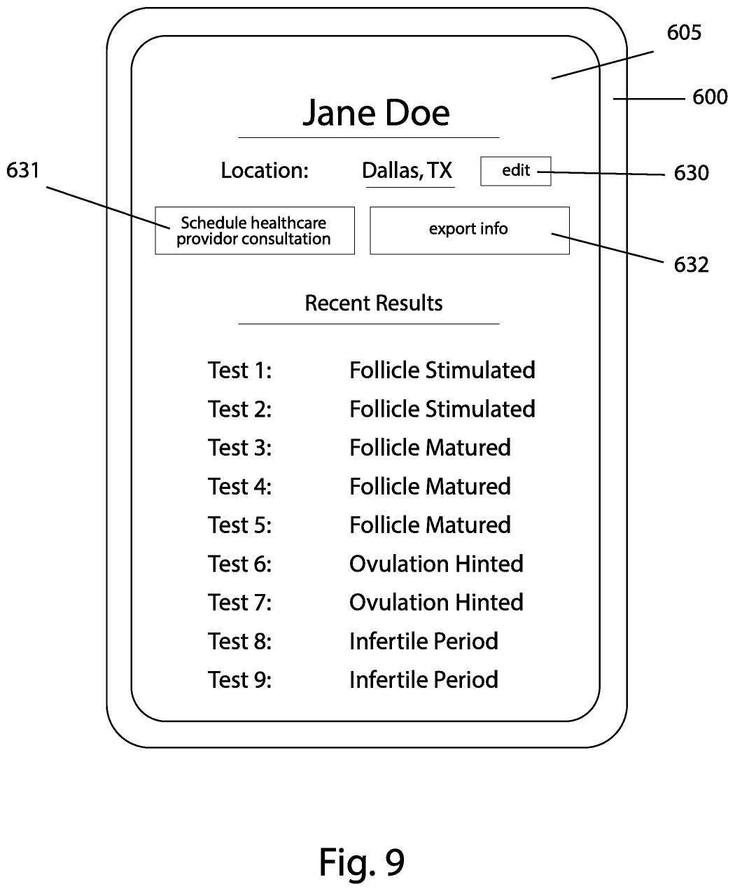

In various embodiments, the Patient-Facing Application incorporates the Telemedicine System as described elsewhere herein, and is thus configured to allow a patient user to access available appointment times of healthcare professionals capable of treating medical conditions that may be suggested by the results of a diagnostic test or series of diagnostic tests captured and interpreted via the Patient-Facing Application. The Patient-Facing Application in an example incorporates a graphical user element configured to allow the patient user to set the patient's jurisdiction of residence 630, optionally within a graphical user interface as illustrated in FIG. 9. In an embodiment, the Patient-Facing Application is configured to facilitate the operation of the Telemedicine System within its graphical user interface, for instance via a graphical user interface element configured to allow a patient user to schedule a healthcare provider consultation 631, and optionally via a separate graphical user interface element configured to export information derived from the patient's collected diagnostic test results 632 as depicted on FIG. 9, which may include the packaging and delivery of results of one or more diagnostic tests 100 collected via the Patient-Facing Application in association with other components of the system and/or other electronic personal health information to one healthcare professional or a plurality of healthcare professionals in an interoperable format in accordance with the teachings elsewhere herein. In an example, the Patient-Facing Application is further configured to receive and distribute results from a sexual partner, optionally the results of a sexual partner's sperm test or plurality of sperm tests, and optionally in association with information triggered for export by the patient user as described herein, for further interpretation by a healthcare professional.

In various embodiments, the Patient-Facing Application incorporates the Seed Consumption System as described elsewhere herein, and is correspondingly configured to provide suggestions of the consumption of certain seeds and/or products containing certain seeds based on the interpreted results of a diagnostic test or series of diagnostic tests captured and interpreted via the Patient-Facing Application. In an embodiment, the Patient-Facing Application is configured to facilitate the operation of the Seed Consumption System described elsewhere herein within its graphical user interface. In various embodiments, the Patient-Facing Application is configured to provide suggestions, optionally in the form of suggested treatment protocols, for the consumption and/or supplementation of progesterone of a specific amount, optionally in droplet format and optionally in association with the triggering of a purchase and delivery of the progesterone, in response to a detected indication for the absence of pregnanediol glucuronide generated in association with a diagnostic test as described elsewhere herein. In various embodiments, such suggestions are delivered to a patient user each as a unique message 501 as illustrated by FIG. 5. As such, it is a step in a method embodiment engage in suggesting supplementation of progesterone, and optionally triggering the purchase of a progesterone supplement or plurality of progesterone supplement doses, in response to a detected indication for the absence of pregnanediol glucuronide on a diagnostic test. In an embodiment, the supplementation of progesterone occurs in coordination with a progesterone supplement system.

An embodiment of the invention comprises a progesterone supplement system. The progesterone supplement system comprises a plurality of diagnostic tests configured to detect for the presence or absence of LH at a threshold as defined herein, and a plurality of diagnostic tests configured to detect for the presence or absence of PdG at a threshold as defined elsewhere herein. The progesterone supplement system further comprises a plurality of progesterone supplement doses. In the preferred embodiment, each progesterone supplement dose contains a quantity of progesterone selected from the range of 25-35 mg. In the preferred embodiment, each dose is formulated in an oil suspension. An exemplary oil for use in association with the oil suspension is coconut oil or MCT oil. In the preferred embodiment, the oil comprises mixed tocopherols (vitamin E). The present inventor has recognized that by formulating the progesterone with vitamin E in the form of a progesterone supplement dose, the vitamin E acts as a carrier to deliver the progesterone directly into the blood stream and quickly. In one example, the steps of formulating each progesterone supplement dose include adding 5-10% weight by volume of Vitamin E (also referred to as “mixed tocopherols”) with 90-95% weight by volume on MCT oil or coconut oil. Subsequently, perform the step of dissolving the bio-identical progesterone (natural progesterone), such that the final concentration of progesterone should be 7-15 grams per 100 ml. Optionally, perform the step of heating the mixture of the preceding sentence at 60-70 C for 5-20 minutes to increase solubility.

The present inventor by testing various steps has likewise determined a preferred method of progesterone supplementation in association with the progesterone supplement doses. In the preferred method, a user engages in the step of testing, on a daily basis, for the presence or absence of LH in a bodily fluid at a threshold via a diagnostic test as defined elsewhere herein 3450. Following the first result, indicating the presence of LH at a threshold in the tested bodily fluid, optionally commencing at a time 2 days after the first result indicating the presence of LH at threshold in the tested bodily fluid, the user (optionally by instruction generated in association with the Patient-Facing Application, a digital device 670 or other display 605 following the first result indicating the presence of LH at a threshold) engages in applying a progesterone supplement dose containing 25-35 mg of progesterone formulated in an oil suspension to the membranes of the mouth three times for a period of at least 7 days or the period until the start of the next menstrual cycle, whichever is longer 3451. The present inventor has determined the enhanced effectiveness of this form of progesterone supplementation, particularly in comparison to other forms of over-the-counter progesterone supplementation, in that progesterone applied in this manner is better absorbed into the blood stream. The method may further comprise steps of testing, during the period of 7-10 days past ovulation, for the presence or absence of PdG in the bodily fluid with a diagnostic test comprising a testing zone and corresponding result indication line configured to detect for PdG at a threshold selected from the range of 1 μg/mL-10 μg/mL 3452; and Applying a quantity of 75-105 mg of progesterone formulated in an oil suspension to the membranes 193 of the mouth on a daily basis during the subsequent menstrual cycle if any of the diagnostic tests comprising a testing zone and corresponding result indication line configured to detect for PdG and provide a result indicating the absence of PdG on any of the days during the period of 7-10 days past ovulation (optionally by instruction generated in association with the Patient-Facing Application, a digital device 670 or other display 605 following a result indicating the absence of PdG at a threshold in an applied fluid during the timeframe of 7-10 days past ovulation of the subject woman) 3453.

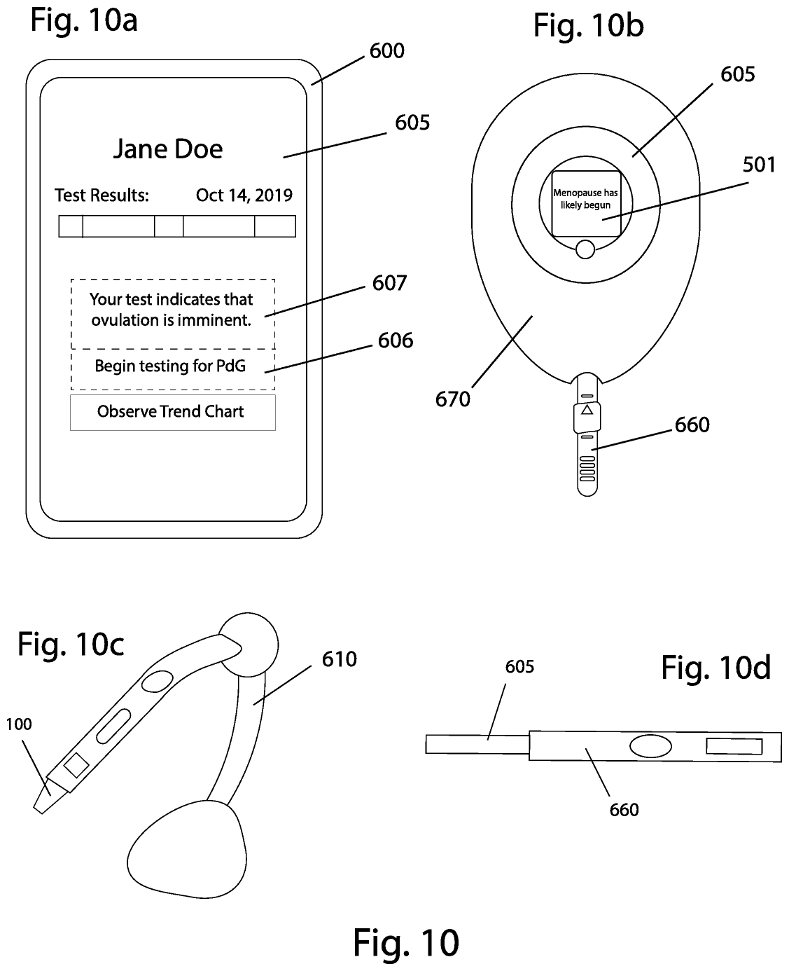

In various embodiments, an interpretation 607 deriving from the result of a diagnostic test 100 as described elsewhere herein, optionally comprising an interpretation 607 collected in association with the Fertility Tracking System and/or methods of use of the Fertility Tracking System as described elsewhere herein, optionally collected in association with the Patient-Facing Application, is displayed to the user via the display 605, optionally the display 605 associated with the Patient-Facing Application operating in association with a smartphone 600. An example of such an interpretation 607 is depicted in FIG. 10a. In various embodiments, a prompt 606 or plurality of prompts in association with the Fertility Tracking System and/or methods of use of the Fertility Tracking System as described elsewhere herein is delivered to a patient user via the graphical user interface of the Patient-Facing Application, optionally each as a unique message 501. An example of such a prompt 606 depicted in association with a diagnostic test result and interpretation 607 is depicted in FIG. 10a. In various embodiments, a prompt 606 or other aspects of the Patient Facing Application are configured for delivery to a display 605 integrated within a cartridge containing a lateral flow assay 660 as illustrated by FIG. 10d, or the display 605 of a digital reader 670 configured to evaluate a lateral flow assay by placement of a cartridge containing a lateral flow assay 660 therein as illustrated by FIG. 10b, optionally in association with the other electronic components of the cartridge containing a lateral flow assay 660 and/or the digital reader 670.

### Computing Device

In varying embodiments of the invention, a computing device is useful in capturing, processing and storing the results indicated by one or more diagnostic test(s), along with the demographic information of a specified subject woman and associated suggested treatment protocols, configured as described elsewhere herein.

In the preferred embodiment, the computing device consists of a smartphone 600. In various embodiments, the term “smartphone” is defined as a mobile phone that performs many of the functions of a personal computer, typically having a touchscreen interface, internet access, and an operating system capable of running downloaded applications. A smartphone 600 may be defined more broadly as a mobile telecommunications device. In various embodiments, the smartphone 600 consists of either an Apple iPhone or Google Android device. In various embodiments the smartphone 600 is configured to operate a version of the iOS or Android operating systems. In one embodiment, the computing device is operated in association with an application, optionally the Patient-Facing Application further configured to incorporate mechanisms to control, collect data from or otherwise interact with the computing device. In an alternative embodiment, the computing device consists of a personal computer.

In an alternative embodiment, the computing device comprises a server or communicatively other connected computer accessed via the internet via a smartphone 600 or local personal computer. In such embodiment, the Patient-Facing Application or Healthcare Professional-Facing Application may be operated by a patient user, optionally consisting of a subject woman whose bodily fluid has been applied to at least one diagnostic test 100 as referred to elsewhere herein, or a healthcare professional user as applicable via a web browser in accordance with mechanisms and methods well understood by those skilled in the art.

In an exemplary embodiment, the computing device consists of the system described in U.S. patent application Ser. No. 16/302,085 filed on May 19, 2017, incorporated by reference herein. In another exemplary embodiment, the computing device consists of the system described in PCT Patent Application PCT/US2019/038173 filed on Jun. 20, 2019, incorporated by reference herein.

In the preferred embodiment, the computing device incorporates and/or controls storage, at least one processor and at least one camera. In various embodiments, the Patient-Facing Application and Healthcare Professional-Facing Application are each configured to operate upon the operating system of the computing device in accordance with mechanisms and procedures well understood by those skilled in the art. In various embodiments, the computing device comprises a cloud system configured to communicatively connect with a smartphone 600 for interpretation and analysis of the data collected from a diagnostic test 100 in association with a photograph of the diagnostic test 100 taken and interpreted by the smartphone 600 as more comprehensively described elsewhere herein.

Camera

In association with the invention, it is useful to provide a camera configured to collect the results of the diagnostic test. This is especially the case when recording an image of a diagnostic test via a computing device operated by a layperson in association with the systems and methods as described elsewhere herein. In an embodiment, the camera consists of a camera integrated into a smartphone 600 as is well understood in the art. In alternative embodiments, the camera is incorporated into a cartridge also housing a diagnostic test 100, or in a digital reader 670 configured to receive and interpret a diagnostic test 100 placed within the digital reader 670.

In the preferred embodiment, the camera of the system is incorporated within a smartphone 600 or tablet computer operating the Patient-Facing Application. In an embodiment, the system comprises a smartphone 600, mobile telecommunications device, or tablet computer featuring both a rear-facing or front-facing camera. It is the intent of the inventor that any camera of a smartphone 600 configured with multiple cameras may be utilized in association with the system as described herein.

In the preferred embodiment, the camera associated with the system is utilized to photograph the lateral flow assay, the processor associated with the system is utilized to interpret the color within each of a plurality of result indication lines on the lateral flow assay, the processor associated with the system is configured to interpret the intensity of the color within each of a plurality of result indication lines on the lateral flow assay to determine a result, the result is associated with the date the camera photographed the lateral flow assay, the result is stored in the communicatively connected storage medium, and optionally displayed in a calendar format via a graphical user interface. In accordance with such teaching, it is a further teaching of a method embodiment to perform the step of determining a result 2019. In an example, the determining a result step is performed by photographing, via a camera integrated within a smartphone 600, the diagnostic test 1000, optionally comprising a lateral flow assay test. In an example, the Patient Facing Application is pre-configured to identify, optionally by identifying the shape of the diagnostic test 100 depicted in a photograph captured by the camera, and measuring the distance from one end 111 or both ends of the diagnostic test 100, and in association with the Processor and distances that the Patient Facing Application is pre-configured to associate with one or more result indication line(s) each configured to evaluate for the presence or absence of a distinct hormone or analyte at a threshold. The collection of a photograph in an optimal fashion is optionally by utilization of an alignment mechanism displayed within the display of a smartphone 600 as depicted in FIG. 7 to allow a user of the Patient Facing Application to align the test in a useful manner to aid in the determination of the distance to and identification of each result indication line on the lateral flow assay to be photographed. In one example, the user is prompted to align one end 111 of the diagnostic test 100 with an element in the graphical user interface to facilitate the identification and measurement of the diagnostic test 100.

It is therefore a teaching of a method embodiment to perform the step of orienting the photograph of the lateral flow assay test to determine the locations of the one or more result indication lines of the lateral flow assay test 2020. It is also a further teaching of a method embodiment to perform the step of identifying the specific color intensity (optionally by RGB or HEX color code), optionally with the assistance of the color intensity key 800, associated within the color detected by the camera within a result indication line of the lateral flow assay test 2021. It is a further teaching to perform the step of comparing the color intensity associated with the color detected by the camera within the result indication line of the diagnostic test 100 with a pre-defined threshold intensity signifying the presence or absence of the hormone or hormonal analyte 2022 associated with the result indication line in the applied fluid. As further described in more detail elsewhere herein, the lateral flow assay test referred to in such step optionally consists of a single test configured to simultaneously or near-simultaneously detect for the presence or absence of a plurality of hormones or hormonal analytes selected from the group consisting of pregnanediol glucuronide, luteinizing hormone, an estrogen metabolite such as E3G, FSH, estradiol, progesterone and human chorionic gonadotropin 2023.

In an embodiment of the invention, the term “camera” refers more broadly to an optical sensor. An optical sensor of the disclosure can have several components, including 1) a raw camera sensor; 2) LED lights; 3) a microcontroller; 4) an aperture; 5) a shutter; and 6) a simple optical lens. For example, an optical sensor of the disclosure can have an optical system comprising of a fluid (e.g. poly(dimethylsiloxane) (PDMS)) or solid (e.g., glass) material lens and a complementary metal-oxide semiconductor (CMOS) or a charge-coupled device (CCD) image sensor. The optical sensor can also use an orientation element located on the lateral flow device to locate the one or more result indication lines and the control line. An optical sensor in an embodiment comprises a high-resolution camera configured to take an image of at least one result indication line and the control line of the diagnostic test 100. For example, the optical sensor can be a countertop device, a stand-alone device, the optical sensor module as described in PCT Patent Application PCT/US2019/038173 filed on Jun. 20, 2019, incorporated by reference herein, the “detection instrument” as described in U.S. patent application Ser. No. 16/302,085 filed on May 19, 2017, incorporated by reference herein, or a smartphone camera. In varying embodiments, the optical sensor may be contained within an apparatus intended for use apart from the diagnostic test 100, such as the digital reader 670 depicted in FIG. 10b. In another embodiment, the optical sensor is contained within an apparatus sharing a containment mechanism with the diagnostic test 100, such as a cartridge 660, as depicted in FIG. 10d.

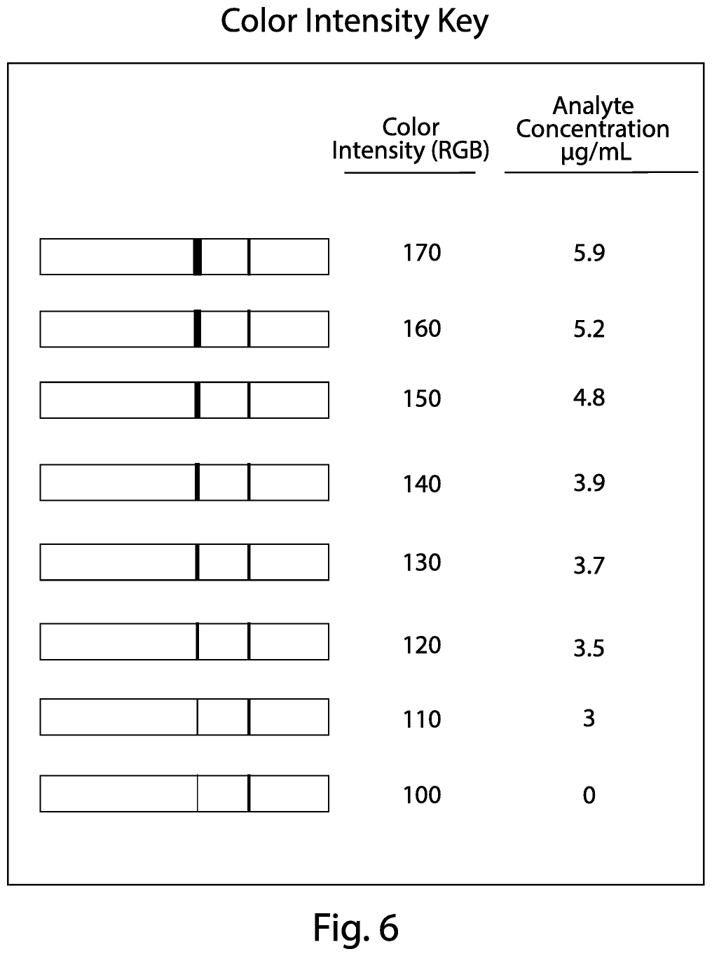

In an example of the invention, the camera is configured to collect one or more optical signals each originating from a distinct result indication line of a diagnostic test 100, optionally in coordination with the processor. In one embodiment, the camera is configured to receive a plurality of optical signals each originating from one of a plurality of result indication lines in coordination with mechanisms to measure the length to and sequence of each result indication line and match the result indicated by each result indication line, optionally via a Diagnostic Test Key, as described elsewhere herein. In an embodiment, the camera is configured to work in conjunction with the processor and/or other elements of a system to determine an amount of at least a first analyte, optionally the presence or absence of the first analyte at a threshold, and a second analyte, optionally the presence or absence of the second analyte at a threshold, in a biological sample applied to the fluid application zone 106 based on said optical signals, wherein an optical signal associated with the first result indication line 107 increases with decreasing amounts of said first analyte present in said biological sample, and an optical signal associated with the second result indication line 108 increases with increasing amounts of said second analyte present in said biological sample. In an embodiment, the RGB or HEX color codes associated with specific quantities of a hormone or hormone analyte are determined prior to use of the diagnostic test 100, optionally by applying spiked male urine containing pre-measured quantities of a hormone or hormone analyte as described elsewhere herein, and the RGB or HEX color codes associated with each indication for each quantity are recorded, optionally in association with the color intensity key and/or the diagnostic test key 200. In association with various embodiments, in FIG. 6, the “R” values associated with the “RGB” color codes are depicted as an example. In association with computer application mechanisms well-known in the art (such as, for instance, the color matching tools associated with Adobe Photoshop and other graphic editing applications, or components thereof), the quantity of a hormone or hormone analyte (or the presence or absence of a hormone or hormone analyte) is determined by finding the closest RGB color code or HEX color code to the RGB color code or HEX color code pertaining to the color evident on a result indication line following the application of a fluid to a diagnostic test 100, and then estimating the quantity of the relevant hormone or hormone analyte or the presence or absence of a hormone or hormone analyte based upon the pre-measured quantity of the hormone or hormone analyte associated with said closest RGB color code or HEX color code, optionally as indicated on the color intensity key and/or diagnostic test key 200.

In an example, the camera may operate in coordination one or more light sources forming a part of the disclosure for illuminating the diagnostic test 100 or at least the first result indication line 107 of the diagnostic test 100 configured to detect for at least the presence or absence of pregnanediol glucuronide at a threshold selected from within the inclusive range of 1 μg/mL-10 μg/mL, or as further described in U.S. patent application Ser. No. 16/732,766 filed on Jan. 2, 2020, hereby incorporated by reference with claim of priority made thereto.

Communicatively Connected Storage Medium

Generally, the term “storage” as referred to herein refers to any technology used to place, keep and retrieve electronic data. “Communicatively connected storage medium” as referred to herein, refers to a medium within a computing device, such as a solid-state drive contained within a smartphone 600, but may alternatively refer to cloud storage in which data is transmitted and stored on remote storage systems, where it is maintained, managed, backed up and made available to users over a network (typically the internet) as is well understood by those skilled in the art. It is a teaching of the system that the electronic personal health information, photographs of one or more diagnostic test(s) 100, results of one or more diagnostic test(s) 100, and/or any information relevant to a healthcare professional user or a patient user is stored within storage, which in an example consists of one or more communicatively connected storage medium(s).

Processor

As used herein, the singular term “processor” or plural term “processors” generally refer to the electronic circuitry within a computing device that executes instructions that make up a computer program and/or application as well understood by those of skill in the art. In an example, the processor consists of a cloud computing mechanism operating in conjunction with a smartphone as is well understood by those skilled in the art. In an example, the processor consists of one or more processors of a smartphone 600 utilized in association with the system described herein.

In association with the various systems and methods described herein, the diagnostic test 100 configured to evaluate urine for the presence or absence of at least pregnanediol glucuronide at a threshold selected from the inclusive range of 1 μg/mL-10 μg/mL comprises at least the first result indication line 107 configured such that, optionally when used with a base unit, a first optical signal (e.g., a fluorescent signal) is capable of being detected at the first result indication line 107. In various embodiments, the location of the first result indication line 107 and/or one or more other result indication lines is determined by a pre-defined sequence made available for use by the system, by a measurement of the diagnostic test 100 to determine the pre-programmed location of each result indication line as described elsewhere herein. The first optical signal may be a readout for the amount of pregnanediol glucuronide in the sample, for example, by detecting the amount of first detection reagent, in an example comprising the colloidal gold-labeled PdG antibody, bound to the first capture reagent, in an example comprising the PdG-BGG conjugate, by correlating the color intensity of the first optical signal developed to a pre-determined measurement of the level of pregnanediol glucuronide correlating to the color intensity. It is a teaching of an embodiment to determine the resulting color intensity(ies), and/or the resulting RGB color code(s) or HEX color code(s) associated with each evident color, of the first result indication line 107 collected from a plurality of diagnostic tests at a time selected from the range inclusive of 3-20 minutes following the application of samples each containing a distinct, fixed amount of pregnanediol glucuronide to create a color intensity key 800, optionally comprising a part of or useful in association with the Diagnostic Test Key 200, for uses in association with those described herein. In one example, the average or median color intensity and/or RGB or HEX color code is determined by sampling a plurality of pixels contained within one result indication line to collect the result, optionally for incorporation the Diagnostic Test Key 200 and/or the color intensity key 800 to associate a specific color intensity, RGB color code or HEX color code with a quantity of hormone or hormone analyte present in the diagnostic test 100. In one example, the average or median color intensity and/or RGB or HEX color code is collected for each of a plurality of tests, and then the average or median color intensity and/or RGB color code or HEX color code is then determined for the plurality of tests to determine a result, optionally for incorporation the Diagnostic Test Key 200 and/or the color intensity key 800 to associate a specific color intensity, RGB color code or HEX color code with a quantity of hormone or hormone analyte present in the diagnostic test 100. An exemplary color intensity key 800 is depicted by FIG. 6, though it is to be understood that in varying configurations a single color intensity key 800 may provide associations with multiple color intensity combinations, whereby each color intensity optionally associated with a specific sequence or location coordinate as determinable by calculating the distance of the indication from one end 111 of a diagnostic test 100, correlating to multiple hormones and/or analytes. A similar protocol is likewise used for one or more result indication line(s) configured to evaluate alternative hormones and analytes. It is a teaching of an embodiment in the preferred embodiment to utilize only diagnostic tests 100 in association with the methods described herein that were manufactured according to standardized manufacturing protocols in a similar manner to those utilized to create the color intensity key 800. In accordance with the determination of the correlation of different levels of color intensity with a specific level of pregnanediol glucuronide, each of the plurality of diagnostic tests similarly configured to a diagnostic test 100 intended for subsequent real-world use is applied with a sample of male urine, each containing a specified quantity of precisely measured added amount of pregnanediol glucuronide. In various embodiments, each such sample may be referred to as a male urine sample spiked with pregnanediol glucuronide. The intensity of each such sample is measured and optionally re-measured, and the average color intensity displayed across all diagnostic tests for which a male urine sample spiked with a standardized amount of pregnanediol glucuronide is associated with the standardized amount of pregnanediol glucuronide in association with evaluation purposes. It is therefore a teaching of a method embodiment of the invention to create a color intensity key 800 by the steps of creating a sample of fluid containing a known amount of a hormone or analyte 8090, applying the sample of fluid to a diagnostic test 100 configured to evaluate for the presence of the hormone or analyte 8091, measuring the color intensity indicated following the application of fluid 8092, allocating the color intensity indicated to the known amount of the hormone or analyte 8093, and optionally aggregating the plurality of color intensity indications each allocated to a known amount of hormone or analyte into a color intensity key 8094. It is a teaching associated with an embodiment that the phrase “color intensity” as referred to herein may also refer to or correspond to a specific RGB color code or HEX color code. The present inventor has recognized the usefulness of the step of utilizing a plurality of color intensity keys each correlating to a specific hormone or analyte in a distinct result indication line in a specific diagnostic test 100 configuration. The color intensity key facilitates the ability to determine one or more results from a diagnostic test 100, as the color intensity key applied in conjunction with the predetermined distances of each result indication line and/or the sequence of the hormone and/or analyte tested within each result indication line, determinable on a photographed diagnostic test 100 in association with the teachings as described elsewhere herein, facilitates the identification of both the hormone and/or analyte tested and the amount of the hormone and/or analyte indicated within the relevant result indication line. In an embodiment, the sequence of hormones/and or analytes associated with each result indication line is made available for evaluation in association with the relevant color intensity key to determine the amount of hormone and/or analyte precisely associated with each result indication line of the diagnostic test 100. In an embodiment, the color intensity key 800, and the sequence of hormones/and or analytes or the distance of each result indication line and the associated hormone and/or analyte of each result indication line, are programmed into the Patient-Facing Application or a digital reader 670 to facilitate the detection of each color intensity indicated within a result indication line on the diagnostic test 100. The Patient-Facing Application in an example is also configured to interpret of the results of a diagnostic test 100 by selecting the closest matching color intensity on the color intensity key 800 for each detected indication within each of the result indication lines, and deriving the previously determined amount of hormone or analyte correlating to that closest matching color intensity as associated to the hormone or analyte associated with the relevant result indication line.

The color intensities associated with their correlated standardized amounts of pregnanediol glucuronide, optionally aggregated into a color intensity key 800, may be displayed on the Diagnostic Test Key for utilization in association with interpreting the diagnostic test 100 in an example. In an example, the color intensity key 800 is utilized in association with a processor configured to compare the color intensity of a photographed diagnostic test with the closest color intensity indicated on the color intensity key 800 correlated to a previously determined amount of pregnanediol glucuronide correlating to the closest color intensity as to provide an estimation of the amount of pregnanediol glucuronide. In an example, the processor is configured to evaluate a photographed diagnostic test 100 to which a fluid sample has been applied to compare the color intensity of the evident color within a result indication line of that diagnostic test 100 to the color intensity that the result indication line would exhibit at the threshold associated with that result indication line, to aid in determining a result for the presence or absence of the relevant hormone or hormone analyte at the threshold. It is a teaching of an embodiment to measure the color intensity of the relevant result indication line of a diagnostic test 100 configured to evaluate urine for the presence or absence of pregnanediol glucuronide following the application of a male urine sample spiked with pregnanediol glucuronide in a specific amount selected from the inclusive range of 1 μg/mL-10 μg/mL (also referred to as the “Threshold Concentration”), and set the measured color intensity as the Threshold Color Intensity for the interpretation of similarly manufactured diagnostic tests, and optionally indicate the threshold color intensity on a color intensity key 800 and/or a diagnostic test key 200 for subsequent testing purposes. The present inventor notes that the Threshold Concentration and Threshold Color Intensity is only relevant in various configurations to the result indication line configured to detect for the presence of pregnanediol glucuronide, and specifically not necessarily relevant to other result indication lines configured to detect for the presence of other hormones or hormone metabolites. In the preferred embodiment, a diagnostic test 100 is configured with a first result indication line 107 having the Threshold Color Intensity, wherein when the first result indication line 107 exhibits a color intensity less than the Threshold Color Intensity (for example, by the evident absence of a visually perceptible line), the associated interpreted indication is that of a positive result for pregnanediol glucuronide at the Threshold Concentration.

It is a teaching of the preferred embodiment of the invention to include a processor configured to operate in conjunction with the other elements of the system as described herein configured to evaluate a diagnostic test 100 to which a fluid sample has been applied to compare the color intensity of at least the first result indication line 107 with the Threshold Color Intensity, and then based on the comparison, determine a result for the presence or absence of pregnanediol glucuronide at the Threshold Concentration and then optionally display the result via a graphical user interface or as a unique message 501 as described elsewhere herein.

In various embodiments, the color intensity of one or more separately collected optical signals collected from the same diagnostic test 100 provides an indication or a plurality of indications for the presence or absence of one or more additional hormones and/or analytes. In such embodiments, the processor is configured in accord with the teachings herein to utilize the one or more separately collected optical signals to interpret an additional result or additional results indicated by the diagnostic test 100. In the preferred embodiment, the color intensity of the optical signal obtained from the first result indication line 107 configured to analyze for the presence or absence of pregnanediol glucuronide at a threshold increases in intensity when the amount of pregnanediol glucuronide present in the sample is lower, and such optical signal decreases in intensity when the amount of pregnanediol glucuronide present in the sample is higher. In an example, the optical signal obtained from within the first result indication line 107 configured to analyze for the presence or absence of pregnanediol glucuronide is inversely proportional to the amount of pregnanediol glucuronide in the fluid sample applied to the diagnostic test 100 containing the first result indication line 107. In various aspects, the diagnostic test 100 further comprises at least a second result indication line 108 configured to produce an optical signal likewise corresponding to the presence or absence of a second analyte or hormone at a threshold, optionally luteinizing hormone (LH), wherein the optical signal obtained from within the second result indication line 108 configured to analyze for the presence or absence of luteinizing hormone is directly proportional to the amount of luteinizing hormone in the fluid sample applied to the diagnostic test 100 containing the second result indication line 108. In various aspects of the systems and methods herein, the diagnostic test 100 is configured for utilization in conjunction with a base unit or digital reader 670 as described elsewhere herein and in various applications incorporated by reference herein, together comprising a system embodiment.

In one example, a system is provided comprising: a housing, comprising:

a) a port for receiving an diagnostic test 100, the diagnostic test 100 comprising two or more result indication lines each corresponding to a testing zone of the conjugate pad 190, one result indication line of which is configured to provide an indication of the presence or absence of pregnanediol glucuronide at a threshold of a specific amount selected from the inclusive range of 1 μg/mL-10 μg/mL;
b) a reader, comprising:
i) one or more light sources for illuminating said two or more result indication lines;
ii) one or more light detectors, optionally consisting of a camera or cameras as described elsewhere herein, configured to detect optical signals from each of the two or more result indication lines; and
c) a data analyzer comprising one or more processors configured to receive the optical signals in association with other components of the system as described elsewhere herein and to determine for the presence or absence of pregnanediol glucuronide at the threshold of a specific amount selected from the inclusive range of 1 μg/mL-10 μg/mL and a second analyte or hormone present in a biological sample applied to the diagnostic test 100 based by evaluating the optical signals, wherein the optical signal obtained from within the result indication line configured to evaluate for the presence or absence of pregnanediol glucuronide decreases with increasing amounts of pregnanediol glucuronide present in a biological sample applied to the diagnostic test 100, and an optical signal obtained from within a second of said two or more result indication lines increases with increasing amounts of said second analyte present in the biological sample.

An exemplary system may include a housing for containing components as described elsewhere herein, optionally configured as illustrated in FIG. 10b. The housing can be constructed of any suitable material. The housing may be configured to receive a lateral flow assay configured to detect for at least the presence or absence of pregnanediol glucuronide as described elsewhere in the disclosure. The housing may include a port or opening for receiving a diagnostic test 100, optionally contained within a cartridge. The system may further include, optionally contained within the housing, a reader device. The reader device in an embodiment comprises the camera as described elsewhere herein and the processor configured to interpret the results of the diagnostic test 100 as described elsewhere herein. The reader device may include one or more light sources for illuminating the diagnostic test 100 or a first result indication line 107 configured to detect for at least the presence or absence of pregnanediol glucuronide. In one particular example, the one or more light sources are calibrated to generate a light wavelength suitable to illuminate a detectable label, optionally a fluorescent label or colloidal gold, providing an indication of whether pregnanediol glucuronide is present or absent at a threshold within the first result indication line 107. In a particular example, the detectable label provided on the immunoassay device is a fluorophore, and therefore, the one or more light sources of the reader device should include a fluorescent light source (e.g., a light-emitting diode (LED)). It is to be understood that the wavelength of light provided by the light source of the reader device should be selected based on the excitation wavelength of the detectable label, and can readily be selected by a person of skill in the art. In an embodiment, the reader may be configured to illuminate the both the first result indication line 107 configured to provide an indication for the presence or absence pregnanediol glucuronide in a sample applied to the diagnostic test 100, and a second result indication line 108, each at a wavelength of light calibrated to accurately obtain the optical signal from each label, optionally at separate wavelengths. In some cases, the reader is configured to scan across the diagnostic test 100, comprising a test strip of an immunoassay device. In such cases where the immunoassay device utilizes a single fluorophore, the reader may contain a single fluorescent light source. In cases where the immunoassay device utilizes more than one fluorophore, the reader may contain more than one fluorescent light source. In various embodiments, the processor is configured to interpret the optical signals obtained from within each result indication line and discern among the wavelengths to generate a result. In various embodiments, the interpretation is made with the assistance of the color intensity key 800, wherein the color intensity key 800 is pre-configured to indicate the presence of specified quantities of hormones and/or analytes following the application of fluorescent light from the fluorescent light source to the immunoassay device utilizing at least one fluorophore based upon the evident color intensity(ies).

The reader may further comprise one or more light detectors (e.g., a photodetector) for detecting optical signals from the diagnostic test 100. Generally speaking, the one or more light detectors should be capable of distinguishing between emitted light at a first discrete position and a second discrete position on the diagnostic test 100. This may be accomplished by, e.g., the one or more light sources scanning across the diagnostic test 100 and determining the position of the emitted light on the diagnostic test 100.

In an embodiment, it is a teaching of an embodiment to provide a data analyzer. The data analyzer may have one or more processors configured to receive an optical signal. In some cases, the data analyzer is in operable communication with a reader device, optionally as described in as described in PCT Patent Application PCT/US2019/038173 filed on Jun. 20, 2019, incorporated by reference herein, the “detection instrument” as described in U.S. patent application Ser. No. 16/302,085 filed on May 19, 2017, incorporated by reference herein. In various examples, the reader device is programmed to utilize the color intensity key 800 to interpret detected results. The data analyzer may be configured to determine an amount of analyte or hormone present in an applied fluid sample, for example, by measuring the intensity of an optical signal obtained from within a result indication line of a diagnostic test 100 configured to detect for the presence or absence of pregnanediol glucuronide, optionally in association with the color intensity key 800. For example, the data analyzer may be configured to calculate the area under the curve of a signal intensity plot. The data analyzer may further be configured to determine the differences between signal intensities among the multiple discrete result indication lines or regions on the diagnostic test 100, each optionally providing an optical signal deriving from a different wavelength of light. In an example, the data analyzer may be configured to determine the difference between the signal intensity at the first result indication line 107 of a diagnostic test 100 and the signal intensity at the second result indication line 108 to provide a result. In an example, each result is collected by evaluating the difference in intensity between the optical signal within a result indication line and the optical signal collected from another aspect of the diagnostic test 100 and determining whether the difference in color intensity exceeds a threshold. In an embodiment, to accomplish a teaching in the previous sentence, the difference of color intensity between the control line 105 and a result indication line of a separate diagnostic test of a similar configuration following the application of a male urine sample spiked with pregnanediol glucuronide at a threshold is measured to provide a threshold difference for subsequent use, optionally for use in association with a Diagnostic Test Key 200 and/or color intensity key 800. The threshold difference then previously obtained from the separate diagnostic test 100 of a similar configuration is compared by the data analyzer to determine any difference in a diagnostic test 100 to which a fluid sample is applied for testing, and if the difference in color intensity of the diagnostic test 100 to which a fluid sample is applied exceeds the threshold difference, then a positive result for pregnanediol glucuronide in the applied sample is determined by the data analyzer in the embodiment. In the case that the difference does not exceed the threshold difference in the embodiment, a negative result for pregnanediol glucuronide in the applied sample is determined by the data analyzer. In various embodiments, the data analyzer then generates and transmits the result to other components of the system, optionally including the display 605, by mechanisms as readily understood by those skilled in the art. The data analyzer may further be configured to calculate an amount or concentration of the analytes present in the sample by similar mechanisms.

In an embodiment, the data analyzer may be further configured to detect a binary optical pattern. The binary optical pattern can be generated by two fluorescent materials which excitation and/or emission spectrum differs in wavelength. In some cases, the binary optical pattern can be generated by one fluorescent material and one light absorbent material. The detection reagents may be conjugated with the two types of materials respectively and can be captured in the same result indication line, such that the result indication line may generate two different optical signal patterns in the data analyzer.

In various aspects, the system may comprise a housing 670 for containing the processor and/or other electronic components, such as those depicted in FIG. 10b. The encasement of FIG. 10d may also be characterized as a housing for purposes in accordance with the teachings herein. The housing, in an example consists of a top housing and a bottom housing. The top housing in an example comprises a display 605 for indicating the results of the diagnostic test 100, as depicted in FIG. 10d, providing an indication at least for the presence or absence of pregnanediol glucuronide, said indication obtained by mechanisms as described elsewhere herein. The system and/or its processor may further comprise a display cover. The system may further comprise a battery. The system and/or its processor in an embodiment comprises a circuit board containing electronic components.

The system and/or its processor in an embodiment further comprises an optomechanics module. The optomechanics module in an embodiment comprises the one or more light sources and one or more light detectors as described elsewhere herein. In varying embodiments, the optomechanics module comprises the optical sensor module as described in PCT Patent Application PCT/US2019/038173 filed on Jun. 20, 2019, incorporated by reference herein, or the “detection instrument” as described in U.S. patent application Ser. No. 16/302,085 filed on May 19, 2017, incorporated by reference herein. The optomechanics module is configured in an embodiment as comprising the one or more light sources for illuminating the diagnostic test 100 configured to detect for at least the presence or absence of progesterone or a progesterone analyte. The optomechanics module in an embodiment is movable across an optical axis such that the optomechanics module moves laterally across the diagnostic test 100 to detect for at least the presence or absence of pregnanediol glucuronide by enabling alignment with the relevant result indication line of the diagnostic test 100. The system may further comprise an actuation module. The actuation module may comprise one or more motors configured to actuate/move the optomechanics module. In some embodiments, the motors may be coupled to a rack and pinion mechanism that is configured to translate the optomechanics module along one or more directions. For example, the optomechanics module can be translated along a longitudinal axis of the diagnostic test 100. The direction(s) of translation may or may not be orthogonal to an optical axis of the optomechanics module. In varying embodiments, the optomechanics module comprises, contains or is communicatively linked to the camera as described elsewhere herein. The direction(s) of translation may be parallel to the longitudinal axis of the diagnostic test 100, and the optical axis may be orthogonal to the longitudinal axis or a planar surface of the diagnostic test 100. In some cases, the direction(s) of translation need not be parallel to the longitudinal axis of the diagnostic test 100, and the optical axis need not be orthogonal to the longitudinal axis (or a planar surface) of the diagnostic test 100. For example, the direction(s) of translation and/or the optical axis may be at an oblique angle relative to the longitudinal axis of the diagnostic test 100.

In various aspects, the system and/or its processor may include an optical configuration suitable for use with the diagnostic test 100 and positioning of the optics above a result indication line configured to detect for at least the presence or absence of pregnanediol glucuronide. The optical configuration may include a light source (e.g., a light-emitting diode (LED) for illuminating the diagnostic test 100. The optical configuration may further include one or more lens, a filter, a optical beamsplitters, or any combination thereof. The optical configuration may further include a photodetector for detecting an optical signal from the diagnostic test 100. In an example, the system is configured to an excitation/emission spectra with an excitation wavelength of 492 nm and an emission wavelength of 512 nm.

In an embodiment, the processor is configured to perform an evaluation on the diagnostic test 100 to obtain a result by comparing the color intensity indicated on the diagnostic test 100 to at least one color intensity associated with a color intensity key 800 to derive the closest color intensity and retrieve the associated hormone or analyte concentration. This may be accomplished, for example, by utilizing the camera to calculate and detect the distance of one or more result indication line(s) from the proximal end of the diagnostic test by matching the dimensions with pre-determined diagnostic test 100 proportions, and detecting the evident color intensity(ies) within the result indication line(s) contained in the diagnostic test 100 at specified locations by comparing the evident color intensity(ies) to the color intensity(ies) included within the color intensity key 800 and/or the Diagnostic Test Key 200.

For instance, the processor may be configured to detect the dimensions of the diagnostic test 100 via a camera in association with a preconfigured known height of the diagnostic test 100. In an example the processor is configured to extrapolate the measurements of the length of the diagnostic test 100 by normalizing the height of the diagnostic test 100 as photographed to a pre-determined height and performing the appropriate mathematical equations (for example, the Pythagorean equation) to determine the length of the diagnostic test 100, and in particular to determine the length distance from one end 111 to one or more result indication line(s) located on the diagnostic test 100 and the length distance from the proximal end to the control line 105 located on the diagnostic test 100. In an example, the processor is configured to evaluate for to detect the color intensity evident within one or more result indication line(s) located at a predetermined distance or distances from the one end 111 of the diagnostic test 100 in a predetermined sequence, and compare the color intensity evident within each result indication line(s) to the closest known color intensity with an associated known concentration corresponding to a quantity of the relevant hormone or hormonal analyte to determine the indicated result or results, the indicated result or results optionally indicating the presence or absence of one or more hormones and/or hormonal analytes at a threshold. The processor is then configured to store and display the indicated result or results in coordination with the other inventive elements as described herein.

In various embodiments, the processor is communicatively linked to the other components of the system to collect and transmit signals and/or the indicated result(s) to the other components as needed to enable functioning of the system as is well understood by those skilled in the art.

Display

Various embodiments of the system as described herein comprise a display 605. In the preferred embodiment, the display 605 consists of the screen of a smartphone 600 or a tablet computer. In various embodiments, the display 605 consists of a screen incorporated within a cartridge, as depicted in FIG. 10d. In various embodiments, the display 605 consists of a screen incorporated within a digital reader 670 or base unit, as depicted in FIG. 10b. In embodiments of the invention, the display is configured to make visible a graphical user interface to a user. In various embodiments of the invention, the display is integrated with pressure-sensitive digitizers, such as the Apple Force Touch system. It will be widely understood to those skilled in the art that information displayed within and elements visible on the display 605 in certain embodiments may be manipulated or otherwise interacted with by a user via a variety of input output devices, optionally including a touchscreen, keyboard, keypad, mouse or other input output devices as well understood in the art. In various embodiments, the display 605 comprises the display as described in PCT Patent Application PCT/US2019/038173 filed on Jun. 20, 2019, incorporated by reference herein, or the display as described in U.S. patent application Ser. No. 16/302,085 filed on May 19, 2017, incorporated by reference herein. In an embodiment, the display is configured as a small screen placed within a housing or cartridge also containing a diagnostic test 100 such as that illustrated by FIG. 10d.

Graphical User Interface

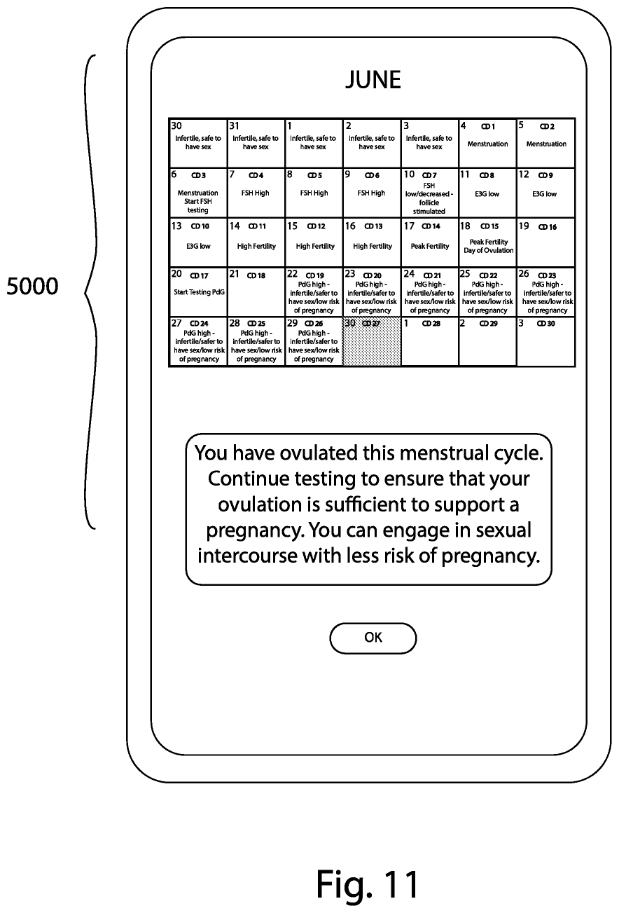

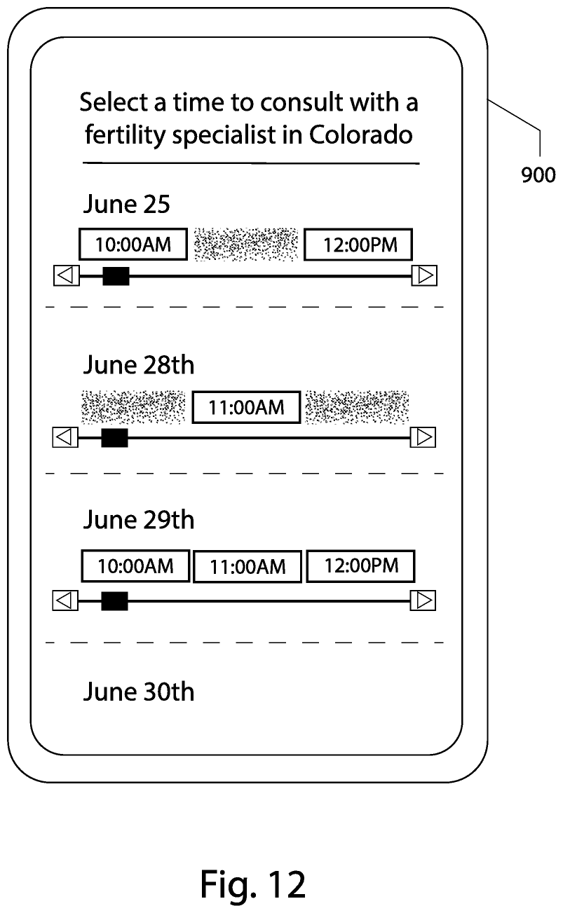

In varying examples the system comprises a graphical user interface. The graphical user interface is configured in accordance with mechanisms as well understood by those in the art to operate on a display 605. The processor may the graphical user interface to present one result or a series of results, and/or one or a series of unique message(s) 501 to a user within a graphical user interface, optionally via the Patient-Facing Application, such as is illustrated by FIG. 9. In an example, results and/or one or a series of unique message(s) 501 are also presented to a separate user via a graphical user interface operating in conjunction with the Healthcare Professional Facing Application. In varying embodiments, the graphical user interface is configured to operate on a smartphone 600, a personal computer, a tablet, or within a web browser operating on another such device. In a particular example, the results for each diagnostic test 100 displayed within a graphical user interface featuring the depiction of a calendar 5000, with the result, interpretation and/or unique message 501 associated with each diagnostic test 100 previously performed presented in association with the date each diagnostic test was performed within a calendar 5000 displayed similar to that as depicted in FIG. 11. In an embodiment, the various phases of the menstrual cycle, for instance menstruation, the follicular phase and the luteal phase, are indicated by color coding the days associated with each phase as indicated based on the results or indications of one or more diagnostic test(s) 100. In another particular example, a listing of available times to consult with a healthcare professional located in the same jurisdiction as the detected location of the user 900 of the Patient-Facing Application, optionally detected by utilizing the GPS of the smartphone 600, displayed in order of available dates starting with the nearest available date within a graphical user interface operating in conjunction with the Patient-Facing Application, as depicted in FIG. 12.

Calendar

The preferred embodiment of the invention comprises a calendar 5000. In an example, the calendar 5000 is configured for use in association with the collected results, each result from one of a plurality diagnostic tests, and the depiction of the collected results within a graphical user interface associated with the Patient-Facing Application as shown in FIG. 11. The present inventor has recognized the unique usefulness of organizing the collected result(s), along with associated indication(s) and interpretation(s), of a series of diagnostic tests for depiction in a calendar format, in part due to the cyclical nature of the menstrual cycle, in part due to the ease by which specific trends are observable within a calendar format, and in part due to the ability to identify specific phases of the menstrual cycle and group certain messages associated with the menstrual cycle by color coding periods of consecutive days within a depicted calendar, which comprises a teaching of an embodiment. The calendar 5000 configured for depiction within a graphical user interface associated with the Patient-Facing Application may depict the result(s), indication(s), or interpretation(s) of each diagnostic test 100, or a relevant unique message 501, collected for a single person on a daily basis on each date depicted within the calendar 5000. In an exemplary embodiment, each result, or category of results, for positive or negative is represented by a different color as depicted on the date of the collected diagnostic test result. In an exemplary embodiment, each of the phases of the menstrual cycle and/or the fertile and infertile timeframes of a menstrual cycle as interpreted by the collected series of diagnostic test results, is represented by a different color depicted over a series of days on the depicted calendar 5000. In an exemplary embodiment, each date is labelled with text identifying the hormone or analyte for which the result and/or interpretation is derived from as illustrated by FIG. 11.

The calendar 5000 depicted within a graphical user interface in an embodiment is configured to display a positive or negative result for each diagnostic test 100 on the date which the test was collected. In an embodiment, each interpretation of each diagnostic test 100 is represented by a different color and associated with the date of the diagnostic test was performed in the calendar 5000 in the graphical user interface. In accordance with the foregoing, it is a teaching of an embodiment and a step of the method of use of the system to engage in allocating the result from a diagnostic test consisting of a lateral flow assay test configured to detect for the presence or absence of at least pregnanediol glucuronide in urine to the specific calendar date upon which the result was determined 2002. It is also a teaching of and step of the method of use of the system to engage in storing the result and the calendar date associated with the result 2003, in coordination with other components of the system, including the Communicatively Connected Storage Medium, as described herein. It is also a step of the method of use of the system to engage in displaying the results of a diagnostic test via the graphical user interface 2008. It is likewise a step of the method of use of the system to engage in displaying the results of a diagnostic test via the graphical user interface featuring a calendar with the result of each test displayed on in association with the date each test was performed within the displayed calendar 2025. The present inventor has recognized that such a configuration allows for the easy identification of hormonal trends associated with the menstrual cycle. The present inventor has also recognized that such teaching representing an aspect of the invention in an embodiment is a solution to the confusion faced by a lay user of a diagnostic test with regard to interpreting an indicated result, particularly when a single diagnostic test 100 is configured to evaluate for the presence or absence of multiple hormones and/or analytes within a single sample of urine simultaneously. The present inventor has recognized that such problem is especially poignant, and the relevant solution that the foregoing represents is especially valuable, when the diagnostic test 100 lacks plain language text labelling on a readable surface to distinguish between the result indication lines and/or visual indications of the diagnostic test 100.

A calendar 5000 similar to that configured for depiction within the graphical user interface associated with the Patient-Facing Application may also be configured for depiction within the Healthcare Professional-Facing Application when a user of the Healthcare Professional-Facing Application is accessing the information relevant to one subject person.

In an embodiment, the Patient-Facing Application may be configured to utilize the calendar 5000 and more specifically its association of dates to the specific diagnostic tests performed to interact with a Patient Information Integration Tool associated with the invention. The purpose of the Patient Information Integration Tool is to associate the interpreted result of the diagnostic test and the time that the interpreted result was collected with the patient's demographic information and optionally other results associated with the patient. In an embodiment, the Patient Information Integration Tool may comprise an application programming interface (API) configured to facilitate the incoming and outgoing information formatted in an interoperable format, such as HL7 or others described in the Fast Healthcare Interoperability Resources specification, as is understood by those skilled in the art. In various embodiments, the Patient Information Integration Tool is configured as the examples described in U.S. patent application Ser. No. 14/997,503 filed on Jan. 16, 2016, U.S. patent application Ser. No. 12/391,120 filed on Feb. 23, 2009; U.S. patent application Ser. No. 16/113,652 filed on Aug. 27, 2018; and U.S. patent application Ser. No. 15/862,837 filed on Jan. 5, 2018, each of which are incorporated by reference. In an embodiment, the calendar 5000 is configured to collect and depict information associated with a specific date from other electronic medical record (EMR) systems and other healthcare modalities via the Patient Information Integration Tool. In an embodiment, the Patient Information Integration Tool is configured to display alerts optionally in association with the calendar 5000, in an example as described in U.S. patent application Ser. No. 16/743,029 filed on Jan. 15, 2020, which is hereby incorporated by reference. In an example of the invention, the calendar 5000 is configured to display elements of a treatment plan collected from external sources optionally based in part on the results of the diagnostic test, for example as is described in more detail in U.S. patent application Ser. No. 15/596,356 filed on Oct. 8, 2019 which is hereby incorporated by reference. Also in accordance with such teachings, in various embodiments, the method of use of the system further comprises the step of formatting the result from the diagnostic test, optionally comprising a lateral flow assay test, in combination with individually identifying information associated with the subject woman and the date the test was performed into formatted results for interoperable transfer to a computing device configured to interpret and store electronic personal health information 2011, optionally facilitated in association with the Processor, the Computing Device, the Patient Facing Application and/or the Patient Information Integration Tool as described herein. Likewise, it is a further teaching of the method of use of the system to provide a step for facilitating the transfer of the result in an interoperable format either individually or in combination with one or more additional results associated with the same subject woman 2012, optionally facilitated in association with the Patient Information Integration Tool.

In various embodiments, each diagnostic test result is collected in association with other aspects of the invention as described elsewhere herein. Each diagnostic test result may be correlated with a date provided or otherwise determined by the computing device as well understood in the art prior to association with the calendar 5000 for depiction on a specific date. The diagnostic test 100 collected in alternative embodiments may comprise a diagnostic test configured as other than as described herein. In accordance, it is therefore a step of the method of use of the system to engage in allocating the result from the diagnostic test 100, optionally a lateral flow assay test, configured to detect for the presence or absence of at least one additional hormone or hormonal analyte selected from the group consisting of luteinizing hormone, estrogen, E3G, FSH, and human chorionic gonadotropin, to the specific calendar date upon which the result was determined 2006. The present inventor has recognized that such allocation of such a result to such a calendar date may facilitate the subsequent display of such a result within a calendar depicted within the graphical user interface associated with the Patient-Facing Application and/or the Healthcare Professional-Facing Application and further facilitate the identification of trends of hormonal levels which may prove relevant to or otherwise allow for the diagnosis of medical conditions, optionally by a healthcare professional utilizing a Healthcare Professional-Facing Application as described elsewhere herein. Such information may be displayed with and/or in relation to the date of ovulation or date of suspected ovulation as determined in accordance with the mechanisms described elsewhere herein. Resultantly, further steps of the method of use of the system include associating and storing a specific calendar date with the ovulation date of the subject woman 2007 and displaying the results via a graphical user interface 2008 as described herein. In one example, the ovulation date is manually entered via the graphical user interface associated with the Patient-Facing Application, optionally by selecting a date within a calendar depicted therein as described herein. In accordance with the teachings and the components of the system as described elsewhere herein, in various embodiments, the method of use of the system comprises the step of allocating the result from the diagnostic test, optionally a lateral flow assay test configured to detect for at least one additional hormone or hormonal analyte selected from the group consisting of luteinizing hormone, estrogen, an estrogen metabolite such as E3G, FSH, and human chorionic gonadotropin to the specific calendar date upon which the result was determined 2010, optionally in association with the calendar 5000.

In another example, the calendar is depicted on a printed insert within the packaging and designed for utilization in association with a Diagnostic Test Key, the Diagnostic Test Key optionally comprising a color intensity key 800, for visual interpretation of the results of the diagnostic test to allow a lay user of the diagnostic test to manually record results on the calendar in association with the date each of a plurality of diagnostic test results was collected. The calendar depicted on a printed insert is optionally an aspect of the invention in addition to or as a backup to the calendar 5000 configured for depiction within a graphical user interface associated with the Patient-Facing Application.

Scheduler

It is an aspect of various embodiments to provide a Scheduler. The objective of the Scheduler is to establish a telemedicine appointment between a patient user and a healthcare professional user of the system. The Scheduler is configured to operate on a distinct computing device from the computing device operating the Healthcare Professional-Facing Application and also distinct from the computing device operating the Patient-Facing Application, but communicatively connected to both the Healthcare Professional-Facing Application and the Patient-Facing Application, optionally via the internet. The Scheduler is configured to direct the processor of the distinct computing device to access the communicatively connected storage medium of the system to retrieve the periods of availability of all healthcare professional users along with their jurisdictions of licensure and the demographic information of one particular patient user. The Scheduler, via the processor, is configured to then compare the retrieved demographic information of the one particular patient user to the retrieved jurisdictions of licensure of the healthcare professional users.

The Scheduler is configured to aggregate and then display, via the graphical user interface of the Patient-Facing Application operated by the one particular patient user, available periods of availability of healthcare professionals licensed within a jurisdiction matching the patient's demographic information and a graphical user interface element associated with each available period to allow the one particular patient user to select an available period of availability and schedule an appointment, optionally following an additional screen displayed on the user providing an element to allow the one particular patient user to reconfirm his or her intent to schedule an appointment at the indicated time. An example is illustrated by FIG. 12. In one example of the Scheduler, the patient may only retrieve anonymized periods of availability of healthcare professional users without seeing any information pertaining to any individual healthcare professional user, except optionally that the healthcare professional user is licensed to practice in the jurisdiction matching the demographic information of the patient user and/or that the healthcare professional user is capable of treating a condition relevant to the patient user. Optionally, the Scheduler may be configured by the patient user, via the Patient-Facing Application, to display only available periods of availability healthcare professional users capable of treating conditions relevant to the patient. Following the selection of a time by a patient user, the Scheduler is configured to disallow the period of availability for the relevant healthcare professional user from subsequent scheduling by any patient user.

In an example, the Scheduler is configured to activate and display a prompt to activate within the graphical user interface of the Patient-Facing Application following one or more collected results from diagnostic tests indicating a particular condition relevant to the patient user logged in to the Patient-Facing Application. In an example, the Scheduler is configured to automatically activate to aggregate and display the available periods of availability of healthcare professionals licensed within a jurisdiction matching the demographic information of the relevant patient user relevant to a condition indicated by a result of a photographed diagnostic test of the relevant patient user.

The Scheduler, in an example, is further configured to send a notification to a patient user, optionally as a unique message 501 illustrated by FIG. 5, after the patient user activates a graphical user interface element to select an available period of availability and schedule an appointment. The notification to a patient user in various embodiments is provided within the graphical user interface of the Patient-Facing Application, e-mail communication, SMS communication, phone call, or via other communication mechanisms. In various examples, the notification includes a calendar invitation capable of adding the appointment between the patient user and the healthcare professional user relevant to the appointment to a digital calendar formatted as a calendar event, calendar appointment, or calendar invitation, optionally capable for import into a Google Calendar, Apple Calendar or Outlook Calendar managed by the patient user. In an example, the notification to a patient user is made in the form of a calendar appointment that is automatically added to the patient user's calendar by the Scheduler. In various examples, the notification includes a link or instructions to initiate a videoconference meeting between the patient user and the healthcare professional user relevant to the appointment. In various examples, the link or instructions is included within the calendar appointment or calendar invitation.

The Scheduler, in an example, is further configured to send a notification to a healthcare professional user after the patient user activates a graphical user interface element to select an available period of availability directly relevant to the healthcare professional user and schedule an appointment. The notification to a healthcare professional user in various embodiments is provided within the graphical user interface of the Patient-Facing Application, e-mail communication, SMS communication, phone call, or via other communication mechanisms. In an example, the notification to a healthcare professional user is displayed within the electronic health record (EHR) system operated by the healthcare professional user, optionally in addition to the ePHI delivered to the EHR system by the Scheduler in an interoperable format. In such example, the Scheduler may be configured to communicate with an EHR via an application program interface and also may be configured to transmit information in an interoperable format relevant to the communication of digital health information. In various examples, the notification includes a calendar invitation capable of adding the appointment between the patient user and the healthcare professional user relevant to the appointment to a digital calendar formatted as a calendar event, calendar appointment, or calendar invitation, optionally capable for import into a Google Calendar, Apple Calendar or Outlook Calendar managed by the healthcare professional user. In an example, the notification to a healthcare professional user is made in the form of a calendar appointment that is automatically added to the healthcare professional user's calendar by the Scheduler. In various examples, the notification includes a link or instructions to initiate a videoconference meeting between the patient user and the healthcare professional user relevant to the appointment. In various examples, the link or instructions is included within the calendar appointment or calendar invitation.

Following the patient user's selection of available period of availability, the Scheduler is configured to deliver the results of one or more diagnostic test 100 collected via the components described herein and associated with a patient user and available in the communicatively connected storage medium, and/or additional electronic personal health information of the patient user, to the healthcare professional user relevant to the selected period of availability. In various embodiments, the Scheduler is configured to deliver information relevant to a patient into an interoperable format, such as HL7, a clinical document architecture, a continuity of care document or continuity of care record, structured product labeling, clinical context object workgroup, a format relevant to the fast healthcare interoperability resources, a format relevant to the services aware interoperability framework, Arden syntax, formats associated with the Trusted Exchange Framework and Common Agreement and/or other similar interoperable format to allow the interoperable export of information relevant to that patient's profile. In an example, the results of one or more diagnostic tests is directly delivered to the EHR system by the Scheduler, optionally directly via an application program interface (API) and optionally in an interoperable format.

The Patient-Facing Application in an embodiment incorporates a functionality to enable telemedicine via a button element contained within the graphical user interface configured to initiate a video chat with a healthcare provider or a phone call with a healthcare provider in association with the transfer of the relevant information to the healthcare provider.

In various embodiments, the Scheduler is configured to operate via the coding paradigms relevant to the computing devices and processors utilized in association with the system described herein and via methods and mechanisms well understood by those skilled in the art.

Seed Consumption System

The system described herein is configured to provide responsive suggestions for the consumption of certain seeds of a specific type and in a specific amount in an embodiment of the invention. In embodiments of the invention, following the onset of a menstruation, optionally indicated by user entry by the user of the Patient-Facing Application for inclusion into a calendar, a prompt is generated and optionally repeated on a daily basis until the until the first diagnostic test 100 result indicating the presence of luteinizing hormone (LH), to consume, on a daily basis, pumpkin seeds in an amount selected from the range inclusive of 6.5 grams-16 grams and flax seeds in an amount selected from the range inclusive of 6.5 grams-16 grams, or optionally in an amount of 1 tablespoon of pumpkin seeds and 1 tablespoon of flax seeds. The prompt may also optionally include an instruction to pause consumption of sesame seeds and sunflower seeds during the period of consumption of pumpkin seeds and flax seeds, or until the first diagnostic test 100 result indicating the presence of luteinizing hormone (LH) at a threshold to obtain the optimal hormone balancing effect. The prompt may optionally suggest the consumption of a product containing pumpkin and flax seeds in specific amounts, optionally incorporated into a single consumable food item, optionally a snack bar (optionally referred to as a “pumpkin and flax bar.”) The present inventor has discovered that such a combination of pumpkin seeds, in an amount selected from the range inclusive of 6.5 grams-16 grams or an amount of 1 tablespoon, and flax seeds, in an amount selected from the range inclusive of 6.5 grams-16 grams or an amount of 1 tablespoon, into a single consumable food item, optionally a snack bar, and optionally a single consumable food item comprising other ingredients, provides a desirable hormone regulating effect when consumed at specified times in the menstrual cycle, such specified times optionally prompted by the result or results of one or more diagnostic test(s) 100. In an example, the seed consumption system comprises a pumpkin and flax bar in the form of a single consumable food item, optionally a snack bar, consisting of pumpkin seeds, in an amount selected from the range inclusive of 6.5 grams-16 grams or in an amount of 1 tablespoon, crisp sorghum, tapioca syrup, ground flax seeds in an amount selected from the range inclusive of 6.5 grams-16 grams or in an amount of 1 tablespoon, honey and salt.

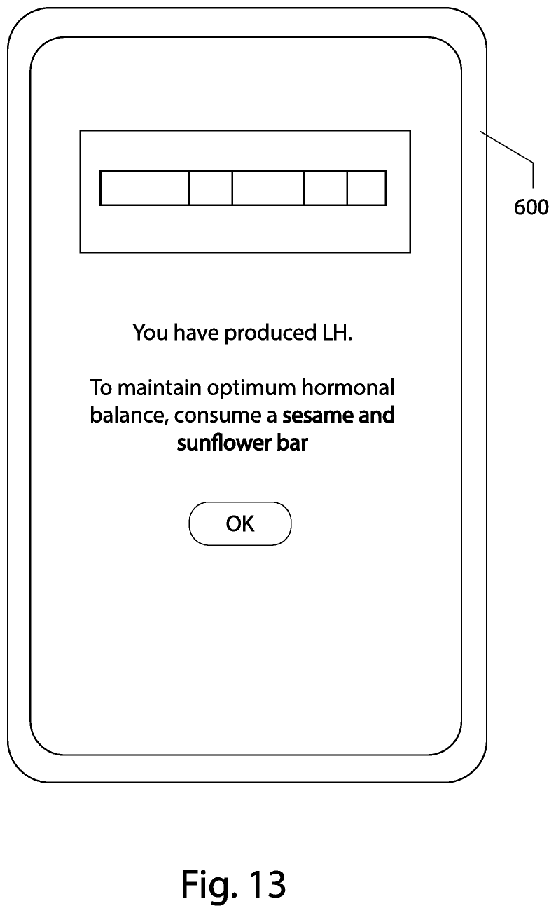

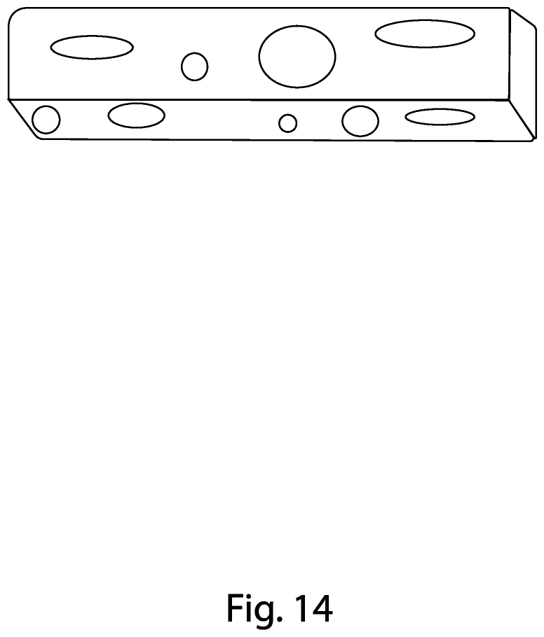

In embodiments of the invention, following the first diagnostic test 100 result indicating the presence of luteinizing hormone (LH), a prompt is generated, and optionally repeated on a daily basis until the onset of menstruation, to consume, on a daily basis, sesame seeds in an amount selected from the range inclusive of 6.5 grams-16 grams or in an amount of 1 tablespoon, and sunflower seeds in an amount selected from the range inclusive of 6.5 grams-16 grams or in an amount of 1 tablespoon, optionally incorporated within a single consumable food item. The prompt may also include an instruction to pause consumption of pumpkin seeds and flax seeds during the period of consumption of sesame seeds and sunflower seeds, and/or until the onset of menstruation, to achieve the optimal hormone regulating effect. The prompt may optionally suggest the consumption of a product containing sesame seeds and sunflower seeds in specific amounts, optionally incorporated within a single consumable food item, optionally a snack bar (referred to as “a sesame and sunflower bar.”) The present inventor has discovered that such a combination of sesame seeds, in an amount selected from the range inclusive of 6.5 grams-16 grams or in an amount of 1 tablespoon, and sunflower seeds in an amount selected from the range inclusive of 6.5 grams-16 grams or in an amount of 1 tablespoon, into a single consumable food item, optionally a snack bar, optionally comprising other ingredients provides the desired hormone regulating effect when consumed at specified times in the menstrual cycle, such specified times optionally prompted by the result or results of one or more diagnostic test(s) 100. An exemplary such prompt is depicted in FIG. 13. In an example, the prompt is displayed within a calendar date of a calendar 5000 in association with the Patient-Facing Application as depicted in FIG. 11. In an example of the invention, the system further comprises a sesame and sunflower bar in the form of a single consumable food item, optionally a snack bar, consisting of crisp sorghum, sunflower kernels in an amount selected from the range inclusive of 6.5 grams-16 grams or in an amount of 1 tablespoon, sesame seeds in an amount selected from the range inclusive of 6.5 grams-16 grams or in an amount of 1 tablespoon, tapioca syrup, honey and salt. An example of the form of a snack bar, representing the general shape and form of either the sesame and sunflower bar or the pumpkin and flax bar in an embodiment, is depicted in FIG. 14.

Various examples of the sesame and sunflower bar and pumpkin and flax bars are packaged together in a single container in quantities corresponding with the optimal intake of a quantity of specified types of seeds to balance hormones in a single menstrual cycle. In various embodiments, a package comprising both a plurality of sesame and sunflower bars and plurality of pumpkin and flax bar comprises quantities of sesame and sunflower bars and pumpkin and flax bars corresponding to that needed for a once daily consumption during either the follicular phase (generally ranging in length from 11 to 27 days) or the luteal phase (generally ranging in length from 11 to 17 days) of the menstrual cycle, optionally with the quantity of a different type of bar relating to the number of days in either the follicular phase or the luteal phase. In various embodiments, the Seed Consumption System comprises such a quantity of sesame and sunflower bars and pumpkin and flax bars included in a single container or package further comprising quantities of diagnostic tests relevant to a single menstrual cycle as described elsewhere herein. In an embodiment, the single container or package further comprises instructions to obtain a Patient-Facing Application, optionally via a printed link on the single container or package or optionally instructions to download the relevant application from the app store via a specific search term. In an embodiment, the Seed Consumption System further comprises the Patient-Facing Application as described elsewhere herein, and further configured to prompt a patient user to consume one or more snack bars comprising sesame and sunflower seeds and/or one or more snack bars comprising pumpkin and flax seeds via a notification generated based on timing and/or the results of a single diagnostic test 100 or a plurality of diagnostic tests that have evaluated a bodily fluid sample collected from the patient user.

In an example, the Patient-Facing Application automatically triggers and facilitates the purchase and delivery of one or more snack bars comprising sesame and sunflower seeds and/or one or more snack bars comprising pumpkin and flax seeds following a single diagnostic test 100 indicating a result for the presence of luteinizing hormone at a threshold in an applied bodily fluid sample. In various embodiments, the Patient-Facing Application is configured to configured to prompt the consumption of the snack bars comprising sesame and sunflower seeds and the snack bars comprising pumpkin seeds and flax seeds at specified times as further described herein. In an alternative configuration, the Patient-Facing Application configured to automatically trigger the purchase and delivery of additional snack bars comprising sesame and sunflower seeds and snack bars comprising pumpkin and flax seeds following the passage of time or following a detected undesirable trend associated with the menstrual cycle as detected by a series of diagnostic tests, detected in association with the Patient-Facing Application as described elsewhere herein. In an example, the purchase and delivery is facilitated, the delivery optionally occurring within two days, via Amazon. In various examples the purchase and delivery takes place on a recurring basis, optionally via a subscription. In various examples the quantity or quantities of sesame and sunflower bars and pumpkin and flax bars, optionally packaged together, purchased correlates to an estimated quantity of bars needed for the once daily consumption of a pumpkin and flax bar during the follicular phase and a sesame and sunflower bar during the luteal phase of a typical menstrual cycle. In varying embodiments, however, and in the present inventor's recognition that menstrual cycles and the associated hormonal levels may vary greatly among healthy women, and especially among women experiencing menopause, perimenopause or puberty, it is an aspect of the invention that the consumption of a specific seed is instructed directly in response to the presence or absence of one or more specified hormone(s) or analyte(s) as indicated by a diagnostic test as described elsewhere herein. In an example, the system features a prescribed action plan offered by a healthcare professional, optionally in association with utilization of the telemedicine system as described elsewhere herein. The prescribed action plan in one example comprises a specified suggested change in diet, optionally comprising components of the seed consumption system such as the sesame and sunflower bars and/or the pumpkin and flax bars, with directions to consume at the specified times as described further herein. In an example, the prescribed action plan offered by a healthcare professional, optionally in association with utilization of the telemedicine system as described elsewhere herein, comprises a specified suggested change in diet guided in association with the utilization of the seed consumption system. Optionally, the method of use of the system comprises a step of communicating a prescribed action plan to the subject woman, the prescribed action plan comprising a specified suggested change in diet 2016, the suggested change in diet optionally comprising consumption of the sesame and sunflower bars and/or the pumpkin and flax bars at the specified times as described further herein.

The present inventor has recognized that the seed consumption system may benefit all women, but will particularly benefit women for which any diagnostic test 100 performed during the period of 7-10 days past ovulation yields at least one result for the absence of PdG at a threshold selected from the range inclusive of 1 μg/mL-10 μg/mL, which could be referred to as a suboptimal cycle. For instance, the present inventor has recognized that the consumption of the seeds as described herein is associated with triggering the increased production of progesterone, correlating to elevated PdG results (as indicated by a plurality of diagnostic tests 100 performed daily each indicating the presence of PdG at a threshold selected from the range inclusive of 1 μg/mL-10 μg/mL during the timeframe of 7-10 days past ovulation, and thereby indicating that a sufficient amount of progesterone has been produced to support conception, for example). The effectiveness of the seed consumption system may be monitored during this time period with additionally performed diagnostic tests 100. For instance, an indication of effectiveness is embodied when a woman, following a suboptimal cycle and her utilization of the seed consumption system in accordance with its teaching, experiences in a subsequent cycle positive results for PdG in association with her use of diagnostic tests on each day during the period of 7-10 days past ovulation. The present inventor has also recognized that in association with diagnostic tests further configured to evaluate for the presence or absence of E3G at a threshold, if such tests indicate a negative result for E3G or the absence of a 1.5 fold increase in E3G during the follicular phase in a subject woman, that the subject woman may have a higher likelihood of benefiting from the consumption of certain seeds in association with her utilization of the seed consumption system as described herein.

Menopause Tracking and Treatment Management System

In an embodiment, the above described system is configured as a menopause tracking system. In various configurations, the menopause tracking system may comprise all or a subset of the Diagnostic Test, Camera, Patient-Facing Application, Healthcare-Facing Application, Storage, Graphical User Interface, Processor, Computing Device, Calendar, and Scheduler as described elsewhere herein.

The preferred embodiment of the Menopause Tracking and Treatment Management system comprises a specially configured Patient-Facing Application as described elsewhere herein. In the special configuration to address the special requirements of the Menopause Tracking and Treatment Management system, the specially configured Patient-Facing Application incorporates features to interpret the results of a diagnostic test 100. In embodiments relevant to the Menopause Tracking and Treatment Management system, the Fertility Tracking System and other embodiments, the diagnostic test is configured as a lateral flow assay featuring three testing zones and corresponding result indication lines, each configured to detect the presence or absence of one of either Lutenizing Hormone (LH), estriol glucuronide (E3G) pregnanediol glucuronide (PdG), each at a pre-defined threshold, in a urine sample applied to the lateral flow assay, in an example as described with more particularity in PCT Patent Application PCT/US18/68027 filed on Dec. 28, 2018 incorporated by reference herein with priority claimed thereto and elsewhere herein. In various embodiments of the invention including those relevant to the Menopause Tracking and Treatment Management System, wherein the diagnostic test 100 comprises one or more thresholds each corresponding to a distinct hormone or hormone analyte, despite comprising the one or more thresholds, the diagnostic test 100 may be performed with other aspects of the invention to provide one or more quantitative result(s), for instance whereby the color intensity of the line can be read be the smartphone reader to determine a hormone or hormone analyte concentration optionally in association with the Diagnostic Test Key 200 or color intensity key 800, such that such indicated result is not merely positive or negative.

In such embodiments, the Patient-Facing Application is configured to utilize the camera of a computing device to photograph the diagnostic test. In such embodiments, the Patient-Facing Application is configured to provide instructions to the processor to determine the indication of a positive or negative result independently for each LH, E3G and PdG tested within a single diagnostic test by measuring the intensity of the color apparent within each result indication line and comparing it to the intensity of the color at the pre-defined threshold, or by another detection mechanism able to detect the result indicated depicted by each result indication line. In an example, in addition to testing zones and corresponding result indication lines configured to detect for the presence or absence of LH, E3G and PdG, the diagnostic test further comprises a testing zone and corresponding result indication line configured to detect FSH. As a persistently high level of FSH may indicate permanent menopause, the detection of FSH in accordance with teachings of the system may trigger a message via the graphical user interface informing the user that she is likely experiencing menopause if persistently high levels of FSH are detected over periods of several consecutive days.

In an example, the menopause tracking and treatment management system further incorporates the seed consumption system and/or the progesterone supplementation system. The present inventors have recognized that prompting the consumption of specified seeds in response to the presence or absence of specific hormones has particular benefit for perimenopausal or menopausal women, namely, the stimulation or suppression of the production of certain hormones in such a manner known to mitigate negative effects of menopause. In an example, the menopause tracking and treatment management system also incorporates mechanisms to suggest a treatment protocol for the supplementation of certain hormones, for example progesterone, in response to certain indications or interpretations of a diagnostic test 100 or a series of diagnostic tests as further described elsewhere herein.

Fertility Tracking System

Embodiments of the invention comprise a fertility tracking system incorporating aspects of the methods described elsewhere herein and the diagnostic test(s) 100 described elsewhere herein. In the preferred embodiment, the system provides information relevant to hormonal levels and changes of hormonal levels over a series of days within a single menstrual cycle, or optionally in comparison to hormonal levels or trends of hormonal levels occurring in previous or subsequent menstrual cycles.

The fertility tracking system in the preferred embodiment comprises a specified quantity of diagnostic tests 100, each diagnostic test 100 consisting of a lateral flow assay, optionally a sandwich assay, each comprising a testing zone and corresponding result indication line configured to detect for the presence or absence of pregnanediol glucuronide in urine at a threshold selected from the range of 1 μg/mL-10 μg/mL. In an embodiment of the diagnostic test 100, the lateral flow assay pertinent to any of PdG and E3G is a competitive assay and the lateral flow assay pertinent to any of LH, FSH and hCG is a sandwich assay. In the preferred embodiment, the lateral flow assay comprising a testing zone and corresponding result indication line configured to detect for the presence or absence of pregnanediol glucuronide is configured to detect for the presence or absence of pregnanediol glucuronide at a threshold of 5 μg/mL. In an embodiment, the diagnostic tests consisting of a lateral flow assay, optionally a sandwich and/or competitive assay, comprising a testing zone and corresponding result indication line configured to detect for the presence or absence of pregnanediol glucuronide in urine are configured such that the presence of only one line (i.e. the control line 105) a positive result and the presence of two lines (i.e. the control line 105 and the first result indication line 107) indicates a negative result for the presence of pregnanediol glucuronide at a threshold selected from the range of 1 μg/mL-10 μg/mL. The present inventor has noted the previous unavailability and unmet need of such diagnostic tests as is well established in the art. In an embodiment, the specified quantity is useful in association with the tracking of hormonal levels on a daily basis over a specified period corresponding to utilization during one single menstrual cycle.

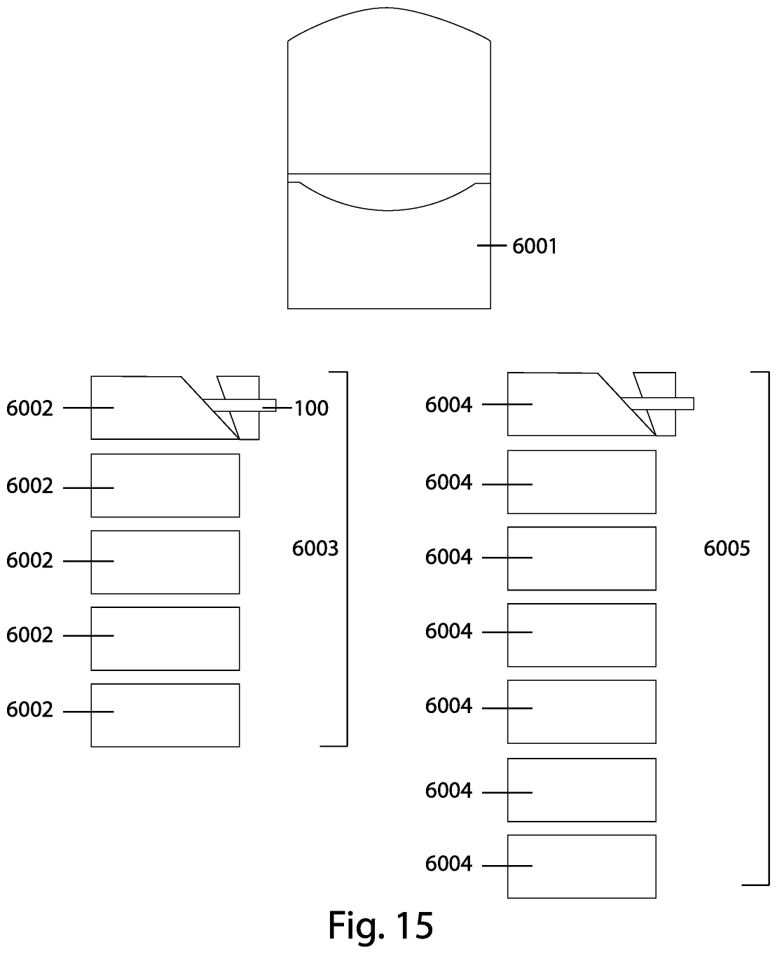

The present inventor has discovered that the quantities of diagnostic tests 100 as described herein is indicated to allow usage of the fertility tracking system by a layperson without the need to calculate the number of diagnostic tests 100 needed based on readings of each diagnostic test 100 or based on the calculation of the length of the menstrual cycle or its phases. In various embodiments, the fertility tracking system comprises a quantity of diagnostic tests, the quantity selected from the range of 4-15 diagnostic tests 6003, each diagnostic test consisting of a lateral flow assay configured to detect for the presence or absence of pregnanediol glucuronide in urine individually placed within a sealed packet 6002. In the preferred embodiment, the fertility tracking system comprises 5 diagnostic tests configured to detect for the presence or absence of pregnanediol glucuronide at a threshold in urine as depicted in FIG. 15. The fertility tracking system further comprises a quantity of diagnostic tests, the quantity selected from the range of 7-25 diagnostic tests 6005, each diagnostic test consisting of a lateral flow assay comprising a testing zone and corresponding result indication line configured to detect for the presence or absence of luteinizing hormone at a threshold chosen from the range of 15 mlU/mL-50 mlU/mL individually placed within a sealed packet 6004 as depicted in FIG. 15. In the preferred embodiment, the lateral flow assay comprising a testing zone and corresponding result indication line configured to detect for the presence or absence of luteinizing hormone is configured to detect for the presence or absence of luteinizing hormone at a threshold of 25 mlU/mL.

The present inventor has recognized that the fertility tracking system comprising the specified quantities of diagnostic tests configured to detect for the presence or absence of pregnanediol glucuronide in urine at a threshold selected from the range of 1 μg/mL-10 μg/mL, and diagnostic tests configured to detect for the presence or absence of luteinizing hormone in urine at a threshold chosen from the range of 15 mlU/mL-50 mlU/mL, aggregated together into a system in accordance with the teachings herein, facilitates usage by a layperson to allow for the collection of data relevant to the functioning of the menstrual cycle on consecutive days without the need for laboratory evaluation of bodily fluids. The specified quantities of diagnostic tests configured to detect for the presence or absence of pregnanediol glucuronide in urine and diagnostic tests configured to detect for the presence or absence of luteinizing hormone in urine, when taken on a daily basis by a layperson user, provides for the collection of a series of test data without the need for a laboratory evaluation. The present inventor has recognized that the quantities of diagnostic tests within the fertility tracking system as described herein correlate to the amounts of diagnostic tests to evaluate the critical fertility hormones relevant to the assessment of one menstrual cycle. In one embodiment, the collection and recording of results on a series of days within the period of a menstrual cycle may take place even without the use of an external device to collect and record results.

In the preferred embodiment, the quantity of diagnostic tests consisting of a lateral flow assay configured to detect for the presence or absence of pregnanediol glucuronide at a threshold in urine and the quantity of diagnostic tests consisting of a lateral flow assay configured to detect for the presence or absence of luteinizing hormone at a threshold in urine are aggregated together into a single package for ease of use by a lay user. The present inventor has noted that the aggregation of such specified quantities of diagnostic tests into a single package solves the need for clarity and simplicity associated with the number of tests needed for a typical single menstrual cycle to test at least for the starting and/or ending dates associated for the highly fertile and highly infertile timeframes of the menstrual cycle. In the preferred embodiment, each diagnostic test associated with the system is packaged into a sealed packet consisting of an airtight foil pouch containing a desiccant package and a single diagnostic test 100. The benefit of the use of an airtight foil pouch is that it maintains the integrity of each diagnostic test 100 well beyond the timeframe associated with a single menstrual cycle.

The technical specifications associated with the novel construction of such diagnostic tests configured to evaluate for the presence or absence of at least pregnanediol glucuronide at a threshold in is disclosed elsewhere herein, and within the following patent applications, with the benefit of priority claimed to each application: U.S. patent application Ser. No. 16/381,229 filed on Apr. 23, 2019; U.S. patent application Ser. No. 16/544,554 filed on Aug. 19, 2019; U.S. Patent Application 62/720,953 filed on Aug. 22, 2018; PCT Patent Application PCT/US18/68027 filed on Dec. 28, 2018; U.S. patent application Ser. No. 16/381,229 filed on Apr. 11, 2019; U.S. patent application Ser. No. 16/732,766 filed on Jan. 2, 2020; and U.S. patent application Ser. No. 16/732,823 filed on Jan. 2, 2020, each of which are incorporated by reference herein.

It could be advantageous in association with the systems and methods disclosed herein to overlap testing associated with LH and PdG. For instance, on the day following the first diagnostic test 100 indicating the presence of LH at a threshold, in the scenario where a separate diagnostic test 100 indicates the absence of LH at a threshold and on the same day a diagnostic test 100 indicates the presence of PdG at a threshold, where the diagnostic test 100 indicating the absence of LH and the diagnostic test 100 indicating the presence of PdG have evaluated the same sample of urine, such result in association with the diagnostic tests indicates that a woman has successfully ovulated in accordance with the teachings of the invention. The present inventor has recognized that such a novel combination of multiple specially configured diagnostic tests (the diagnostic tests specially configured as described elsewhere herein) performed in an intentional fashion over a series of more than one day represents a substantial and important improvement over previously known and available mechanisms for testing.

The present inventor has further recognized that in accordance with the invention, testing for PdG and LH on the same day could be beneficial because if one does not obtain an indication on a diagnostic test indicating the presence of a PdG at a threshold following an indication on a diagnostic test indicating the presence of LH at a threshold on the same day or the previous day, it could mean that a false LH surge has occurred, meaning that the woman has not ovulated yet. A false LH surge refers to a surge in LH hormone that does not result in ovulation or the release of progesterone, correlating to the presence of PdG in urine. Relatedly, in some conditions, such as polycystic ovary syndrome (PCOS), and also in a small percentage of normal menstrual cycles, the surges in LH do not actually result in ovulation. Therefore, in accordance with the present invention the present inventor has discovered that it is advantageous to continue to monitor LH until the subject woman tested has confirmed that ovulation has successfully and/or sufficiently occurred. This is indicated in accordance with a teaching of an embodiment by the presence of serum progesterone which correlates to the presence of pregnanediol glucuronide (PdG) above a threshold in urine. Therefore, as configured, the fertility tracking system as, which combines diagnostic testing for at least both LH and PdG as facilitated by the specially configured diagnostic tests as described elsewhere herein—represents a significant and important improvement over prior art mechanisms and methods featuring only testing for LH. In various embodiments, the aforementioned teachings are utilized in the association of the creation of indications and interpretations, optionally for use in association with a unique message 501, as described elsewhere herein.

The present inventor has recognized the risk of a false LH surge associated with the usage of ovulation predictor kits as known in the prior art, which the teachings of the present invention specifically address and represent a significant improvement over the prior art. Therefore, in an aspect, it is a teaching of the fertility tracking system as described herein to facilitate the simultaneous and/or sequential testing of LH and PdG to mitigate the risk of detecting a false LH surge association with prior art utilization of diagnostic tests configured to detect LH only. The steps of simultaneously and/or sequentially testing for PdG following a positive result for LH forms a teaching of the method embodiment of the invention, and is enabled by the specially configured diagnostic tests 100 configured to evaluate for the presence or absence of PdG at a threshold as described elsewhere herein.

In method embodiment of the invention, it is a teaching to perform the step of evaluating a bodily fluid once daily for the presence or absence of PdG at a threshold during a specified timeframe forming a portion of the menstrual cycle. To facilitate the provision of enough diagnostic tests to accomplish such evaluation without the need for calculation, it is an aspect of the invention to provide a quantity selected from the range inclusive of 4-15 diagnostic tests configured to evaluate for the presence or absence of PdG at threshold. It is an aspect of a method embodiment of the invention that PdG testing should take place daily, for consecutive days up to 10 days past the date of suspected ovulation. In associated steps, the date of the first indicated LH surge in a menstrual cycle is considered the date of suspected ovulation. In an associated method embodiment, the earliest a woman should start testing for PdG in an exemplary method is two days after the date of suspected ovulation. In a method embodiment of the invention featuring the step of evaluating the same bodily fluid sample for both LH and PdG by one or more diagnostic tests configured to evaluate for the presence or absence of both LH and PdG each at a unique threshold, if LH surges on one or more subsequent dates without a corresponding increase in PdG after the one or more previous surges in LH, the date of the most recent subsequent LH surge will replace the all other dates of suspected ovulation, as the new and overriding date of suspected ovulation for that menstrual cycle for purposes of timing the testing of diagnostic tests configured to detect for the presence or absence of pregnanediol glucuronide at a threshold in urine. It is a teaching of a method embodiment to perform the steps of associating and storing a specific calendar date with the ovulation date of the subject woman 2004, whereby the ovulation date is the date of suspected ovulation determined in accordance with the foregoing. It is a teaching of an embodiment that such association and storage of the specific calendar date with the ovulation date takes place in association with the other components of the invention, especially the Processor, the Computing Device, the Calendar, the Graphical User Interface and the Communicatively Connected Storage Medium in an example, each as described elsewhere herein. It is a further teaching of a method embodiment of the invention to perform the step associated with utilization of the system to engage in displaying the results via a graphical user interface 2005 in an example.

As a result, to account for the potential of one or more false LH surges in a single cycle, the present inventor has discovered that it is advantageous to provide a single fertility tracking system comprising a quantity of up to 25 diagnostic tests configured to detect the presence or absence of LH in urine at a threshold within a sealed packet 6005 as described elsewhere herein and additionally a quantity of up to 15 diagnostic tests configured to detect for the presence or absence of pregnanediol glucuronide at a threshold within a sealed packet 6003, optionally a quantity of 8, each such individual diagnostic test 100 configured as described elsewhere herein, aggregated into a single package 6001, optionally to facilitate utilization of the diagnostic tests in association with the methods described herein. In association with intended methods of utilization of the fertility tracking system, the subject woman should perform a diagnostic test 100, the diagnostic test configured to detect for the presence or absence of pregnanediol glucuronide in urine at a threshold selected from the range inclusive of 1 μg/mL-10 μg/mL, to evaluate her urine once daily until 10 days after the date of suspected ovulation. Alternatively or in combination, the subject woman in a related method performs such a diagnostic test on a daily basis during the timeframe inclusive of 7-10 days past the date of suspected ovulation to evaluate her urine, and confirm sufficient ovulation following four consecutive diagnostic tests each taken once daily on four consecutive days each demonstrating an indicated result for the presence of pregnanediol glucuronide in the tested urine, optionally in accordance with the methods disclosed in U.S. patent application Ser. No. 16/732,766, filed on Jan. 2, 2020, hereby incorporated by reference in its entirety herein with priority claimed thereto.

To account for cases where a subject woman evaluating her fertility evidences multiple false LH surges within a single cycle, it is a teaching of a method embodiment to perform the step of evaluating urine for the presence or absence of pregnanediol glucuronide on a daily basis for a plurality of up to 12 consecutive days beyond 2-10 days past suspected ovulation (as indicated by the most recent diagnostic test indicating a positive result for the presence of luteinizing hormone as a threshold), and therefore a fertility testing system comprising a quantity of up to 15 such diagnostic tests (optionally to allow for user errors or associated duplicate testing in an example) or a quantity of as few as 4 such diagnostic tests each configured to detect for the presence or absence of pregnanediol glucuronide in urine is preferred.

On the other hand, for some women (such as those who know that they do not have PCOS, for instance), the risk of a false LH surge is much lower. Therefore, in one example, it is advantageous to have as few as 4 diagnostic tests configured to detect for the presence or absence of pregnanediol glucuronide at a threshold and is a teaching of an embodiment. Such a quantity corresponds to that necessary to evaluate urine on a once daily basis between the dates inclusive of 7-10 days after the date of suspected ovulation, in accordance with methods described with more particularity in U.S. patent application Ser. No. 16/732,766 filed on Jan. 20, 2020, which as noted previously this application claims the benefit of and incorporates by reference in its entirety. For example, a sequence of diagnostic tests taken daily on the dates including days 7-10 past the suspected ovulation date is suggestive of proper corpus luteum functionality in one intended method of usage of the fertility tracking system.

For similar reasons, it is likewise advantageous in an example to incorporate as few as 7 diagnostic tests configured to detect for the presence or absence of luteinizing hormone at a threshold in urine into the fertility tracking system. For cost saving or efficiency purposes, such a configuration may be preferred in the cases of healthy women with healthy cycles. The quantity of tests necessary for a single fertility testing system intended for use during a single menstrual cycle is informed by the needs of a healthy woman in one example. In such an instance, an example result may occur where LH testing is performed on a daily basis starting on the 10^{th} day of the menstrual cycle, indicating a LH surge on the third day of testing for LH (the 13^{th} day of the menstrual cycle), with the testing for PdG commencing on the second day following the first test indicating the presence of LH at a threshold in urine (the 15^{th} day of the menstrual cycle) in accordance with the preferred method of use of the fertility tracking system, that test indicating the presence of PdG in urine at a threshold on the 15^{th} day of the menstrual cycle. In such a case only five diagnostic tests configured to detect for the presence or absence of LH at a threshold would need to be performed. Further, in accordance with the preferred method of use, only four diagnostic tests configured to detect for the presence or absence of PdG would need to be performed in a single menstrual cycle, each on a daily basis during the period of 7-10 days past suspected ovulation. It is a teaching of an alternative embodiment that once an LH test results in a positive reading correlating to an LH surge, generally (though not always) following a negative LH test, this signifies the time to change to commence testing for PdG over a timeframe potentially preceding the 7-10 days past suspected ovulation window. In various embodiments, the systems associated with the invention are configured to incorporate such quantities.

The present inventor has recognized that the quantity of diagnostic tests configured to detect for the presence or absence of LH at a threshold is optimally selected from the range inclusive of 7-25 such diagnostic tests, in part because a subject woman may benefit from testing for LH on multiple times per day in certain circumstances. For example, the present inventor recognizes that LH can surge in the afternoon or the morning, and the surge could be for a very short duration. Therefore the present inventor has further recognized that in order to detect the transient increase in LH, testing multiple times per day can be advantageous. Therefore, as LH may be more advantageous in the afternoon as opposed to the morning, or in addition to testing in the morning, a teaching of which is incorporated into an embodiment of the invention, such teaching represents a significant departure from the teachings associated with LH testing in the prior art, which recommend protocols associated with only testing in the morning. Such teachings relate to the quantity of diagnostic tests configured to detect for the presence or absence of LH to incorporate into the system in association with additional diagnostic tests at least comprising a plurality of diagnostic tests configured to detect for the presence or absence of PdG chosen in association with embodiments of the system described herein.

In an embodiment of the invention, the intended use of testing for LH and the testing for PdG occurs via a single strip containing separate result indication lines each configured to evaluate for the presence or absence of a distinct hormone or analyte each at a threshold. Therefore, in association with such intended use, the system—instead of comprising two categories of diagnostic tests, each category of diagnostic test comprising a quantities of diagnostic tests configured to evaluate for the presence or absence of only one hormone or analyte—alternatively comprises as few as 10 and as many as 25 diagnostic tests configured to evaluate for at least for the presence or absence of PdG at a threshold in a first testing zone corresponding to a first result indication line 107 and the presence or absence of LH at a threshold in a second testing zone corresponding to a second result indication line 108. In an example, each diagnostic test 100 comprises a single lateral flow assay comprising a plurality of separate testing zones, each testing zone configured to evaluate a separate hormone or analyte as described elsewhere herein. As such it is a teaching of an embodiment of the invention to comprise a single package 6001, optionally a cardboard box, comprising a quantity of diagnostic tests selected from the range inclusive of 10-25 diagnostic tests, wherein each diagnostic test comprises a single lateral flow assay comprising a plurality of separate testing zones, each testing zone configured to evaluate for the presence or absence of a separate hormone or analyte at a threshold and to present a visual result on a corresponding result indication line.

In addition, whether or not incorporated into a single diagnostic test configured to evaluate urine for the presence or absence of a plurality of hormones and/or analytes each at a unique threshold as described elsewhere herein, an example of the invention additionally comprises diagnostic tests comprising testing zones and corresponding result indication lines other than those configured to detect for the presence of LH and PdG. In an example of the invention, diagnostic tests configured to evaluate urine for the presence or absence of FSH at a threshold are included at a quantity selected from the range inclusive of 7-25 diagnostic tests. The present inventor has recognized that a diagnostic test configured to evaluate for the presence or absence of FSH at a threshold is useful, as the presence of FSH signifies when the fertile window opens in the menstrual cycle, as a follicle is “stimulated” or selected by follicle stimulating hormone (FSH), thereby opening the fertile window. The presence of FSH, specifically the presence of FSH in an amount equivalent to a 1.5 fold decrease as compared to a prior diagnostic test indicating the presence of FSH performed in the same menstrual cycle, such fold change optionally determined by comparing the indicated colors to the colors indicated on the color intensity key 800 and associating the indicated FSH concentration with each diagnostic test 100, signifies the stimulation of the follicle and also confirms the opening of the fertile window. For instance, FSH is generally elevated in the beginning of a healthy menstrual cycle. Measuring FSH is also uniquely beneficial for women experiencing menopause or perimenopause in association with the menopause tracking and treatment management system described elsewhere herein, as a persistently high level of FSH may indicate permanent menopause. In an example, the indication for FSH in association with various embodiments may provide a marker for ovarian reserve. In various embodiments, the results indicated by diagnostic test 100 configured to evaluate for the presence of FSH are used in association with other elements of the system to generate indications and/or interpretations as described elsewhere herein, each optionally for display as a unique message 501.

In an example, the invention further comprises diagnostic tests configured to evaluate urine for the presence of Estrogen Metabolite (E3G), included at a quantity selected from the range inclusive of 7-25 diagnostic tests. The presence of E3G, specifically the presence of E3G in an amount equivalent to a 1.5 increase as compared to a prior diagnostic test 100 indicating the presence of E3G taken in the same menstrual cycle, such fold change optionally determined by comparing the indicated colors to the colors indicated on the color intensity key 800 and associating the indicated E3G concentration with each diagnostic test 100, signifies the maturity of the follicle and also confirms the opening of the fertile window. Examples of the invention incorporating a plurality of diagnostic tests configured to detect for the presence or absence of E3G can provide additional information. For instance, the present inventor has recognized that in comparison to a diagnostic test indicating the presence FSH, a diagnostic test configured to evaluate for the presence of E3G provides a slightly different and potentially complementary indication, namely that after the follicle was selected (signaled by FSH) the maturing follicle has secreted estrogen.

In an exemplary sequence of testing featuring a plurality of diagnostic tests 100 each configured to evaluate only one hormone or analyte, a method embodiment comprises the step of instructing the subject woman, optionally via the graphical user interface, to commence utilizing a diagnostic test configured to detect the presence or absence of FSH in an applied fluid once daily 9001, optionally at a threshold or optionally by comparison of the degree of change compared to an earlier diagnostic test configured to detect the presence or absence of FSH as described elsewhere herein, on a daily basis commencing on a day chosen from the range inclusive of 2-3 days following the onset of a subject woman's menstruation in a menstrual cycle. In an exemplary method, the step of collecting, on a daily basis, the results of each of a plurality of diagnostic tests by comparing the color intensity indicated on each diagnostic test to a color intensity key 800 to estimate the FSH concentration 9002. Such an exemplary sequence is depicted by FIG. 16.

Following a change in FSH as indicated by a diagnostic test subsequent to at least one earlier taken diagnostic test indicating the presence of FSH in the same menstrual cycle—the change indication optionally representing a 1.5 fold decrease in FSH levels, and optionally detected in association with use of the Patient Facing App, Processor, Computing Device and/or Camera as described elsewhere herein—an exemplary method further comprises the step of instructing the subject woman, optionally via the graphical user interface, to commence utilizing a diagnostic test comprising a testing zone and corresponding result indication line configured to detect the presence of E3G once daily on a daily basis 9003. In an alternative embodiment, the change is indicated or for example by a result on a diagnostic test configured to detect FSH in urine at a threshold demonstrating a level indicated above a threshold followed by a result on a diagnostic test configured to detect FSH in urine demonstrating an indicated level of FSH below a threshold, In an example, the change in FSH is determined by photographing a diagnostic test comprising at least one testing zone configured to evaluate the presence of FSH in the bodily fluid to determine a baseline indicated color intensity in a corresponding result indication line on a date 2-3 days from the onset of menstruation. In an embodiment, a Patient-Facing Application or digital device is preconfigured with color intensities of such result indication line corresponding to different levels of FSH in an applied fluid, for instance by recording the results for the color intensities occurring in each of a plurality of diagnostic tests each applied with a spiked male urine including known amounts of FSH, optionally in association with the color intensity key 800. In an example, the applied fluid consists of male urine spiked with a known level of FSH. In various embodiments where the color intensity associated with results of a diagnostic test does not precisely match a previously known correlation to an amount of a hormone or analyte (such as FSH or E3G) in urine, the estimated amount is instead determined by substituting the closest color intensity to the indicated color intensity and estimating the level of the amount of hormone or analyte in the applied fluid to be that associated with the closest color intensity. In such manner, the present inventor has recognized that it is possible to determine fold changes in FSH in an applied fluid, for example a 1.5 fold decrease, evidenced by one diagnostic test as compared to a previously taken diagnostic test as a baseline, by correlating color intensities indicated on a diagnostic test to a known level of FSH and ensuring that in methods of use that each diagnostic test utilized in association with the relevant method is similarly configured. Similar mechanisms are useful in correlating color intensities with the levels in an applied bodily fluid of other hormones and/or analytes, such as E3G. Such correlated color intensities are useful in an embodiment comprising a Diagnostic Test Key 200 as described elsewhere herein. It is important to note in association with teachings of the invention that a certain fold decrease in the color intensity indicated over a series of diagnostic tests does not necessarily equate to a similar fold decrease or increase in the actual relevant amount of hormone or analyte in an applied sample. Notably, the present inventor has determined that some diagnostic tests configured with certain carrier proteins are difficult to consistently reproduce, and has also noted that the precision associated with manufacturing consistently reproduced diagnostic tests is important in the effective deployment of such method.

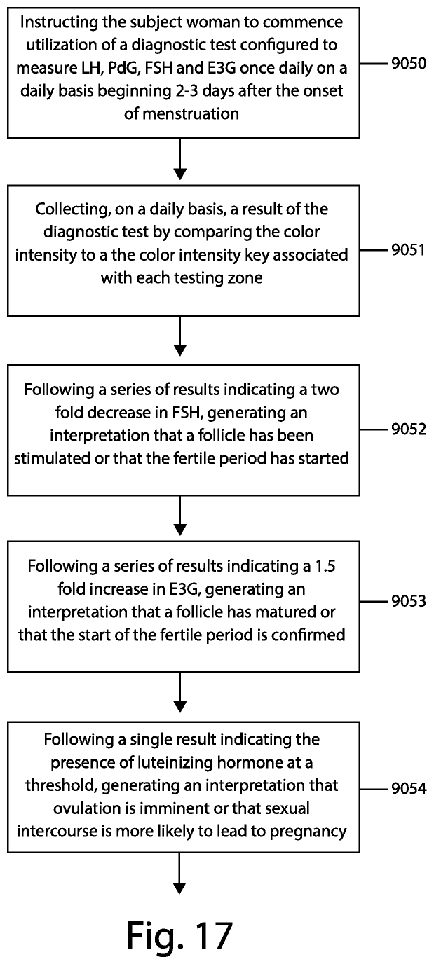

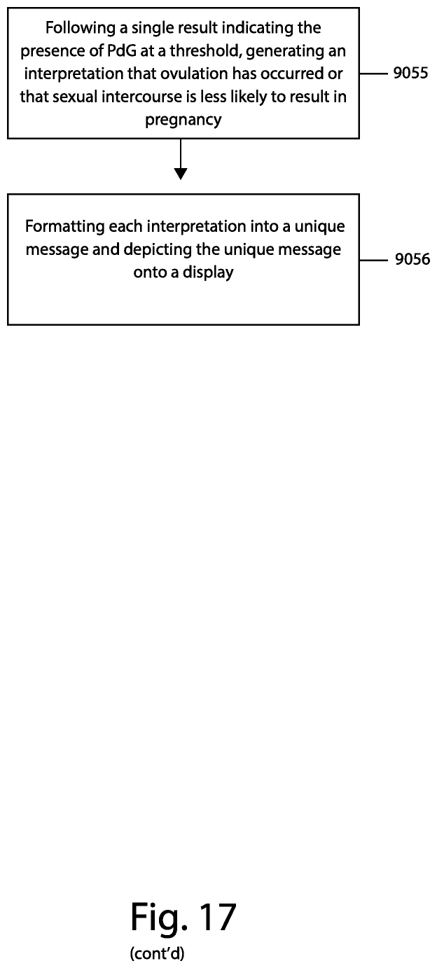

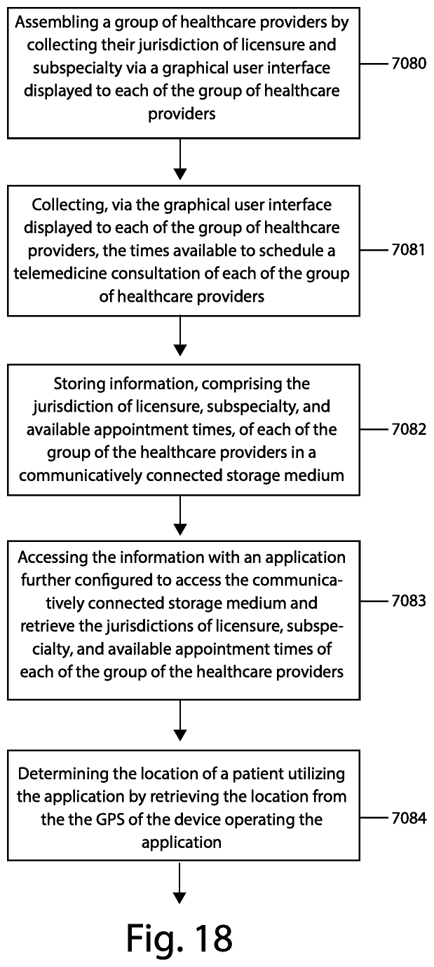

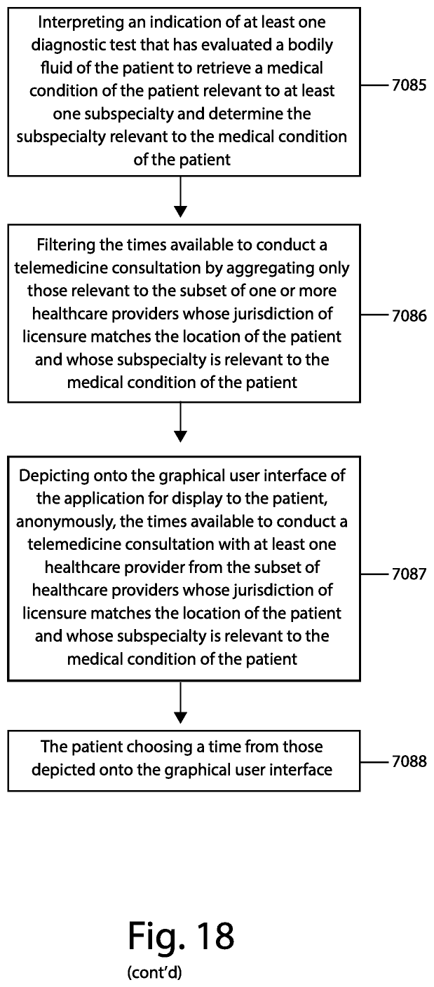

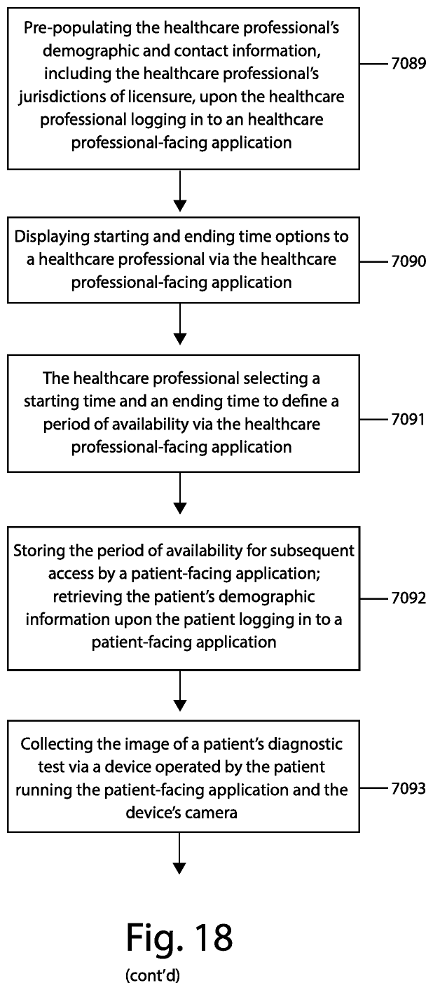

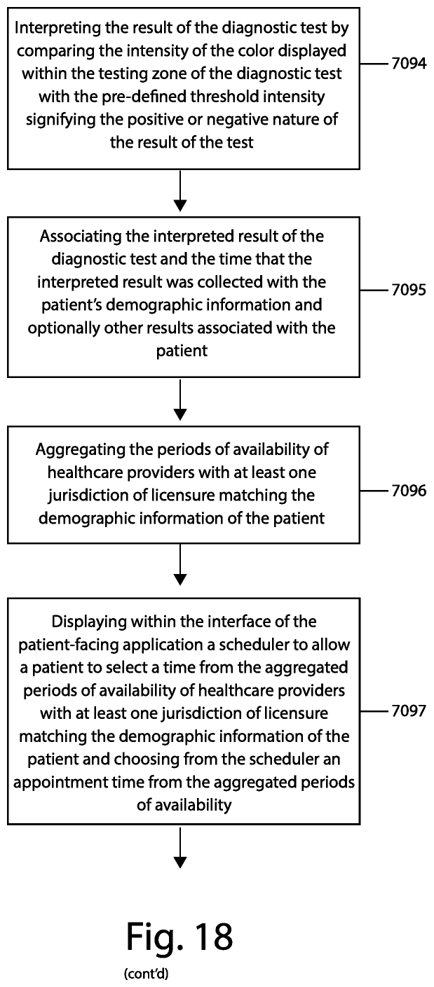

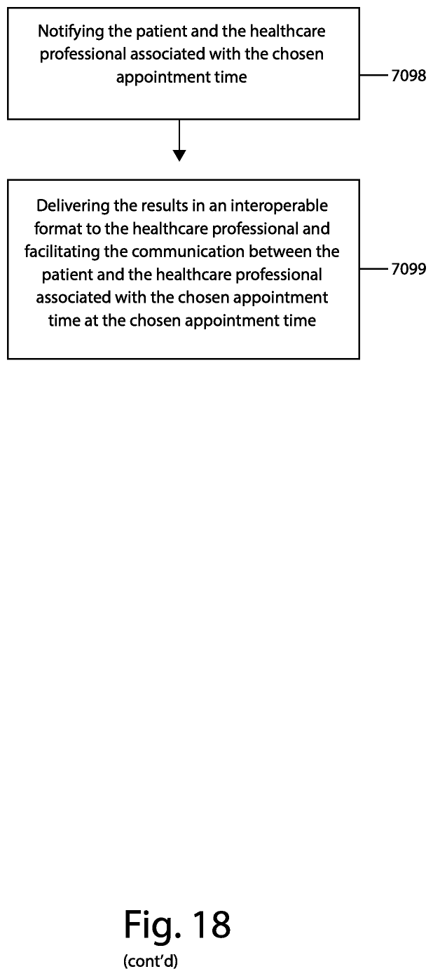

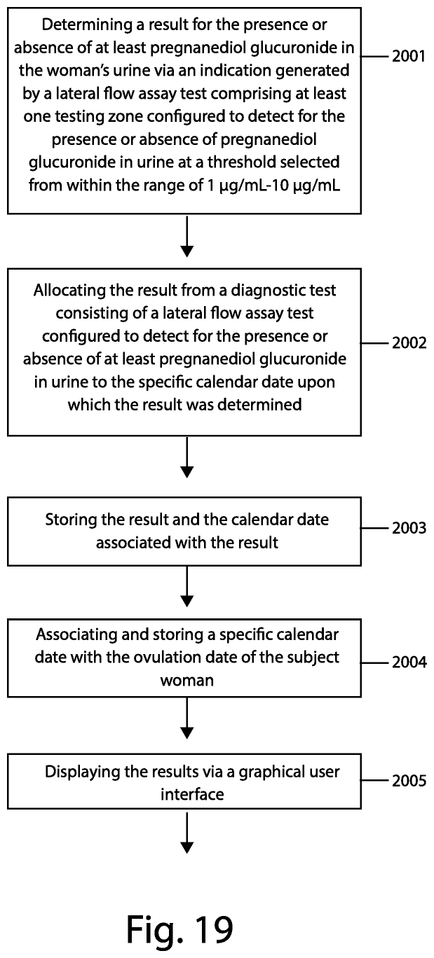

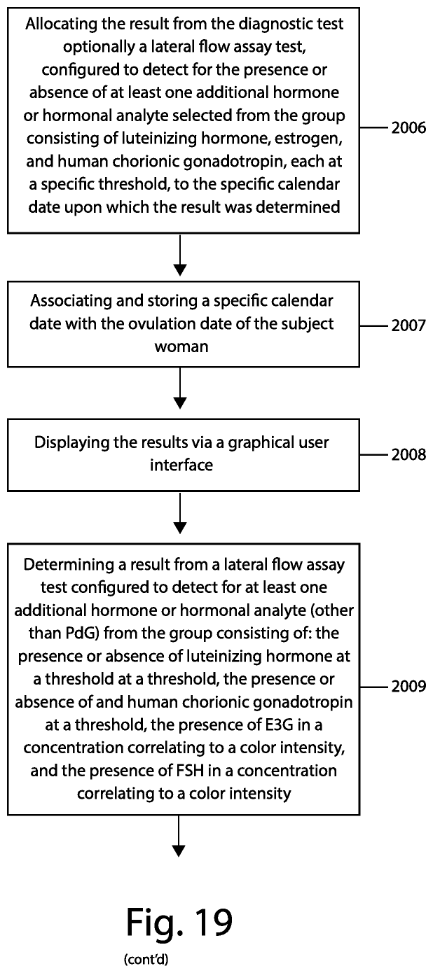

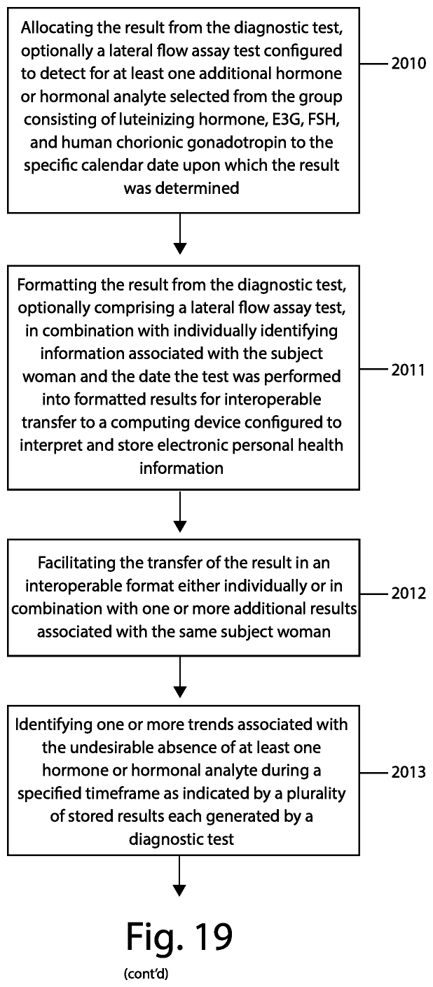

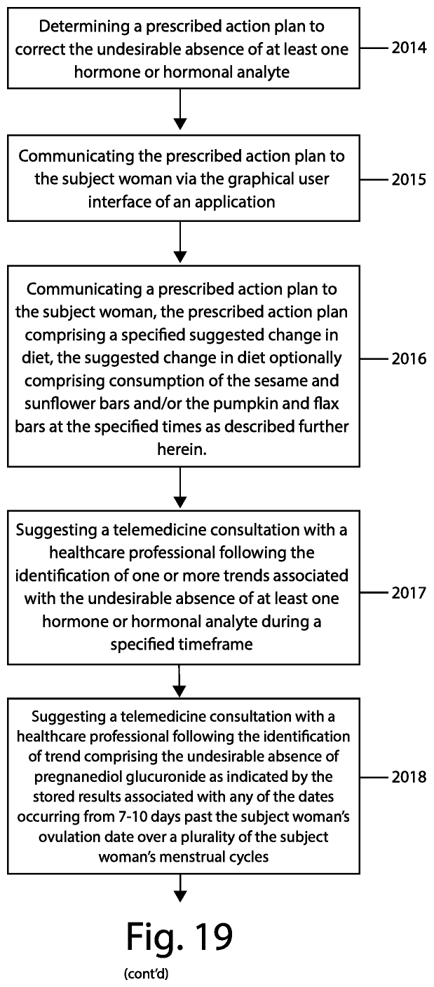

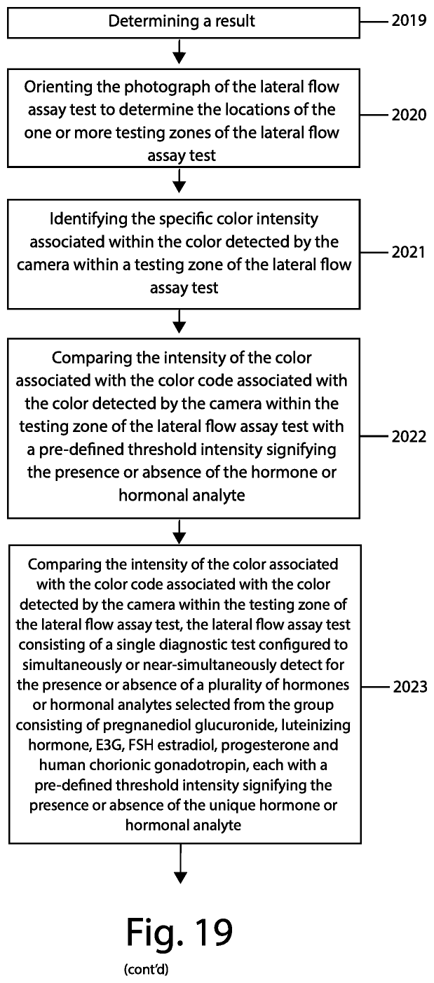

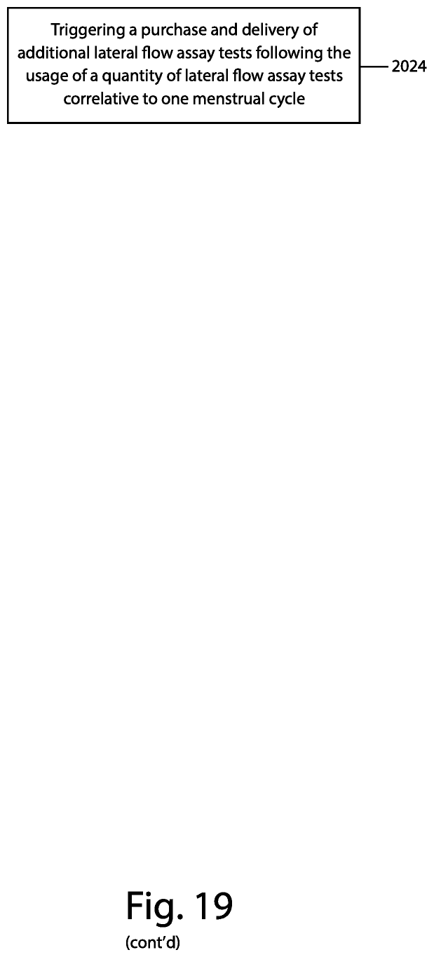

Following a detected change in FSH indicated as described herein, a user is instructed, optionally via the graphical user interface, to commence utilizing a diagnostic test comprising a testing zone and corresponding result indication line configured to detect the presence of E3G once daily on a daily basis 9003. The instruction, optionally configured as a unique message 501 as described elsewhere herein, optionally is delivered to the user of a Patient-Facing Application via the graphical user interface following the positive result for the presence of FSH in urine that the follicle has stimulated and/or that the fertile window has opened. In an embodiment of the invention utilizing a diagnostic test 100 comprising a plurality of testing zones and corresponding result indication lines comprising one testing zone and corresponding result indication line configured to detect for the presence or absence of E3G at a threshold and further comprising another testing zone and corresponding result indication line configured to detect for the presence or absence of FSH at a threshold, an instruction to change the type of diagnostic test 100 utilized is not necessary and therefore not included. (Instead, the steps associated with instructing a subject woman to commence utilization of a diagnostic test on a daily basis 9050 and collecting, on a daily basis the result of each diagnostic test performed 9051, as further described in FIG. 17 are repeatedly performed as an alternative). In such alternative, the preferred method embodiment incorporates a step of repeating use of the diagnostic test 100 comprising a plurality of testing zones and corresponding result indication lines, each of which is configured to detect for the presence or absence of a distinct hormone or analyte, once daily on a daily basis throughout the period of fertility testing, as switching between a different type of tests each separately configured to detect for a different subset of hormones and/or analytes becomes unnecessary in such example. In various method embodiments, following the first indication demonstrating the presence of FSH at a threshold on a diagnostic test 100, a unique message 501 is delivered to the user of the system via the graphical user interface that the follicle has stimulated and/or that the fertile period of the subject woman's menstrual cycle has begun.

In an exemplary implementation, it is a further teaching to perform the step of recording and evaluating the results of the testing for FSH collected from a diagnostic test 100 performed daily, and any results from any subsequently performed diagnostic tests configured to evaluate additional hormones and/or analytes in an applied bodily fluid, optionally in association with other components of the system as described herein. As depicted on FIG. 17 and elsewhere herein, the method further comprises the step of generating an interpretation following the evaluation of a result of each diagnostic test 100, the interpretation based on the specific indicated result 9051-9055. Following each of the generating an interpretation steps 9051-9055, the method further comprises the step of formatting each interpretation into a unique message 501 and depicting the unique message 501 onto a display 605.

Following a change in FSH as indicated by a diagnostic test 100 subsequent to at least one previously positive diagnostic test for FSH in the same menstrual cycle—optionally a 1.5 fold decrease in FSH levels, and optionally detected by the Patient Facing App, processor, computing device and camera as described elsewhere herein—a user is instructed, optionally via the graphical user interface, to commence utilizing a diagnostic test 100 comprising a testing zone and corresponding result indication line configured to detect and provide an indication correlating to a color intensity for the presence of E3G once daily on a daily basis 9003, the color intensity optionally indicating an amount of E3G in association with a color intensity key 800 and/or a Diagnostic Test Key 200. In an alternative embodiment, the change is indicated by a result on a diagnostic test 100 configured to detect FSH in urine demonstrating a level indicated above a threshold followed by a result on a diagnostic test configured to detect FSH in urine demonstrating an indicated level of FSH below a threshold. In an example, the change in FSH is determined by photographing a diagnostic test 100 comprising at least one testing zone and a corresponding result indication line configured to evaluate for the presence of FSH in the bodily fluid to determine a baseline indicated color intensity, optionally corresponding to a specific RGB or HEX color code, in such testing zone on a date 2-3 days from the onset of menstruation. In an embodiment, a Patient-Facing Application or digital device is preconfigured with color intensities, optionally derived from a color intensity key 800, of such result indication line corresponding to different levels of FSH in an applied fluid, for instance by recording the results for the color intensities occurring in each of a plurality of diagnostic tests each applied with fluid containing a known amount of FSH. In an example, the applied fluid consists of male urine spiked with a known level of FSH. In various embodiments where the color intensity associated with results of a diagnostic test 100 does not precisely match a previously known correlation to an amount of a hormone or analyte (such as FSH or E3G) in urine, the estimated amount is instead determined by substituting the closest color intensity included in the color intensity key 800 to the indicated color intensity and estimating the level of the amount of hormone or analyte in the applied fluid to be that associated with the closest color intensity. In such manner, the present inventor has recognized that it is possible to determine fold changes in FSH in an applied fluid, for example a twofold decrease, evidenced by one diagnostic test as compared to a previously taken diagnostic test as a baseline, by correlating color intensities indicated on a diagnostic test to a known level of FSH and ensuring that in methods of use that each diagnostic test utilized in association with the relevant method is similarly configured. Similar mechanisms are useful in correlating color intensities with the levels in an applied bodily fluid of other hormones and/or analytes, such as E3G. Such correlated color intensities are useful in an embodiment comprising a Diagnostic Test Key 200 as described elsewhere herein. It is important to note in association with teachings of the invention that a certain fold decrease in the color intensity indicated over a series of diagnostic tests does not necessarily equate to a similar fold decrease or increase in the actual relevant amount of hormone or analyte in an applied sample. Notably, the present inventor has determined that some diagnostic tests configured with certain carrier proteins are difficult to consistently reproduce, and has also noted that the precision associated with manufacturing consistently reproduced diagnostic tests is important in the effective deployment of such method.

In an embodiment of the invention utilizing a diagnostic test comprising a plurality of testing zones comprising one testing zone configured to detect for the presence or absence of E3G at a threshold and further comprising another testing zone configured to detect for the presence or absence of FSH at a threshold, an instruction to change the type of diagnostic test utilized is not necessary and therefore not provided, as in the method depicted by FIG. 17. In such alternative, it is advantageous instead to instruct the user to simply repeat use of an identically configured diagnostic test 100 comprising a plurality of testing zones, each of which is configured to detect for the presence or absence of a distinct hormone or analyte, once daily on a daily basis throughout the period of fertility testing. In an embodiment, following the first indication demonstrating the presence of FSH at a threshold on a diagnostic test, a message is delivered to the user of the system via the graphical user interface that the follicle has stimulated and/or that the fertile window has opened.

In various embodiments the length of the menstrual cycle is determined as the duration between the onset of menstruation in a first menstrual cycle and the onset of menstruation in the subsequent menstrual cycle. An average menstrual cycle length can be calculated by looking back at a number of recent menstrual cycles, optionally 6 menstrual cycles, and determining the average in accordance with basic mathematical principles. In various embodiments, the average length may be entered via the Patient-Facing Application or a digital device to assist in the determination of the dates for when testing for various hormones should change. It is a teaching of a method embodiment that to determine the day upon which testing for FSH should be replaced or augmented with testing for E3G, a Patient-Facing Application could be configured to subtract 21 from the average length of a subject woman's menstrual cycle to arrive at an approximated length of time from the onset of menstruation in a single menstrual cycle until the detectible presence of E3G in tested urine, which could be used as a backup timeframe to change use of diagnostic tests configured to detect only one particular hormone or analyte on a daily repeating basis, if a 1.5 fold decrease in FSH is not detected over a series of diagnostic tests within such an approximated timeframe. In an embodiment, in a case where a 1.5 fold decrease in FSH is not observed on any of a series of diagnostic tests performed on a subject woman's bodily fluid within a single menstrual cycle, a message is delivered via the graphical user interface or otherwise to a display stating that ovulation is likely not to occur this cycle. In an embodiment, in a case where a 1.5 fold decrease in FSH is not observed on any of a series of diagnostic tests performed on a subject woman's bodily fluid within a single menstrual cycle, optionally only if the woman is above a certain age (in an example, 35 years old) a message is delivered via the graphical user interface or otherwise to a display suggesting a likelihood that menopause has started. In an embodiment, whereby the system and/or the user has detected three or more consecutive non-ovulatory cycles and the user is below a certain age (in an example, 35 years old), a message is delivered via the graphical user interface or otherwise to a display suggesting a likelihood that the woman is experiencing PCOS or another medical condition and that it would be appropriate to discuss with a physician. In an embodiment, whereby the system and/or the user has three or more consecutive non-ovulatory cycles and the user is above a certain age (in an example, 35 years old), a message is delivered via the graphical user interface or otherwise to a display suggesting a likelihood that menopause has started. In an embodiment, where a 1.5 fold decrease in FSH is observed on any diagnostic test within a series of diagnostic tests performed on a subject woman's bodily fluid within a single menstrual cycle, a message is delivered via the graphical user interface or otherwise to a display suggesting that a follicle has been selected and/or that the subject woman's fertile window has opened. The present inventor has recognized that such information is useful to a subject woman in association with maximizing her chances for conception during a specific menstrual cycle.

In accordance with such sequence, which comprises a method embodiment of the invention, and its related teachings as described herein, a first step comprises testing for FSH, commencing on a date selected from the range inclusive of 2-3 days following the onset of menstruation in a subject woman's menstrual cycle, by evaluating a bodily fluid of the subject woman by utilizing a first lateral flow assay comprising at least a testing zone and corresponding color intensity line configured to evaluate the presence of FSH in the bodily fluid to determine a baseline indicated color intensity in the result indication line corresponding to a level of FSH in the bodily fluid 3001.

Further in accordance with such sequence and teachings, a second step comprises repeating testing of the subject woman's bodily fluid for FSH once daily with a series of subsequently performed lateral flow assays each comprising at least a testing zone configured to evaluate the presence of FSH and corresponding result indication line, until the color intensity evident on the result indication line of one of the lateral flow assays performed within the series corresponds to a color intensity evidencing at least a 1.5-fold decrease in the level of FSH in the bodily fluid as compared to the level of FSH corresponding to the baseline indicated color intensity evident in the result indication line configured to provide an indication for the presence of FSH in the bodily fluid of the first lateral flow assay 3002.

Further in accordance with such sequence and teachings, a third step comprises following the earlier of an evidenced at least 1.5-fold decrease in the presence of FSH within a series of previously taken diagnostic tests in the same menstrual cycle or the passage of 8 days following from the onset of menstruation of the same menstrual cycle of the subject woman, commencing testing for E3G by performing a lateral flow assay comprising at least a testing zone and corresponding result indication line configured to evaluate the presence of E3G in a bodily fluid to determine a baseline indicated color intensity for E3G in the result indication line corresponding to a level of E3G in the bodily fluid 3003. In an example, following a change in FSH as indicated by a diagnostic test subsequent to at least one a previously positive diagnostic test 100 for FSH in the same menstrual cycle—optionally a 1.5 fold decrease in FSH levels, and optionally detected by the Patient Facing App, digital reader 670, processor, computing device and camera as described elsewhere herein—a user is instructed, optionally via the graphical user interface, to commence utilizing a diagnostic test 100 comprising a testing zone and corresponding result indication line configured to detect the presence of E3G once daily on a daily basis. In an alternative embodiment, the change is indicated by a result on a diagnostic test 100 configured to detect FSH in urine at a threshold demonstrating a level indicated above a threshold followed by a result on a diagnostic test configured to detect FSH in urine demonstrating an indicated level of FSH below a threshold. In an example, the change in FSH is determined by photographing a diagnostic test 100 comprising at least one testing zone and corresponding result indication line configured to evaluate the presence of FSH in the bodily fluid to determine a baseline indicated color intensity in such result indication line on a date 2-3 days from the onset of menstruation. In an embodiment, a Patient-Facing Application or digital reader 670 is preconfigured with color intensities of such result indication line corresponding to different levels of FSH in an applied fluid, for instance by recording the results for the color intensities occurring in each of a plurality of diagnostic tests 100 each applied with a spiked male urine including known amounts of FSH. In an example, the applied fluid consists of male urine spiked with a known level of FSH. In various embodiments where the color intensity associated with results of a diagnostic test does not precisely match a previously known correlation to an amount of a hormone or analyte (such as FSH or E3G) in urine, the estimated amount is instead determined by substituting the closest color intensity to the indicated color intensity and estimating the level of the amount of hormone or analyte in the applied fluid to be that associated with the closest color intensity. In such manner, the present inventor has recognized that it is possible to determine fold changes in FSH in an applied fluid, for example a 1.5 fold decrease, evidenced by one diagnostic test as compared to a previously taken diagnostic test as a baseline, by correlating color intensities indicated on a diagnostic test to a known level of FSH and ensuring that in methods of use that each diagnostic test utilized in association with the relevant method is similarly configured. Similar mechanisms are useful in correlating color intensities with the levels in an applied bodily fluid of other hormones and/or analytes, such as E3G. Such correlated color intensities are useful in an embodiment comprising a Diagnostic Test Key 200 as described elsewhere herein. It is important to note in association with teachings of the invention that a certain fold decrease in the color intensity indicated over a series of diagnostic tests does not necessarily equate to a similar fold decrease or increase in the actual relevant amount of hormone or analyte in an applied sample. Notably, the present inventor has determined that some diagnostic tests configured with certain carrier proteins are difficult to consistently reproduce, and has also noted that the precision associated with manufacturing consistently reproduced diagnostic tests is important in the effective deployment of such method. In an example. a user is instructed, optionally via the graphical user interface, to commence utilizing a diagnostic test comprising a testing zone and corresponding result indication line configured to detect the presence of E3G once daily on a daily basis. The message optionally is delivered to the user of the system via the graphical user interface following the positive result for the presence of FSH in an applied bodily fluid of at least a specified amount that the follicle has stimulated and/or that the fertile window has opened. In an alternative embodiment, such as an embodiment configured to evaluate a fluid other than urine, as an alternative to testing for E3G it may be advantageous to test the fluid (i.e. saliva or blood) for estrogen.

Further in accordance with such sequence and teachings, a subsequent step comprises repeating the testing for E3G once daily with a series of subsequently performed lateral flow assays each comprising at least a testing zone and corresponding result indication line configured to evaluate the presence of E3G in a bodily fluid, until the color intensity evident within the result indication line configured to evaluate for the presence of E3G in a single lateral flow assay within the series displays a color intensity evidencing at least a 1.5-fold increase in the level of E3G in the tested bodily fluid as compared to the level of E3G corresponding to the baseline indicated color intensity for E3G 3004.

On the day following a result on a subsequently taken diagnostic test indicating that E3G has increased in an amount of at least 150% (also defined as a 1.5 fold increase, optionally detected via similar mechanisms associated with detecting a fold change of indicated FSH as described elsewhere herein) from the first diagnostic test taken after the twofold decrease in FSH (or in the case where FSH did not evidence a 1.5 fold decrease in the same menstrual cycle, the passage of 10 days following the date of the onset of menstruation in the same menstrual cycle of the subject woman), LH testing should commence in accordance with teachings of an embodiment and be repeated on a daily basis until a result on a diagnostic test comprising a testing zone and corresponding result indication line configured to detect for the presence or absence of LH at a threshold indicates the presence of LH at a threshold, in accordance with teachings of an embodiment. In an example, the method embodiment further comprises providing an instruction to a user to commence utilization of a diagnostic test 100 configured to detect for the presence of LH at a threshold once daily on a daily basis. In an embodiment of the invention utilizing a diagnostic test 100 comprising a plurality of testing zones comprising one testing zone configured to detect for the presence or absence of E3G at a threshold and further comprising another testing zone configured to detect for the presence or absence of LH at a threshold, such instruction is not necessary and therefore not included. In such alternative, it is advantageous instead to instruct the user to simply repeat use of the diagnostic test 100 comprising a plurality of testing zones and corresponding result indication lines, each of which is configured to detect for the presence or absence of a distinct hormone or analyte, once daily on a daily basis throughout the period of fertility testing. In an embodiment, at the time of an observed result depicting a 1.5 fold increase on any diagnostic test performed within a single menstrual cycle, a message is delivered via the graphical user interface of the Patient-Facing Application or to another display suggesting that a follicle has matured and/or confirming that the fertile window has opened and remains open. The present inventor has recognized that such information is useful to a subject woman in association with maximizing her chances for conception during a specific menstrual cycle.

In an embodiment, LH testing via a diagnostic test 100 comprising at least a testing zone configured to evaluate an applied bodily fluid for the presence or absence of LH at a threshold should commence on the day following a diagnostic test 100 indicating E3G presence of E3G of at least a specified amount, optionally an amount equivalent to at least a 1.5 fold increase over a previously indicated amount, in a tested urine sample on a diagnostic test 100, and should repeat once daily. Immediately following such result, in an embodiment, the user is instructed to commence utilizing a diagnostic test configured to detect the presence or absence of LH at a threshold once daily on a daily basis. In an embodiment of the invention utilizing a diagnostic test comprising a plurality of testing zones comprising one testing zone and corresponding result indication line configured to detect for the presence of E3G and further comprising another testing zone and corresponding result indication line configured to detect for the presence or absence of LH at a threshold, such instruction is not necessary and therefore not included. In such alternative, it is advantageous instead to instruct the user to simply repeat use of the diagnostic test 100 comprising a plurality of testing zones and corresponding result indication lines, each of which is configured to detect for the presence or absence of a distinct hormone or analyte, once daily on a daily basis throughout the period of fertility testing. In a case where FSH remains elevated for a period of many consecutive days without a result demonstrating a 1.5 fold increase in E3G as compared to a previous result indicated by a diagnostic test 100 previously performed during the same menstrual cycle, a message optionally is delivered to the user (in one example only if the user is above a certain age, i.e. 35 years old) of the system that menopause has likely started. In another case where FSH remains elevated for a period of many consecutive days without a result demonstrating a 1.5 fold increase in E3G as compared to a previous result indicated by a diagnostic test 100 previously performed during the same menstrual cycle, a message optionally is delivered to the user of the system that ovulation is not likely during the present menstrual cycle. Further, a the persistence of many consecutive days (optionally defined as 8 consecutive days) without a result demonstrating a 1.5 fold increase in E3G as compared to a previous result indicated by a diagnostic test 100 previously performed during the same menstrual cycle indicates that the subject woman will not ovulate during the relevant menstrual cycle and that the subject woman is likely infertile during the relevant menstrual cycle, and such messages are optionally generated and displayed in association with the teachings herein. A message optionally is delivered via the graphical user interface to the user of the system following a positive result for the presence of E3G, the term “positive result for the presence of E3G” optionally defined as a sequence of diagnostic tests 100 performed within the same menstrual cycle demonstrating at least a 1.5 fold increase in E3G, that the follicle has matured and/or that the fertile window has opened. An example of such a message is provided in FIG. 9.

Further in accordance with such sequence and teachings, a fifth step comprises following the earlier of an evidenced at least 1.5 fold increase in the level of E3G in the tested bodily fluid within a series of previously taken diagnostic tests 100 in the same menstrual cycle or the passage from the onset of menstruation in the same menstrual cycle of a quantity of days equivalent to the number of days corresponding to the length of the subject woman's average menstrual cycle less 18 days, commencing testing for LH by utilization of a diagnostic test 100 configured to detect at least the presence or absence of LH at a threshold once daily on a daily basis 3005. Following a result indicating the presence of E3G at a threshold in a tested urine sample on a diagnostic test 100, the user is instructed to commence utilizing a diagnostic test 100 configured to detect the presence or absence of LH at a threshold once daily on a daily basis. In an embodiment of the invention utilizing a diagnostic test 100 comprising a plurality of testing zones comprising one testing zone and corresponding result indication line configured to detect for the presence of E3G and further comprising another testing zone and corresponding result indication line configured to detect for the presence or absence of LH at a threshold, such instruction is not necessary and therefore not included. In such alternative, it is advantageous instead to instruct the user to simply repeat use of the diagnostic test 100 comprising a plurality of testing zones and corresponding result indication lines, each of which is configured to detect for the presence or absence of a distinct hormone or analyte, once daily on a daily basis throughout the period of fertility testing. A message optionally is delivered via the graphical user interface to the user of the system following the positive result for the presence of E3G, optionally as evidenced by an indication of at least a 1.5-fold increase in the level of E3G, that the follicle has matured and/or that the fertile window has opened. In a case where FSH remains elevated for a period of many consecutive days without a positive E3G result, a message optionally is delivered to the user of the system that menopause has likely started.

Further in accordance with such sequence and teachings, a sixth step comprises repeating the testing for LH once daily with a series of subsequently performed lateral flow assays each comprising at least a testing zone and corresponding result indication line configured to evaluate for the presence or absence of LH at a threshold until a single lateral flow assay in the series indicates a positive result for the presence of LH at a threshold 3006.

Following a result indicating the presence of LH at a threshold in a tested urine sample on a diagnostic test, the user is instructed to commence utilizing a diagnostic test configured to detect the presence or absence of PdG at a threshold once daily on a daily basis. In an embodiment of the invention utilizing a diagnostic test comprising a plurality of testing zones comprising one testing zone and corresponding result indication line configured to detect for the presence or absence of LH at a threshold and further comprising another testing zone and corresponding result indication line configured to detect for the presence or absence of PdG at a threshold, such instruction is not necessary and therefore not included. In such alternative, it is advantageous instead to instruct the user to simply repeat use of the diagnostic test comprising a plurality of testing zones and corresponding result indication lines, each of which is configured to detect for the presence or absence of a distinct hormone or analyte, once daily on a daily basis throughout the period of fertility testing. A message optionally is delivered via the graphical user interface to the user of the system following the positive result for the presence of LH in urine that ovulation is imminent and/or that fertility may be at its maximum level. As an example, such information is useful to the subject woman in association with timing sexual intercourse to maximize chances for conception.

Following a result indicating the presence of PdG at a threshold in a tested urine sample on a diagnostic test, the user is instructed to commence utilizing a diagnostic test configured to detect the presence or absence of PdG at a threshold once daily on a daily basis. A message optionally is delivered via the graphical user interface to the user of the system following the positive result for the presence of PdG in urine that ovulation has occurred and/or that the user's infertile phase has commenced and/or that the risk of pregnancy is low or decreased until menstruation begins. In an example, the present inventor has recognized that generally progesterone production occurs at a timeframe equivalent to the length of the woman's average menstrual cycle less 10 days following the onset of menstruation. Therefore, it is useful to commence testing for PdG in a menstrual cycle at this point if a positive result for LH has not occurred within this timeframe. In an example, if no diagnostic tests in the menstrual cycle have indicated a positive result for the presence of LH at a threshold, thereby indicating a persistently low level of LH, a message is delivered via the graphical user interface of the Patient-Facing Application or via another display suggesting that ovulation may not occur or may otherwise be insufficient in this menstrual cycle for the subject woman to conceive. Following a single result on a diagnostic test indicating the presence of PdG at a threshold selected from the range of 1 μg/mL-10 μg/mL, which the present inventor has recognized corresponds to the amount of PdG in urine following the production of progesterone in association with and thereby confirmatory of ovulation in the vast majority of women, a message is delivered via the graphical user interface of a Patient-Facing Application or to another display suggesting that the infertile period has commenced and/or that the risk of pregnancy is low or decreased until menstruation begins. In an exemplary case where a plurality of diagnostic tests configured to evaluate urine for the presence or absence of PdG at a thresholds during the period inclusive of 7-10 days past ovulation each indicates the presence of PdG at the threshold, a message is delivered via the graphical user interface that the subject woman has successfully ovulated, or alternatively that the woman has sufficiently ovulated. The present inventor has recognized that such information is useful to a subject woman who wishes diagnose problems associated with ovulation, such as PCOS. In an alternative embodiment, such as an embodiment configured to evaluate a fluid other than urine, as an alternative to testing for PdG it may be advantageous to test the fluid for progesterone, and the relevant messages may be delivered in association with the teachings described elsewhere herein. It is a teaching of embodiments of the invention to test bodily fluids other than urine (such as sweat or serum) for PdG in association with the methods described herein, as PdG remains elevated in such bodily fluids resulting from elevated serum progesterone. The present inventor has recognized that such information is useful to a subject woman who wishes to avoid pregnancy.

Further in accordance with such sequence and teachings, a further method step comprises following the earlier of the occurrence of a lateral flow assay indicating a positive result for the presence of LH at a threshold within a series of previously taken diagnostic tests within the same menstrual cycle or the passage from the onset of menstruation in the same menstrual cycle of a number of days corresponding to the length of the subject woman's average menstrual cycle less 10 days, commencing testing for PdG by utilization of a diagnostic test configured to detect at least the presence or absence of PdG at a threshold once daily on a daily basis 3007.

The present inventor has also recognized the uniquely enabling attributes of the PdG test described herein and to the various references incorporated herein to which priority is claimed. It is therefore a critical step in one embodiment that comprises determining a result, the result at least comprising an indication for the presence or absence of pregnanediol glucuronide in the woman's urine via an indication generated by a lateral flow assay test comprising at least one testing zone and corresponding result indication line configured to detect for the presence or absence of pregnanediol glucuronide in urine at a threshold selected from within the range of 1 μg/mL-10 μg/mL 4001.

Further steps, as enabled in accordance with the descriptions herein, comprise the following:

Allocating the result from the lateral flow assay test configured to detect for the presence or absence of at least pregnanediol glucuronide in urine to the specific calendar date upon which the result was determined 4002. Such step optionally takes place in accordance with or by otherwise utilizing the Calendar and/or the Patient-Facing Application as described elsewhere herein.

Storing the result and the calendar date associated with the result 4003. Such step optionally takes place in accordance with or by otherwise utilizing the Calendar and/or the Patient-Facing Application as described elsewhere herein.

Associating and storing a specific calendar date with the ovulation date of the subject woman 4004. In an example, the subject woman may manually input her ovulation date. In various embodiments, the ovulation date is estimated in accordance with the teachings elsewhere herein. Such step optionally takes place in accordance with or by otherwise utilizing the Calendar and/or the Patient-Facing Application as described elsewhere herein.

Depicting the result onto a display 4005. Such step optionally takes place in accordance with or by otherwise utilizing the Calendar and/or the Patient-Facing Application as described elsewhere herein. In an embodiment, the display 605 is configured as depicted by FIG. 10b or FIG. 10d.

Determining a secondary result from a lateral flow assay test comprising for the presence or absence of at least one additional hormone or hormone metabolite selected from the group consisting of lutenizing hormone, estrogen, and human chorionic gonadotropin, each at a specific threshold.

Allocating the secondary result from the lateral flow assay test configured to detect at least one additional hormone or hormonal analyte selected from the group consisting of lutenizing hormone (LH) at a specific threshold, human chorionic gonadotropin (hCG) at a specific threshold, E3G at a quantity corresponding to an indicated color intensity and FSH at a quantity corresponding to an indicated color intensity, the result at least comprising an indication of the absence or presence of LH or hCG or an indication of the presence of E3G or FSH at an amount corresponding to the indicated color intensity, to the specific calendar date upon which the result was determined 4006. Such a lateral flow assay optionally takes the form of the diagnostic test(s), and operates in coordination with the color intensity key 800 and other components as described elsewhere herein.

Associating and storing a specific calendar date with the ovulation date of the subject woman 4007. Such step optionally takes place in accordance with or by otherwise utilizing the Calendar and/or the Patient-Facing Application as described elsewhere herein.

Depicting the results onto a display 4008. Such step optionally takes place in accordance with the display 605 depicted by FIG. 10b or FIG. 10d, or by otherwise utilizing the Calendar and/or the Patient-Facing Application as described elsewhere herein.

Formatting each result from the lateral flow assay test in combination with individually identifying information associated with the subject woman and the date the test was performed into formatted results for interoperable transfer to a computing device configured to interpret and store electronic personal health information 4009. Such step optionally takes place in accordance with or by otherwise utilizing the Patient-Facing Application as described elsewhere herein.

Transferring each collected result in an interoperable format either individually or in combination with one or more additional results associated with the same subject woman 4010. Such step optionally takes place in accordance with or by otherwise utilizing the Patient-Facing Application, the Healthcare Professional-Facing Application and/or the Telemedicine System as described elsewhere herein.

Identifying one or more trends associated with the undesirable absence of at least one hormone or hormonal analyte during a specified timeframe as indicated by a plurality of stored results each generated by a lateral flow assay test 4011. Such step optionally takes place in accordance with or by otherwise utilizing the Fertility Tracking System as described elsewhere herein.

Determining a prescribed action plan to correct the undesirable absence of at least one hormone or hormonal analyte or associated trends 4012. Such step optionally takes place in accordance with or by otherwise utilizing the Seed Consumption System as described elsewhere herein.

Communicating the prescribed action plan to the subject woman 4013. Such step optionally takes place in accordance with or by otherwise utilizing the Patient-Facing Application as described elsewhere herein. Optionally, the prescribed action plan comprises a specified suggested change in diet, specifically commencing the once daily consumption of pumpkin seeds optionally in the amount of 1 tablespoon and flax seeds optionally in the amount of 1 tablespoon, and optionally in snack bar form, once daily upon the start of menstruation, or optionally once daily following the first indication in a single menstrual cycle for the presence of FSH on a diagnostic test 100 performed on the subject woman's urine, and then subsequently changing to instead engage in daily consumption of sesame seeds optionally in the amount of 1 tablespoon and sunflower seeds optionally in the amount of 1 tablespoon, and optionally in snack bar form, following the first indication of a positive LH result on a diagnostic test 100 performed on the subject woman's urine, or optionally in accordance with or by otherwise utilizing the Seed Consumption System as described elsewhere herein.

Suggesting a telemedicine consultation with a healthcare professional following the identification of one or more trends associated with the undesirable absence of at least one hormone or hormonal analyte as indicated on one or more appropriately configured diagnostic test(s) during a specified timeframe 4015. Such step optionally takes place in accordance with or by otherwise utilizing the Telemedicine System as described elsewhere herein. The one or more such trends may consist of the undesirable absence of pregnanediol glucuronide as indicated by the stored results as indicated on one or more appropriately configured diagnostic test(s) associated with any of the dates occurring from 7-10 days past the subject woman's ovulation date. Such step optionally takes place in accordance with or by otherwise utilizing the Fertility Testing System as described elsewhere herein.

The determining a result step takes place in various embodiments by photographing, via a camera integrated within a smartphone, the diagnostic test 4020. Such step optionally takes place in accordance with or by otherwise utilizing the Patient-Facing Application, Camera and/or Smartphone as described elsewhere herein.

Orienting the photograph of the diagnostic test to determine the locations of the one or more result indication lines of the diagnostic test 4021, optionally by first identifying the end 111 of the diagnostic test 100 and aligning it with a graphical user interface element. Such step optionally takes place in accordance with or by otherwise utilizing the Camera and smartphone 600 as described elsewhere herein.

Identifying the specific color intensity, optionally a specific color intensity correlating to a specific level of analyte and/or hormone as described elsewhere herein, associated within the color detected by the camera within a result indication line of the lateral flow assay test 4022. Such step optionally takes place in accordance with or by otherwise utilizing the Fertility Testing System as described elsewhere herein.

Comparing the intensity of the color detected by the camera within the result indication line of the diagnostic test 100 with a pre-defined threshold intensity, optionally in association with a color intensity key 800, associated with the presence or absence of the hormone or analyte at a specific concentration to generate a result comprising an estimate of the concentration of the hormone or analyte and optionally an interpretation of what such concentration of the hormone or analyte signifies 4023, as further described elsewhere herein. Such step optionally takes place in accordance with or by otherwise utilizing the Fertility Testing System as described elsewhere herein.

The diagnostic test comprising a lateral flow assay test may consist of a single test, such as the diagnostic test as described elsewhere herein, configured to simultaneously or near-simultaneously detect for the presence or absence of a plurality of hormones or hormonal analytes selected from the group consisting of pregnanediol glucuronide, luteinizing hormone, estrogen, estradiol, progesterone and human chorionic gonadotropin. The method may further comprise triggering a delivery of additional lateral flow assay tests following the usage of a quantity of lateral flow assay tests correlative to one menstrual cycle 4024. Such step optionally takes place in accordance with or by otherwise utilizing the Fertility Testing System as described elsewhere herein.

In an example the method further comprises depicting the results and/or interpretations onto a display, optionally via a graphical user interface, featuring a calendar 5000 with the result of each test displayed on in association with the date each diagnostic test was performed within the displayed calendar 4025. Such step optionally takes place in accordance with or by otherwise utilizing the Calendar and graphical user interface as described elsewhere herein.

In various examples of the invention, it is advantageous to overlap testing for multiple hormones and/or analytes from within the same collected urine sample. For example, following a positive result indicated on a diagnostic test for the presence of E3G at a threshold, it remains advantageous to persist in monitoring E3G levels on a daily basis by use of additional diagnostic tests to confirm a positive result of the presence of E3G at a threshold in urine coinciding with a first positive result of LH at a threshold in urine. Such indication of a simultaneous positive result for E3G at a threshold in urine on a diagnostic test and positive result for LH at a threshold in urine on a diagnostic test from the same urine sample suggests that a mature follicle has been developed. Therefore, in an embodiment, such a result generates a message to the user via the graphical user interface that a mature follicle has developed. In contrast, a negative result indicated on a diagnostic test for the presence of E3G at a threshold in urine and a positive result indicated on a diagnostic test for the presence of LH at a threshold in urine could signify a false LH surge or the release of an immature follicle and poor ovulation quality. Therefore, in an embodiment, such a result triggers the generation of a unique message 501 to the user via the graphical user interface comprising of one or more statements selected from the group consisting of: “an immature follicle has developed,” “there is an elevated likelihood of poor ovulation quality,” and “there is a likelihood of false positive result for LH and that LH testing should continue.”

As described in more detail elsewhere herein, it is a teaching of an embodiment that a message or several messages may be facilitated and delivered via other components of the system as described herein, in particular including the graphical user interface of the Patient-Facing Application. In various embodiments, in association with the methods of utilization herein, it is a teaching of an embodiment to combine the Patient-Facing Application with a specific quantity of diagnostic tests each configured to evaluate for the presence or absence of one or a plurality of hormone(s) and/or analyte(s) in such a way to facilitate easy performance of the methods of utilization as described herein. In various embodiments, the results of each test and the calendar date associated with each result is stored for use in association with the calendar 5000 and other elements of the graphical user interface, Patient-Facing Application and Healthcare Professional-Facing Application as described elsewhere herein.

The present inventor has recognized the specific problems associated with poor ovulation quality and that ovulation quality may be measured via a measurement of a urine metabolite of estrogen (such as E3G), as the more mature a follicle is, the more estrogen will be produced. In an embodiment, in the scenario where a positive result is indicated on a diagnostic test for the presence of LH at a threshold in urine and a negative result is indicated on a diagnostic test for the presence of E3G at a threshold resulting from the evaluation of the same sample of urine with one or more diagnostic tests as described herein, it may signify that a follicle did not mature and that an immature egg incapable of fertilization was released during the relevant cycle. In such scenario, in an example of the invention, a message is delivered via the graphical user interface providing notice of the poor ovulation quality, optionally stating that a follicle did not mature and/or that an immature egg incapable of fertilization has been released during this cycle. Also following such result, a prompt to schedule and conduct a telemedicine appointment with a healthcare professional for further evaluation is optionally generated via the graphical user interface in an exemplary embodiment. The threshold ranges associated with the diagnostic tests to evaluate for the presence or absence of the estrogen metabolite (i.e. E3G) and LH as described elsewhere herein correlate to the values that suggest whether a mature egg or an immature egg has been released, and as such, constitutes a teaching of an aspect of the invention.

It is a teaching of the an embodiment to either test the same urine sample with different diagnostic tests, each configured to detect a separate hormone or analyte at a threshold, or to test the same urine sample with a single diagnostic test configured to detect for the presence or absence of a plurality of hormones and/or analytes each at a separate threshold in accordance with the methods of use described herein. Various embodiments of the system described herein incorporate quantities of individual diagnostic tests each configured to detect a separate hormone or analyte at a threshold, or a quantity of diagnostic tests configured to detect for the presence or absence of a plurality of hormones and/or analytes each at a separate threshold, needed to accomplish the mechanisms described herein over a single menstrual cycle, optionally corresponding to the typical number of days in a single menstrual cycle averaged over a typical population. In an embodiment, the system comprises a number of diagnostic tests needed to perform the methods described herein over a plurality of menstrual cycles. The diagnostic testing, message generation, display and other aspects of the performed methods as described are conducted in accordance with the teachings of the methods and apparatuses as described elsewhere herein. Various methods incorporate the step of determining a result from a the lateral flow assay test described elsewhere herein comprising testing zones and corresponding result indication lines each configured to evaluate for the presence or absence of pregnanediol glucuronide and at least one additional hormone or hormonal analyte selected from the group consisting of luteinizing hormone, estrogen, and human chorionic gonadotropin, each hormone or analyte tested at a specific threshold.

In embodiments of the invention, the above sequences are performed without the assistance of a computing device or graphical user interface, with tracking performed with the assistance of a printed calendar included with the system as described elsewhere herein. In an embodiment, instructions for how to perform the sequence as described herein are printed and included in association with the diagnostic tests optionally on the Diagnostic Test Key, and a printed calendar and optionally a subset of other components of the system described herein.

Various embodiments are configured for visual reading or for reading with the assistance of external reading and tracking mechanisms. The external reading and tracking mechanisms may comprise those described elsewhere herein. The external reading and tracking mechanisms may take forms such as those described in PCT Patent Application PCT/US2019/038173 filed on Jun. 20, 2019; U.S. patent application Ser. No. 16/302,085 filed on May 19, 2017; and U.S. patent application Ser. No. 14/505,083 filed on Oct. 2, 2014; each incorporated by reference herein. Such embodiments of the system may also incorporate or interact with the mechanisms for recording, storing and communicating the collected diagnostic test results as described in the above-mentioned or similar references.

The fertility tracking system in certain configurations is more precisely described as a “predicting fertile window system.” The present inventor has recognized that aspects of the invention described herein provide unique benefits to persons wishing to enhance the likelihood of conception by more clearly identifying the opening date and closing date of the fertile window, as indicated by the presence of certain hormones and analytes in urine. In one example, the system is configured to provide prompts via the graphical user interface not only signaling that a certain hormone is present or absent in a sample evaluated with a diagnostic test as described herein, but also an interpretation of the relevance of that hormone or analyte to the certain user. For instance, in a configuration intended to assist a woman to become pregnant, the detection of E3G or FSH in urine at a threshold in association with the utilization of an appropriately configured diagnostic test as described herein may prompt the system to display a message indicating that the subject woman's fertile period has begun and that she should engage in intercourse to achieve pregnancy.

In an example, diagnostic tests configured to detect for the presence or absence of hCG at a threshold, optionally in association with the detection of other hormones in a single test, are utilized in association with the predicting fertile window system. In such diagnostic tests, a result indicating the presence of hCG at a threshold indicates pregnancy. Likewise in such diagnostic tests, a result indicating the absence of hCG at a threshold indicates that the subject woman is not pregnant. Therefore, in an example, following such result, a message is displayed in the graphical user interface that pregnancy has been achieved and that the woman can optionally cease testing, or continue testing especially for PdG on an ongoing basis to ensure that the woman's progesterone levels remain sufficient to support a pregnancy (as indicated by a positive PdG result on a diagnostic test as described herein). In a situation where a diagnostic test indicates a positive result for hCG at a threshold in a tested bodily fluid and testing of the same sample of bodily fluid indicates a result of the absence of PdG at a threshold, an interpretation comprising an indication that the subject woman has likely not produced enough progesterone to sustain pregnancy. Likewise, in a situation where a diagnostic test indicates a positive result for hCG at a threshold in a tested bodily fluid and testing of the same sample of bodily fluid indicates a result of the presence of PdG at a threshold, an interpretation comprising an indication that the subject woman has likely produced enough progesterone to sustain pregnancy. In various embodiments messages intended to convey the above results and/or interpretations are delivered via components of the system as described elsewhere herein.

In an embodiment, the system described herein is configured as a system to predict the fertile window of a patient user. In one aspect, the system may be configured for use with both diagnostic tests configured to detect for the presence or absence of PdG at a threshold in urine and separately diagnostic tests configured to detect for the presence or absence of LH at a threshold in urine. In another aspect, the system may be configured for use with diagnostic tests configured to detect for at least both the presence or absence of PdG at a threshold in urine and for the presence or absence of LH at a threshold in urine within a single diagnostic test. In accordance with the predicting fertile window system, it may be advantageous for the user to perform the diagnostic test at least once daily throughout the course of the menstrual cycle.

Likewise, the present inventor has recognized that aspects of the invention described herein provide unique benefits to persons wishing to avoid conception and avoid pregnancy by more clearly identifying especially the closing date of the fertile window. In an embodiment, the system described herein is configured as a system to allow a patient user to avoid pregnancy. In such embodiment, the present inventor has identified a context for use of the system as a form of birth control. In one example, the system is configured to provide prompts via the graphical user interface not only signaling that PdG is present at a threshold correlating to ovulation in a sample evaluated with a diagnostic test as described herein, but also an interpretation of the relevance of the presence of PdG in the sample to a user, namely that she has ovulated and that her infertile period has begun or that she may engage in sexual intercourse without the risk of becoming pregnant. Thus, it is a teaching of an example of the invention to confirm ovulation, which is applied as an aid to the avoidance of unwanted pregnancy.

It is a teaching in association with the predicting fertile window system to trigger the recurring purchase and delivery of a quantity of diagnostic tests configured to detect both the absence or presence of PdG at a threshold and the presence or absence of LH at a threshold in urine following a period correlating to a woman's menstrual cycle, optionally via Amazon, and optionally as a component of the Patient-Facing Application. The present inventor has recognized that the system configured to trigger such purchase and delivery on a recurring basis provides value especially to women who wish to avoid pregnancy by ensuring a supply of the diagnostic tests as further described herein needed for utilization in association with teachings of the system.

Telemedicine System

An embodiment of the invention is configured as a telemedicine system. In such configuration, the system comprises the Scheduler as described elsewhere herein, one or more Diagnostic Tests as described elsewhere herein, the Computing Device, Camera, Display, Graphical User Interface, Communicatively Connected Storage Medium and Processor each as described elsewhere herein, a Patient Information Integration Tool, a Healthcare Professional-Facing Application and a Patient-Facing Application.

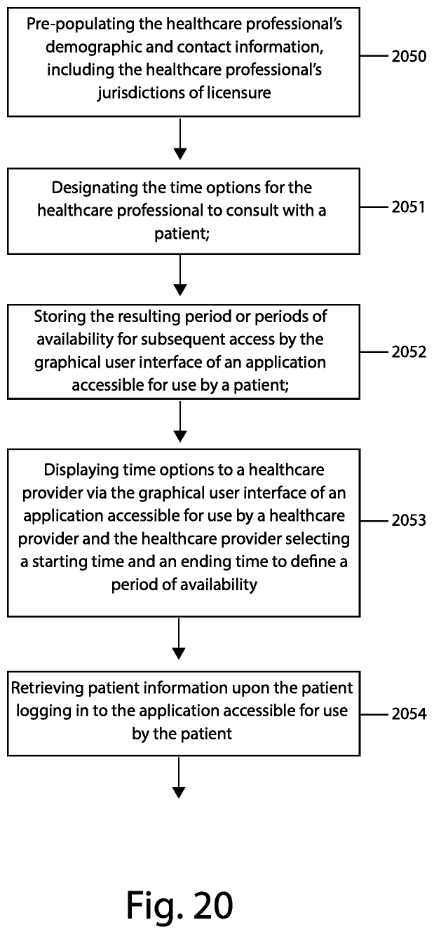

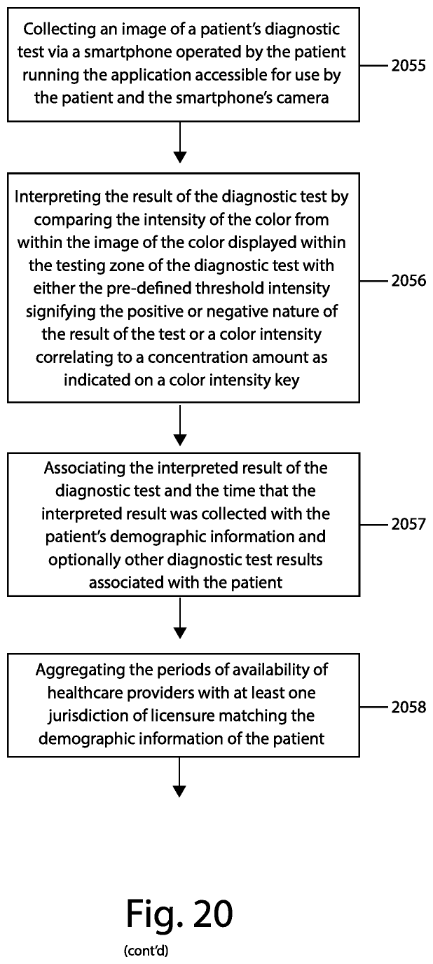

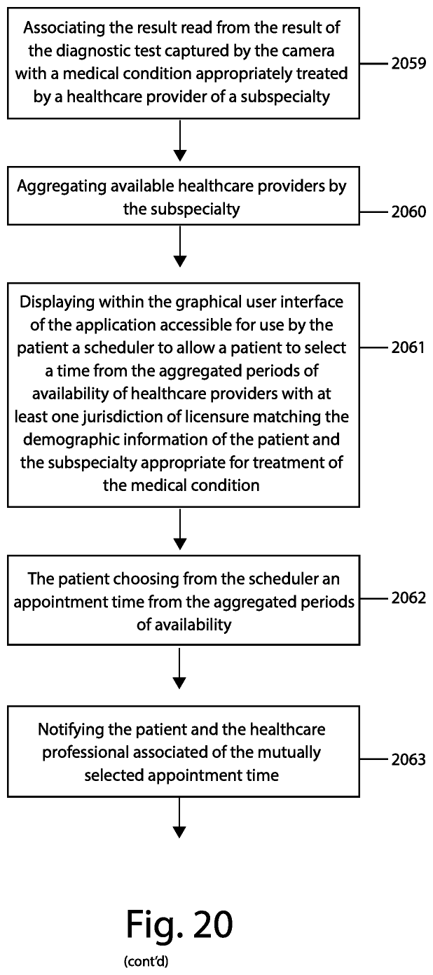

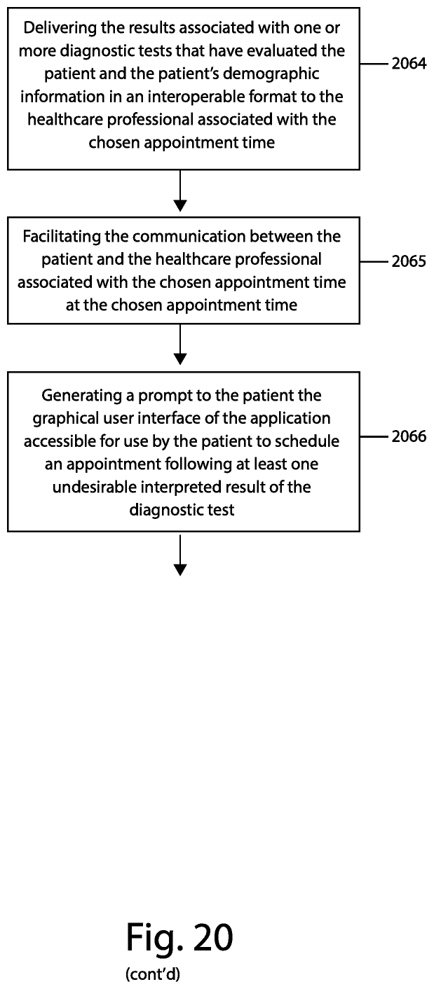

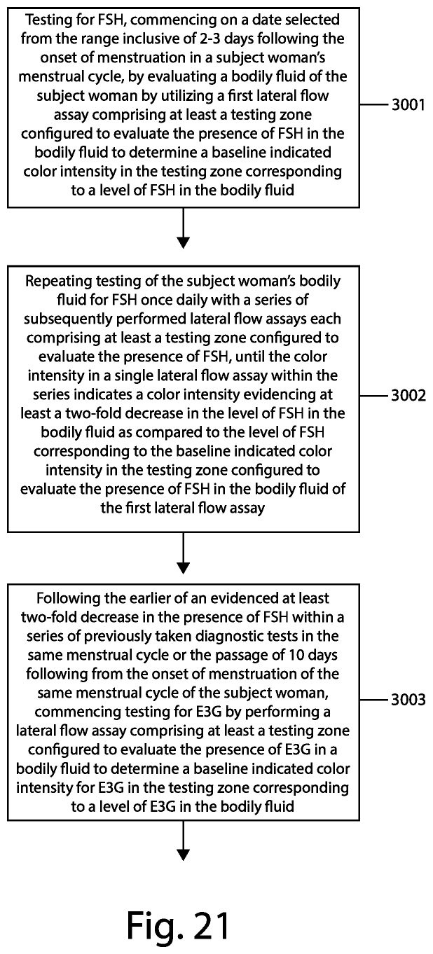

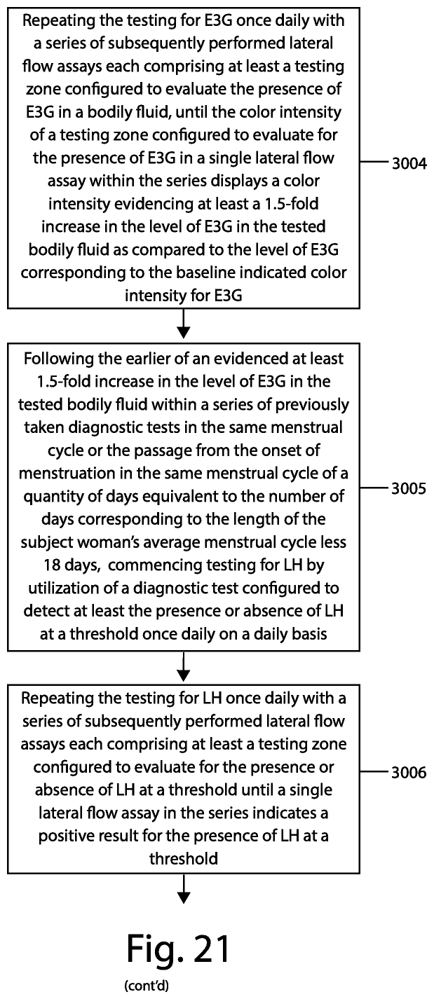

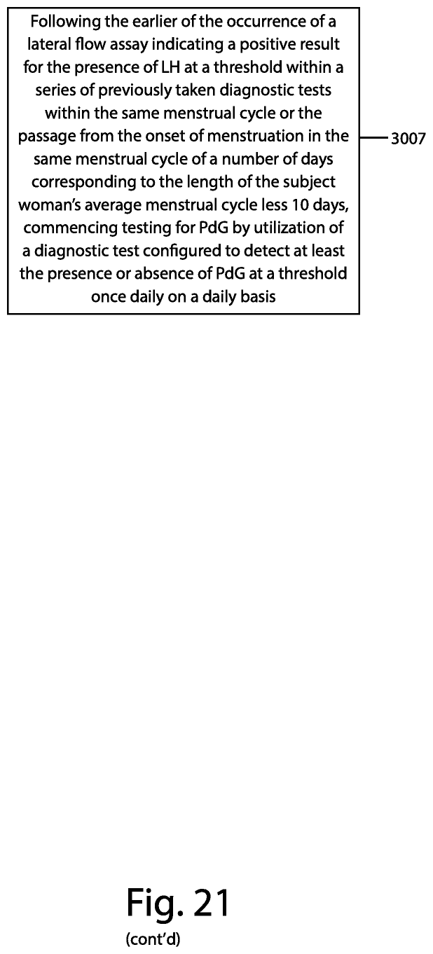

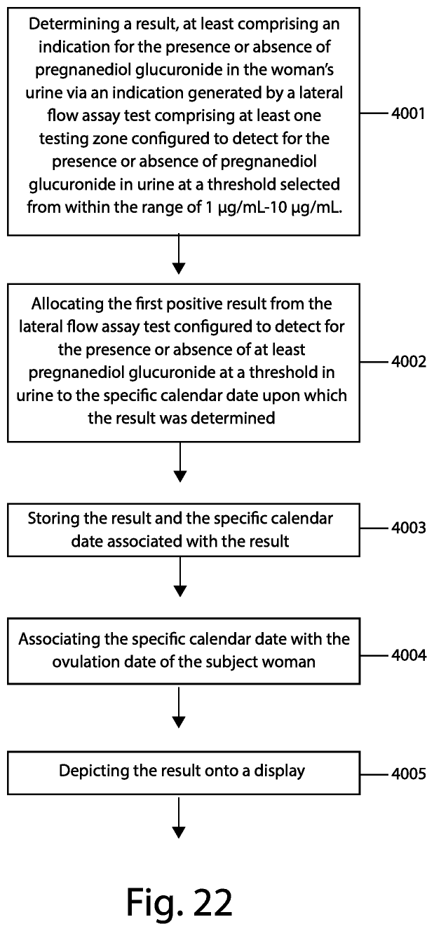

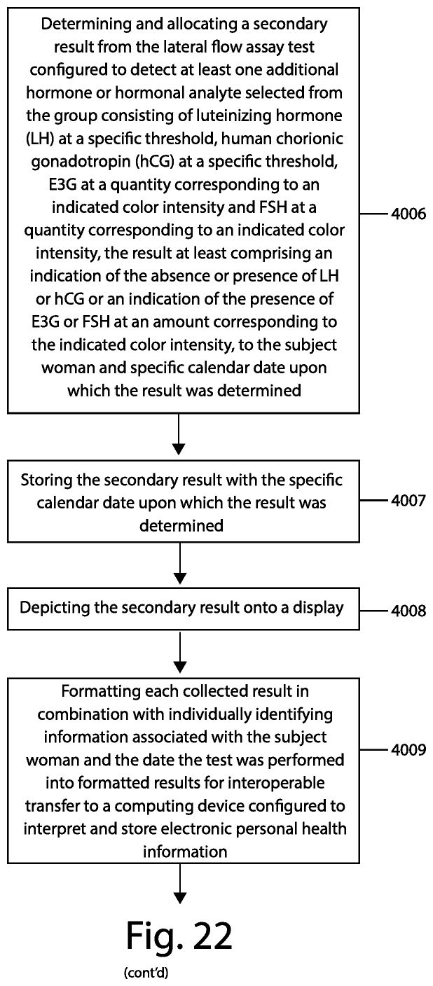

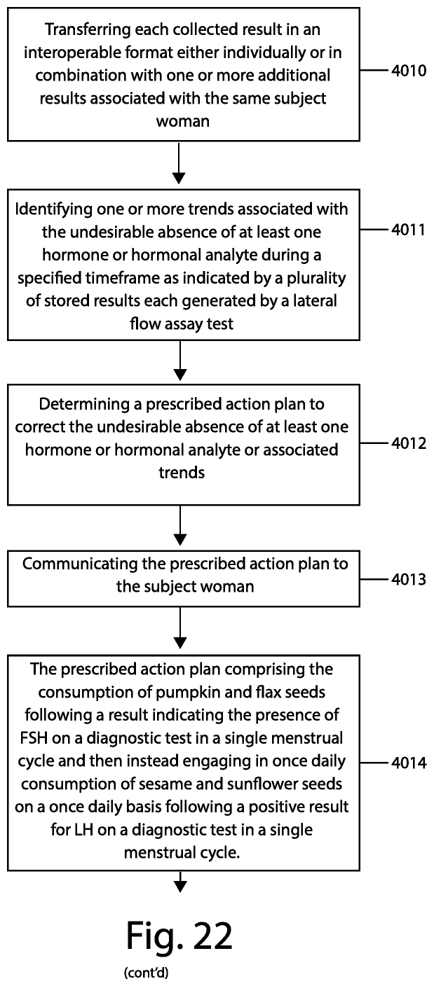

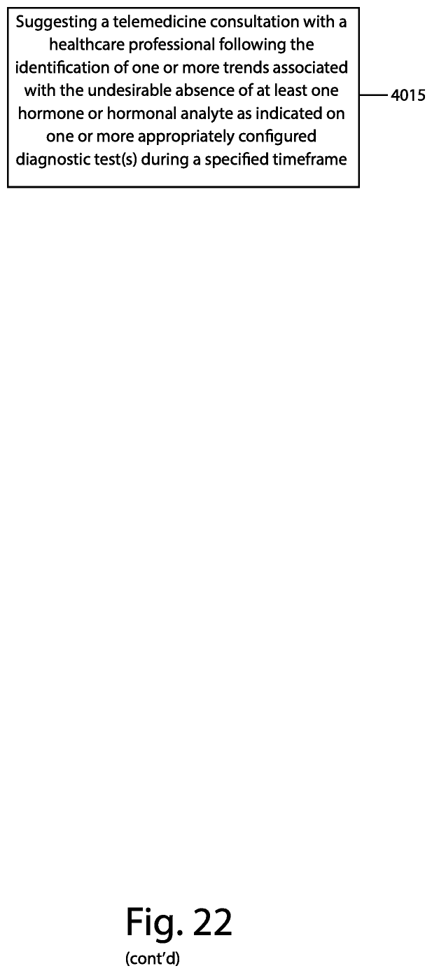

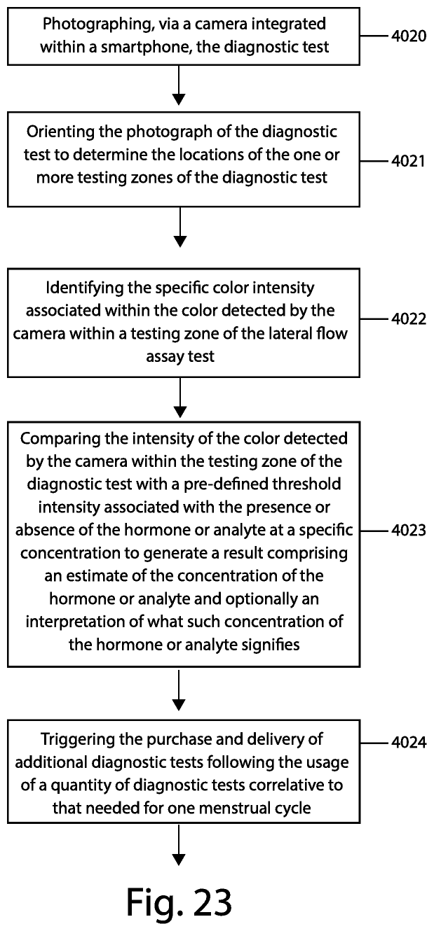

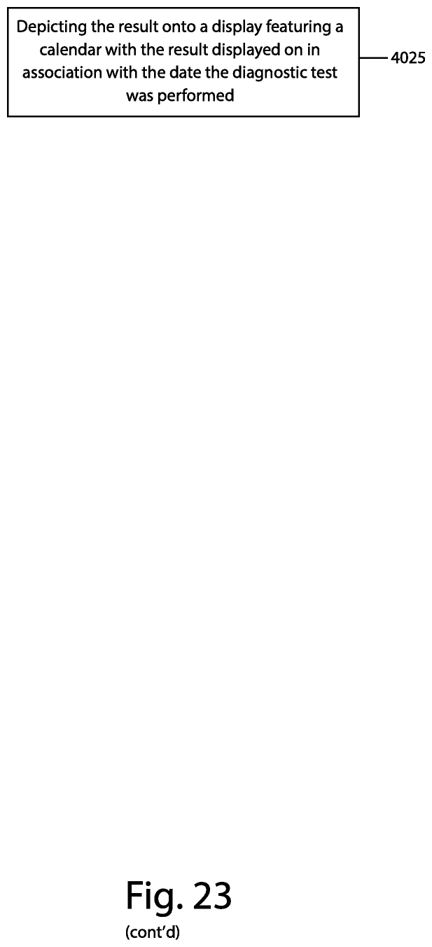

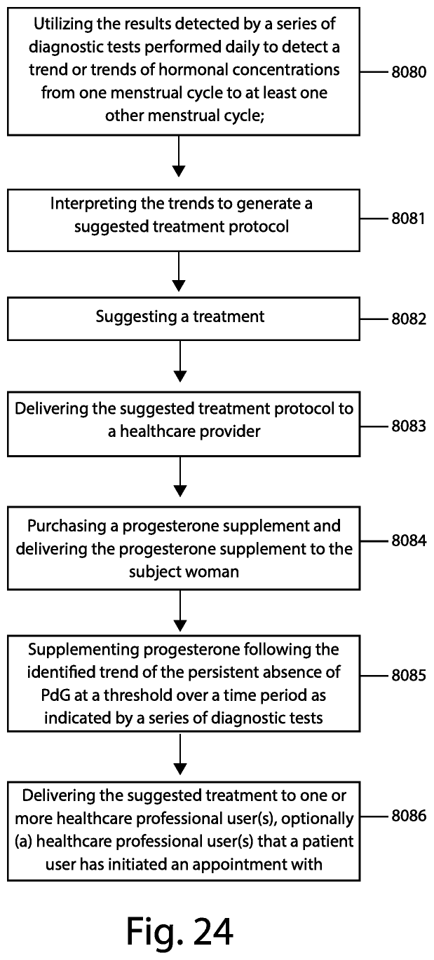

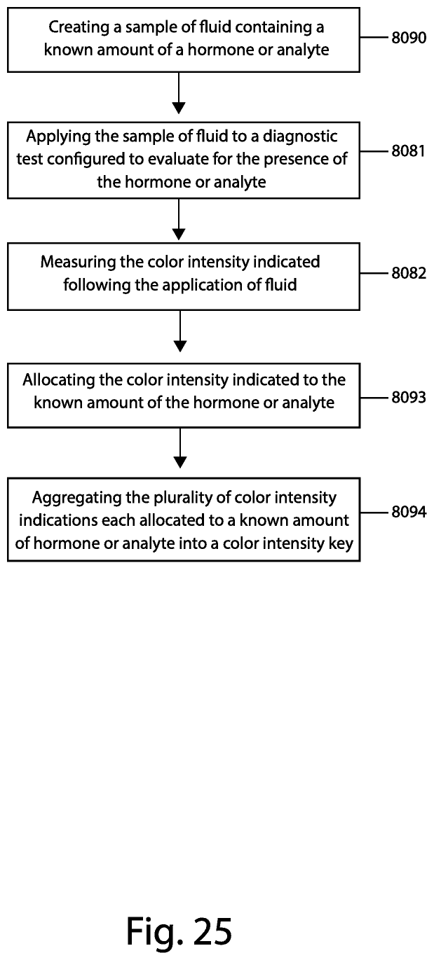

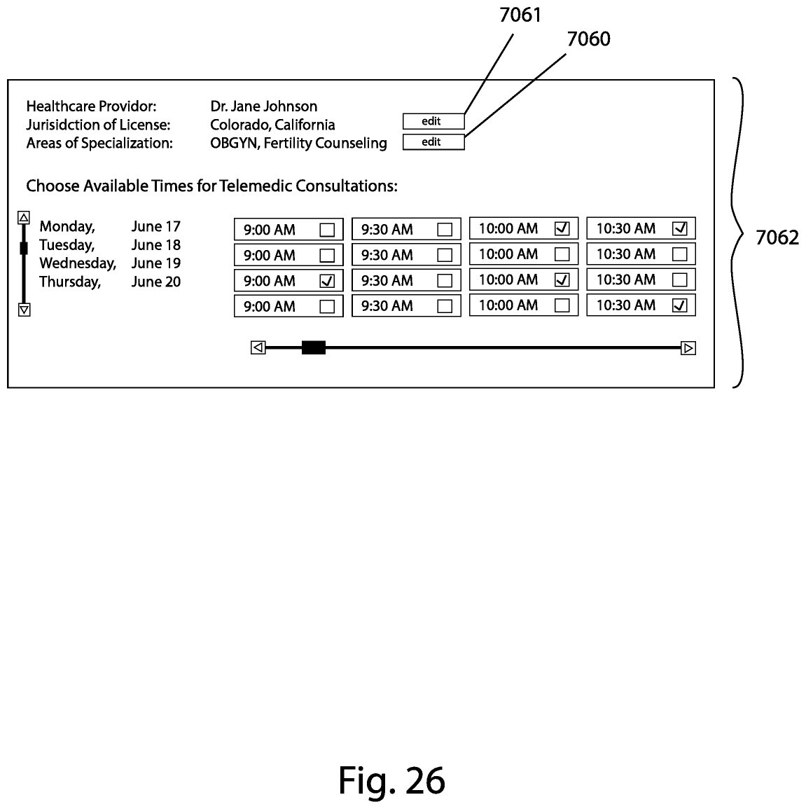

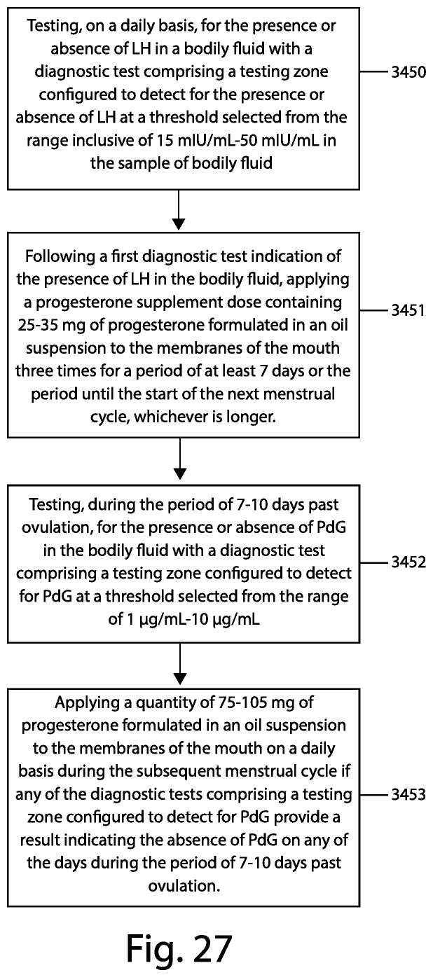

In association with the telemedicine system, the Healthcare Professional-Facing Application, as illustrated in FIG. 20, is configured to provide users who are healthcare providers with the ability to indicate the conditions that they have the capability to treat via the graphical user interface 7060 and optionally other aspects of the system. The Healthcare Professional-Facing Application, as illustrated in FIG. 20, is further configured to provide users who are healthcare providers with the ability to indicate the jurisdictions that they are licensed within, or otherwise have the capability to practice within, via the graphical user interface 7060 and optionally other aspects of the system. In an example, the Healthcare Professional-Facing Application, upon the healthcare provider's initial use of the system, requires the healthcare provider to enter their national provider identifier (NPI) to facilitate the confirmation of licensure information, optionally configured to occur automatically following the healthcare provider's entry of his or her NPI via via an application program interface (API). For instance, a healthcare provider possessing the capability, as determined by training and licensure, for example, to treat infertility may so associate such ability in association with their profile. In the case where a subject woman has captured a diagnostic test via a smartphone camera and a Patient-Facing Application indicating infertility, it is an object of the telemedicine system to facilitate a consultation among the subject woman and the qualified healthcare provider. To achieve this objective, in an example, the Healthcare Professional-Facing Application is configured to allow the healthcare provider to choose his or her available times for a consultation via a graphical user interface 7062. In an example, the times of all available healthcare providers licensed in a patient's jurisdiction capable of treating the patient's indicated condition are aggregated and displayed to the patient via the Patient-Facing Application without depicting the name or any other specifically identifying information associated with the available healthcare providers 900, as depicted in FIG. 12. The present inventor has recognized the value in anonymously assembling a group of healthcare providers and organizing them merely by jurisdictional licensure and subspecialty for display to a patient, as it provides a more efficient mechanism for connecting a plurality of patients with a plurality of healthcare providers in advance of a telemedicine consultation, which forms a step of a method embodiment of the invention. In one example, the plurality of healthcare providers are further vetted for reviews, lack of complaints and other qualifications prior to being granted the ability to select available times to provide telemedicine consultations in association with the system. In association with foregoing, and as enabled by utilization of the other components of the system as described herein, a method embodiment comprises the steps of assembling a group of healthcare providers by collecting their jurisdiction of licensure and subspecialty via a graphical user interface, optionally the graphical user interface associated with the Healthcare Provider-Facing Application as described elsewhere herein, displayed to each of the group of healthcare providers 7080; collecting, via the graphical user interface displayed to each of the group of healthcare providers, the times available to schedule a telemedicine consultation of each of the group of healthcare providers 7081; storing information, comprising the jurisdiction of licensure, subspecialty, and available appointment times, of each of the group of the healthcare providers in a communicatively connected storage medium 7082; accessing the information with an application, optionally the Patient-Facing Application, further configured to access the communicatively connected storage medium and retrieve the jurisdictions of licensure, subspecialty, and available appointment times of each of the group of the healthcare providers 7083; determining the location of a patient utilizing the application by retrieving the location from the GPS of the device, optionally the smartphone 600, operating the application 7084; interpreting an indication of at least one diagnostic test 100, optionally a diagnostic test featuring at least one testing zone and corresponding result indication line configured to evaluate for the presence or absence of pregnanediol glucuronide at a threshold as described elsewhere herein, that has evaluated a bodily fluid of the patient to retrieve a medical condition of the patient relevant to at least one subspecialty and determine the subspecialty, which in an example defaults to reproductive medicine based on the choice of diagnostic tests utilized in association with the method to evaluate for the presence of any of hCG, PdG, LH, FSH or E3G, relevant to the medical condition of the patient 7085; filtering the times available to conduct a telemedicine consultation by aggregating only those relevant to the subset of one or more healthcare providers whose jurisdiction of licensure matches the location of the patient and whose subspecialty is relevant to the medical condition of the patient 7086; depicting onto the graphical user interface of the application for display to the patient, anonymously, the times available to conduct a telemedicine consultation with at least one healthcare provider from the subset of healthcare providers whose jurisdiction of licensure matches the location of the patient and whose subspecialty is relevant to the medical condition of the patient 7087, as depicted in FIG. 12; and the patient choosing a time from those depicted onto the graphical user interface 7088. The patient and the healthcare provider may then engage in a telemedicine consultation at the chosen time as described elsewhere herein.

It is also an object of the telemedicine system to allow the qualified healthcare provider to consult with the subject woman and generate a suggested treatment protocol, which may incorporate the provision of a prescription, for example. In an example, the prescription may be for the ingestion, suppository and/or injection of progesterone. The prescription for progesterone in one example is given by the qualified healthcare provider for delivery to the subject woman at a specific time measured by days past ovulation associated with the subject woman's menstrual cycle, determined in association with one or more diagnostic test(s) 100 and the Patient-Facing Application. It is a teaching of the telemedicine system to provide a notification via the Patient-Facing Application reminding the subject woman to receive delivery of the progesterone prescription in at a specific time, optionally immediately following a result of a diagnostic test 100 performed on a fluid sample collected from the subject woman indicating the absence of pregnanediol glucuronide at a threshold.

In one example, the telemedicine system generates a message to a specified qualified healthcare provider via the graphical user interface of the Healthcare Professional-Facing Application and optionally associated notification mechanisms following a result on a diagnostic test 100 collected from a sample of a healthcare provider's patient providing an indication of the absence of pregnanediol glucuronide a threshold on any day during the time period of 7-10 days past the date of ovulation, the date of ovulation determined as described elsewhere herein. Such result is collected and formatted optionally in association with the patient's use of the Patient-Facing Application. It is a teaching of an embodiment that the healthcare provider, following the receipt of a diagnostic test result via the telemedicine system and its associated components, optionally the Healthcare Provider-Facing Application, indicating a level of pregnanediol glucuronide below a threshold on any day during the time period of 7-10 days past ovulation of the subject woman in association with the teachings of the invention, provides, optionally following a suggestion triggered as described elsewhere herein, a prescription to the subject woman, optionally during or after a telemedicine consultation with the subject woman, to ingest, supposit or inject progesterone during the time period of 3-14 days past ovulation of the following cycle, or during an expanded time period. Therefore, in accordance with the teachings associated with the telemedicine system as described herein and other embodiments, steps of the method of use of an exemplary system include determining a prescribed action plan to correct the undesirable absence of at least one hormone or hormonal analyte 2014, and communicating the prescribed action plan to the subject woman 2015 optionally via a consultation facilitated as described herein, or a communication facilitated at least in part via the graphical user interface of the Patient-Facing Application.

It is a teaching of an embodiment that the telemedicine system is configured to store each healthcare professional's demographic and contact information within a Communicatively Connected Storage Medium, optionally configured to store at least the qualified healthcare professional's jurisdictions of licensure. Such information may be gathered by the telemedicine system via the graphical user interface of the Healthcare Professional-Facing Application upon the healthcare professional logging in to an Healthcare Professional-Facing Application and stored in the Communicatively Connected Storage Medium for subsequent retrieval. Upon the qualified healthcare professional logging in to the telemedicine system, such information is then made accessible and editable to the healthcare professional. It is therefore a step of the method of use of the telemedicine system to engage in pre-populating the healthcare professional's demographic and contact information, including the healthcare professional's jurisdictions of licensure, optionally retrieved from the communicatively connected storage medium upon the healthcare professional logging in to the Healthcare Professional-Facing Application 2050.

One additional type of information that the qualified healthcare professional may input into the telemedicine system via the graphical user interface are the available starting and ending time options for the qualified healthcare professional to engage in a consultation with a patient. It is therefore a step of the method embodiment to engage in designating the time options for the healthcare professional to consult with a patient 2051. It is a teaching of an embodiment to store such available starting and ending time options for subsequent utilization by the system, optionally in coordinating consultations with patients. It is therefore a step of the method of use of the telemedicine system to engage in storing the resulting period or periods of availability for subsequent access by the graphical user interface of an application accessible for use by a patient, optionally the Patient-Facing Application 2052. The Healthcare Professional-Facing Application is configured to allow the qualified healthcare professional to provide such information via the associated graphical user interface of the Healthcare Professional-Facing Application. It is therefore a step of the method of use of the telemedicine system to engage in displaying starting and ending time options to a healthcare provider via the graphical user interface of an application accessible for use by a healthcare provider, optionally the Healthcare Professional-Facing Application; the healthcare provider selecting a starting time and an ending time to define a period of availability via the Healthcare Professional-Facing Application 2053.

In association with the Patient-Facing Application, alternatively described as “an application accessible for use by a patient” and as described elsewhere herein, it is a teaching of an embodiment to collect and store information relevant to a layperson user, optionally a patient. In association with such teaching, it is an aspect of the invention to provide a patient information storage system. Such a system, operating in conjunction with the Communicatively Connected Storage Medium, the graphical user interface and other components, is configured to configured to collect and save electronic personal health information, optionally comprising the results of one or more diagnostic tests. In an embodiment, the patient information storage system is interoperable, accessible via API, and compliant with standards and regulations associated with the exchange of electronic personal health information (such as, for instance, HIPAA and the HITECH Act). In one example, the system is configured to allocate non-PACS medical image taken by a layperson, for instance a photograph of a diagnostic test, to the patient information storage system in association with a specified patient user. Relevantly, the system is configured to store and allow for retrieval of the patient's demographic information. It is therefore a step of the method of use of the telemedicine system to engage in retrieving the patient's demographic information upon the patient logging in to a patient-facing application 2054.

As described elsewhere herein, the telemedicine system incorporates a camera configured to collect the results of a patient's lateral flow assay test. In association with such teaching, it is therefore a step of the method of use of the telemedicine system to engage in collecting the image of a patient's diagnostic test via a device operated by the patient, optionally a smartphone 600, running the Patient-Facing Application and the device's camera 2055.

As described elsewhere herein, the system incorporates a processor configured to interpret the result of the diagnostic test in part by evaluating the intensity of the color displayed thereon and comparing such color to another basis color. In association with such teaching, it is therefore a step of the method of use of the telemedicine system to engage in interpreting the result of the diagnostic test 100 by comparing the intensity of the color displayed within the result indication line of the diagnostic test 100 with either the pre-defined threshold intensity signifying the positive or negative nature of the result of the test or a color intensity correlating to a concentration amount as indicated on a color intensity key 800 as described elsewhere herein 2056.

Following the interpretation of a diagnostic test, the components of the system as described herein work to allocate the result to associate with a specified patient. For instance, it is an aspect of the system to provide a Patient Information Integration Tool to instruct the Processor to associate each diagnostic result with a specified person, optionally a person logged in to the Patient-Facing Application at the time a diagnostic test is photographed with the Camera of the associated Computing Device. In association with such teaching, it is therefore a step of the method of use of the telemedicine system to engage in associating the interpreted result of the diagnostic test and the time that the interpreted result was collected with the patient's demographic information and optionally other diagnostic test results associated with the patient 2057.

Following the collection of the time periods of availability of one or more healthcare professionals, optionally via the Scheduler as described elsewhere herein, the components of the system as described herein work to aggregate the periods of availability. Further, it is an aspect of the invention to provide a Patient—Healthcare Provider Integration Tool to display the aggregated periods of availability filtered by jurisdictions of licensure for each healthcare professional matching the locale of an individual patient. In association with such teaching, it is therefore a step of the method of use of the telemedicine system to engage in aggregating the periods of availability of healthcare providers with at least one jurisdiction of licensure matching the demographic information of the patient 2058. Relatedly, the system optionally is configured to detect and interpret the result of a diagnostic test, associate a medical condition to such result, and then filter healthcare professionals who are qualified to treat such medical condition and display their periods of availability. In association with such teaching, it is therefore a step of the method of use of the telemedicine system to engage in associating the result read from the result of the diagnostic test captured by the camera with a medical condition appropriately treated by a healthcare provider of a subspecialty 2059, and aggregating available healthcare providers of that subspecialty for subsequent display 2060.

In association with the Scheduler as described elsewhere herein and various other components of the system (such as, especially, the graphical user interface), it is a teaching of an embodiment to empower a user of the Patient-Facing Application to select a time from the aggregated time periods of availability of one or more healthcare professionals via a display. In association with such teaching, it is therefore a step of the method of use of the telemedicine system to engage in displaying within the interface of the Patient-Facing Application a scheduler to allow a patient to select a time from the aggregated periods of availability of healthcare providers with at least one jurisdiction of licensure matching the demographic information of the patient, and optionally the subspecialty appropriate for treatment of the medical condition 2061. In association with such teaching, it is also therefore a step of the method of use of the telemedicine system for the patient to engage in choosing from the scheduler an appointment time from the aggregated periods of availability 2062.

It is a teaching of the system to notify the relevant healthcare professional and layperson (optionally a patient) that the layperson has chosen a time from the healthcare professional's period of availability via mechanisms associated with the system as described herein. The notification optionally takes place via e-mail, phone, SMS, social media, within the graphical user interface as described herein, or by another communication medium. In association with such teaching, it is also therefore a step of the method of use of the telemedicine system to engage in notifying the patient and the healthcare professional associated of the mutually selected appointment time 2063.

In association with the choice of a time from a healthcare professional's period of availability, the system facilitates transfer of electronic personal health information, optionally including results from one or more diagnostic tests specific to the patient who scheduled an appointment with the healthcare professional. Such delivery optionally takes place via e-mail, the provision of delegated access to a Communicatively Connected Storage System, via the Healthcare Professional-Facing Application, by direct transfer to the healthcare professional's electronic medical records system, or by another mechanism. In association with such teaching, it is also therefore a step of the method of use of the telemedicine system to engage in delivering the results associated with one or more diagnostic tests that have evaluated the patient and the patient's demographic information in an interoperable format to the healthcare professional associated with the chosen appointment time 2064.

In association with the scheduled appointment between the healthcare professional and the patient, it is a further teaching of the system to facilitate communication between the healthcare professional and the patient. In an embodiment, the communication takes place via a videoconferencing system configured to take place within both the Healthcare Professional-Facing Application and the Patient-Facing Application, facilitated within the Healthcare Professional-Facing Application for the healthcare professional and within the Patient-Facing Application for the patient. In an embodiment, the videoconferencing system consists of another separate videoconferencing application, optionally the Skype, VeeSee or Zoom applications. In one example, a notification generated by the system contains a hyperlink configured to trigger the videoconferencing mechanism at the time of the scheduled appointment. In various embodiments, the videoconferencing is encrypted to accomplish compliance with relevant regulations such as HIPAA. In an alternative embodiment, the communication between the healthcare provider and the patient is a phone call. In one embodiment, the phone call is configured as a VOIP call facilitated within the Healthcare Professional-Facing Application for the healthcare professional and within the Patient-Facing Application for the patient. In association with such teaching, it is also therefore a step of the method of use of the telemedicine system to engage in facilitating the communication between the patient and the healthcare professional associated with the chosen appointment time at the chosen appointment time 2065.

It is a teaching of an embodiment to optionally trigger an appointment following an undesirable or suboptimal result indicated on a diagnostic test. For instance, in an example, receiving a negative result on a diagnostic test configured to evaluate for the presence or absence of PdG during the time period inclusive of 7-10 days past suspected ovulation constitutes one example of an undesirable or suboptimal result. In various examples, the results during a period within one period within a menstrual cycle can be used in identifying a trend if similar results are observed during the same or similar period one menstrual cycle to the next or to subsequent menstrual cycles, such periods optionally measured from menstrual cycle to menstrual cycle in days past ovulation or days past suspected ovulation. It is a teaching of the system to suggest a telemedicine consultation in association with the telemedicine system as described herein following the identification of a trend meriting such consultation. In accordance, a method of use of the system further comprises the step of suggesting a telemedicine consultation with a healthcare professional following the identification of one or more trends associated with the undesirable absence of at least one hormone or hormonal analyte during a specified timeframe 2017. Optionally, as described above, it is a teaching of the system and exemplary method of use that the one or more trends associated with such step comprises the undesirable absence of pregnanediol glucuronide as indicated by the stored results associated with any of the dates occurring from 7-10 days past the subject woman's ovulation date over a plurality of that subject woman's menstrual cycles 2018.

In another instance, failing to receive an indication of a positive result for the presence of PdG by a diagnostic test as described herein during the entirety of a woman's single menstrual cycle constitutes another example of an undesirable result. In association with such teaching, it is also therefore a step of the method of use of the telemedicine system to engage in generating a prompt to the patient via the Patient-Facing Application to schedule an appointment following at least one undesirable interpreted result of the diagnostic test 2066. It is likewise a teaching of various embodiments of the invention, including the system configured as a telemedicine system, to engage in identifying one or more trends associated with the undesirable absence of at least one hormone or hormonal analyte during a specified timeframe as indicated by a plurality of stored results each generated by a diagnostic test, optionally comprising a lateral flow assay test 2013.

The present inventor has recognized that the quantities of diagnostic tests needed to facilitate the deployment of the above system may be specific, therefore it is a teaching of an embodiment to provide specific quantities of various configurations of diagnostic tests as described elsewhere herein. The present inventor has recognized that for many, it is convenient and easy to download the Patient-Facing Application configured to enable such telemedicine system in association with the specified quantities of diagnostic tests as described herein. It is therefore a teaching of an embodiment of the system described herein to package the diagnostic tests in the specified quantities as described elsewhere herein into a single container additionally comprising instructions for utilization of the Patient-Facing Application.

In association with various embodiments of the invention as described herein including the telemedicine system, the present inventor has recognized that it is advantageous to estimate and trigger the purchase and delivery of diagnostic tests on a recurring basis. In an example, the purchase and delivery takes place in association with a subscription. In one example, the system is configured with the specific quantity or quantities of diagnostic tests configured to evaluate a bodily fluid for the presence or absence of a plurality of hormones and analytes at a threshold, such hormones and analytes, such hormones and analytes including LH and PdG, included in a single container. In an embodiment, the single container comprises a quantity selected from the range inclusive of 4-15 of diagnostic tests configured to evaluate a bodily fluid for the presence or absence of PdG at a threshold. In an embodiment, the single container comprises a quantity selected from the range inclusive of 5-25 of diagnostic tests configured to evaluate a bodily fluid for the presence or absence of LH at a threshold. The present inventor has recognized the significance of these ranges in that the ranges correspond to the amount of such tests necessary for adequately testing a single menstrual cycle in the vast majority of women. In an embodiment, the single container comprises a quantity of diagnostic tests configured to evaluate a bodily fluid for the presence or absence of PdG at a threshold selected from the range inclusive of 4-15 diagnostic tests. In an example, the system is further configured to automatically trigger the purchase and delivery of an additional such container on a date not later than the date the user of the system would run out of diagnostic tests if the user consumed a diagnostic test once daily. In one example, the amount of diagnostic tests, regardless of the hormones and/or analytes tested, corresponds to the number of diagnostic tests required for use in one menstrual cycle as described elsewhere herein. In association with such teaching, it is also therefore a step of the method of use of various embodiments of the system including optionally the telemedicine system to engage in triggering a purchase and delivery of additional lateral flow assay tests following the usage of a quantity of lateral flow assay tests correlative to one menstrual cycle 2024.

This invention was made with United States Government support under Agreement No. FA8649-20-9-9107, awarded by the United States Government. The United States Government has certain rights in the invention.

The invention, including its methods of use, disclosed herein comprise one or more steps or actions for achieving the described method. The method steps and/or actions may be interchanged with one another without departing from the scope of the claims. The order and/or use of specific steps and/or actions may be modified without departing from the scope of the claims.

Skilled artisans will appreciate that elements in the figures are illustrated for simplicity and clarity and have not necessarily been drawn to scale. For example, the dimensions of some of the elements in the figures may be exaggerated relative to other elements to help improve understanding of various embodiments. In addition, the description and drawings do not necessarily require the order illustrated. It will be further appreciated that certain actions and/or steps may be described or depicted in a particular order of occurrence while those skilled in the art will understand that such specificity with respect to sequence is not actually required.

Apparatus and method components have been represented where appropriate by conventional symbols in the drawings, showing only those specific details that are pertinent to understanding the various embodiments so as not to obscure the disclosure with details that will be readily apparent to those of ordinary skill in the art having the benefit of the description herein. Thus, it will be appreciated that for simplicity and clarity of illustration, common and well-understood elements that are useful or necessary in a commercially feasible embodiment may not be depicted in order to facilitate a less obstructed view of these various embodiments.

In the foregoing specification, specific embodiments have been described. However, one of ordinary skill in the art appreciates that various modifications and changes can be made without departing from the scope of the disclosure as set forth in the claims to follow in a subsequent disclosure. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all subsequent claims.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The terms “coupled,” “connected” and “linked” as used herein is defined as connected, although not necessarily directly and not necessarily mechanically. A device or structure that is “configured” in a certain way is configured in at least that way, but may also be configured in ways that are not listed. Also, the sequence of steps in a flow diagram or elements in the claims, even when preceded by a letter does not imply or require that sequence. Any noun in the singular is also intended to encompass the noun in the plural and vice versa, unless specifically stated as otherwise intended. Any pronoun or other identifier in the female form is also intended to encompass the pronoun or other identifier in the male form and vice versa, unless specifically stated as otherwise intended.

While the foregoing is directed to aspects of the present disclosure, other and further aspects of the disclosure may be devised without departing from the basic scope thereof.

### INDUSTRIAL APPLICABILITY

The invention described herein has particular applicability in the healthcare industry. More specifically, the invention as described herein has specific applications in telemedicine applications, diagnostic testing applications, and fertility applications as described elsewhere herein.

1-52. (canceled)

53. A menstrual cycle tracking system, comprising:
one diagnostic test or a plurality of diagnostic tests each configured to evaluate a sample of bodily fluid from a subject woman collected on a daily basis during a menstrual cycle,
at least one of the one diagnostic test or a plurality of diagnostic tests comprising at least one testing zone and corresponding result indication line configured to detect for the presence or absence of pregnanediol glucuronide at a threshold selected from the range inclusive of 1 μg/mL-10 μg/mL; and
a computing device configured to collect one result or a plurality of results indicated by the one diagnostic test or the plurality of diagnostic tests and evaluate the one result or plurality of results to generate one interpretation or a plurality of interpretations following a result,
the computing device further configured to store the one interpretation or a plurality of interpretations and display the one interpretation or plurality of interpretations based on the one result or plurality of results to a graphical user interface.

54. The menstrual cycle tracking system of claim 53, the one interpretation or a plurality of interpretations selected from the group consisting of:
following a result of a 1.5-fold decrease in FSH within a single menstrual cycle, the interpretation comprising an indication that a follicle has been selected;
following a result of a 1.5-fold decrease in FSH within a single menstrual cycle, the interpretation comprising an indication of the fertile window opening and the appropriate time to engage in intercourse for conception;
following a result of a 1.5-fold decrease in FSH within a single menstrual cycle, or on the eighth day of the menstrual cycle, whichever occurs first, the interpretation comprising an indication that it is the appropriate time to commence testing for an estrogen metabolite, optionally E3G, and an instruction to commence testing for the estrogen metabolite, optionally E3G;
following a result of a 1.5-fold decrease in FSH within a single menstrual cycle, the interpretation comprising an indication that it is the appropriate time to discontinue testing for FSH and to commence testing for estrogen and an instruction to discontinue testing for FSH and to commence testing for estrogen;
following a result of a persistently high level of FSH, the interpretation comprising an indication of the likelihood of onset of menopause;
following a result of a persistently high level of FSH, the interpretation comprising an indication of the likelihood of that ovulation may not occur this cycle or a high risk of anovulation;
following a result of a 1.5 fold increase in an estrogen metabolite, optionally E3G, within a single menstrual cycle, an interpretation comprising an indication that a follicle has matured;
following a result of a 1.5 fold increase in an estrogen metabolite, optionally E3G, within a single menstrual cycle, an interpretation comprising an indication of the fertile window opening;
following a result of a 1.5 fold increase in an estrogen metabolite, optionally E3G, within a single menstrual cycle, an interpretation comprising an indication of the follicle secreting estrogen;
following a result of a 1.5 fold increase in an estrogen metabolite, optionally E3G, within a single menstrual cycle, an interpretation comprising an indication that it is the appropriate time to commence testing for LH and an instruction to commence testing for LH;
following a result of a 1.5 fold increase in an estrogen metabolite, optionally E3G, an interpretation comprising an indication that it is the start of the fertile window and the appropriate time to engage in intercourse for conception;
following a result of a persistently low level of an estrogen metabolite, optionally E3G, an indication that the subject woman will not ovulate during the menstrual cycle;
following a result of a persistently low level of an estrogen metabolite, optionally E3G, an indication that the subject woman is likely not fertile during the menstrual cycle;
following a result of the presence of LH at a threshold, an interpretation comprising an indication that ovulation is imminent;
following a result of the presence of LH at a threshold, an interpretation comprising an indication of elevated fertility or peak fertility;
following a result of the presence of LH at a threshold, an interpretation comprising an indication that the subject woman should engage in sexual intercourse to conceive;
following a result of the presence of LH at a threshold, an interpretation comprising an indication that the it is the appropriate time to commence testing for PdG and an instruction to commence testing for PdG;
following a result of the presence of LH at a threshold, an interpretation comprising an indication that the it is the appropriate time to commence testing for progesterone and an instruction to commence testing for progesterone;
following a result of a persistently low level of LH, an interpretation comprising an indication that ovulation may not occur during this menstrual cycle;
following a result of a persistently low level of LH, an interpretation comprising an indication of the likelihood that ovulation is insufficient in this menstrual cycle for the subject woman to conceive;
following a result of the presence of PdG at a threshold on the days inclusive of 7-10 days past ovulation, an interpretation comprising an indication that the subject woman has sufficiently ovulated;
following a result of the presence of PdG at a threshold, an interpretation comprising an indication that the infertile period has begun;
following a result of the presence of PdG at a threshold, an interpretation comprising an indication that the subject woman may engage in sexual intercourse with a low risk of conceiving until the onset of menstruation in the subsequent menstrual cycle;
following at least one result of the absence of PdG at a threshold on one of the days selected from the range inclusive of 7-10 days past ovulation, an interpretation the woman has not sufficiently ovulated;
following a result of the presence of hCG at a threshold, an interpretation comprising an indication of pregnancy;
following a result of the absence of hCG at a threshold, an interpretation comprising an indication that the subject woman is not pregnant; and
following a result of the presence of hCG at a threshold and a result of the absence of PdG at a threshold, an interpretation comprising an indication that the subject woman has likely not produced enough progesterone to sustain pregnancy and that the subject woman should receive progesterone supplementation.

55. The menstrual cycle tracking system of claim 53, the plurality of diagnostic tests comprising a plurality of testing zones and corresponding result indication lines each configured to evaluate for the presence or absence of a hormone or analyte selected from the group consisting of FSH, E3G, LH, hCG and PdG.

56. The menstrual cycle tracking system of claim 53, further comprising a patient-facing application,
the patient-facing application comprising a patient profile, an electronic personal health information exporter, and a graphical user interface;
the patient-facing information configured to operate in association with a computing device and communicatively connected storage medium; and
the patient-facing application configured to record and store the result indicated on at least a diagnostic test configured to detect for the presence or absence of pregnanediol glucuronide at a threshold.

57. The menstrual cycle tracking system of claim 53, further comprising a healthcare professional-facing application,
the healthcare professional-facing application comprising a healthcare profile, a ePHI importer/exporter and a graphical user interface.

58. The menstrual cycle tracking system of claim 53, further comprising a calendar configured to display the result or interpretation associated with each diagnostic test in a graphical user interface in association with the date and time each diagnostic test was performed.

59. The menstrual cycle tracking system of claim 53, further comprising a fertility tracking system comprising instructions for the sequence and timing to perform the diagnostic tests, the instructions delivered via a graphical user interface operated in association with the computing device.

60. The menstrual cycle tracking system of claim 53, further comprising a telemedicine system configured to facilitate the scheduling and conduct of a telemedicine appointment with a healthcare professional for further evaluation of the plurality of diagnostic tests.

61. The menstrual cycle tracking system of claim 53, further comprising a seed consumption system, configured to prompt a user to consume specified seeds based on the result indicated on each of the plurality of diagnostic tests.

62. The menstrual cycle tracking system of claim 53, further comprising a predicting fertile window system configured to provide a notification that the fertile period has begun following an indication on a diagnostic test performed during the menstrual cycle that LH is present at a threshold or an indication on a diagnostic test performed during the menstrual cycle that FSH and/or E3G is present at a threshold, and configured to provide a notification that the fertile period has ended following an indication on a diagnostic test performed during the menstrual cycle that PdG is present at a threshold.

63. A fertility tracking system, comprising:
a plurality of diagnostic tests each consisting of a lateral flow assay, each lateral flow assay comprising a testing zone and corresponding result indication line configured to detect for the presence or absence of pregnanediol glucuronide in urine at a threshold selected from the range of 1 μg/mL-10 μg/mL and a control line, configured such that only one perceptible line observable on the diagnostic test indicates a positive result and the presence of two perceptible lines observable on the diagnostic test indicates a negative result for the presence of pregnanediol glucuronide at a threshold selected from the range inclusive of 1 μg/mL-10 μg/mL.

64. A fertility tracking system for evaluating a menstrual cycle, comprising:
a plurality, of a quantity selected from the range inclusive of 4-15, of diagnostic tests each configured to evaluate for the presence or absence of pregnanediol glucuronide at a threshold selected from the range inclusive of 1 μg/mL-10 μg/mL in an applied fluid; and
a plurality, of a quantity selected from the range inclusive of 7-25, of diagnostic tests each configured to evaluate for the presence or absence of luteinizing hormone at a threshold selected from the range inclusive of 15 mIU/mL-50 mIU/mL in an applied fluid.

65. The fertility tracking system of claim 64, further comprising a plurality, at a quantity selected from the range inclusive of 7-25, of diagnostic tests configured to evaluate for the presence of FSH in urine.

66. The fertility tracking system of claim 64, further comprising a plurality, of a quantity selected from the range inclusive of 7-25, of diagnostic tests configured to evaluate for the presence of E3G in urine.

67. The fertility tracking system of claim 64, additionally comprising instructions for utilization, comprising:
an instruction to commence testing for FSH, by utilizing a single diagnostic test from a plurality of diagnostic tests each configured to evaluate for the presence of FSH on a date selected from the range of 2-3 days following the onset of menstruation;
an instruction to commence testing for E3G by utilizing a single diagnostic test from a plurality of diagnostic tests each configured to evaluate for the presence of E3G following at least a 1.5-fold decrease from one diagnostic test configured to evaluate for the presence of FSH to another diagnostic test configured to evaluate for the presence of FSH performed on a different day in the same menstrual cycle;
an instruction to commence testing for LH by utilizing a single diagnostic test from a plurality of diagnostic tests each configured to evaluate for the presence or absence of LH at a threshold following at least an 1.5 fold increase from one diagnostic test configured to evaluate for the presence of E3G to another diagnostic test configured to evaluate for the presence of E3G performed on a different day in the same menstrual cycle; and
an instruction to commence testing for PdG by utilizing a single diagnostic test from a plurality of diagnostic tests each configured to evaluate for the presence of PdG at a threshold following a positive result for LH on a diagnostic test obtained within the same menstrual cycle.

68. The fertility tracking system of claim 67, the instructions for utilization formatted for digital delivery via a computing device or a digital reader.

69. The fertility tracking system of claim 64, further comprising a single container to enclose the plurality of diagnostic tests together.

70. The fertility tracking system of claim 64, further comprising at least one snack bar comprising pumpkin seeds and flax seeds and at least one snack bar comprising sesame seeds and sunflower seeds, and a daily recurring instruction following menstruation to consume pumpkin seeds and flax seeds once daily until the occurrence of a diagnostic test result indicating the presence of LH at a threshold on any day during the cycle, and following the occurrence of a diagnostic test result indicating the presence of LH at a threshold on any day during the cycle, a daily recurring instruction to consume sesame seeds and sunflower seeds once daily until the onset of menstruation.

71. The fertility tracking system of claim 70, each daily recurring instruction delivered via the graphical user interface associated with an application operating on a smartphone.

72. The fertility tracking system of claim 70, each daily recurring instruction delivered via a display integrated into a cartridge also containing a diagnostic test.

73. The fertility tracking system of claim 70, each daily recurring instruction delivered via a display integrated into a digital reader.

74. The fertility tracking system of claim 64, further comprising a plurality of progesterone supplement doses, each formulated in an oil suspension.

75. The fertility tracking system of claim 64, further comprising an instruction to consume the plurality of progesterone supplement doses during the subsequent menstrual cycle following an indication of the absence of PdG at a threshold in the applied fluid on any diagnostic test taken during the timeframe of 7-10 days past ovulation.

76. The fertility tracking system of claim 75, the instruction to ingest the progesterone supplement following an indication on a diagnostic test of the absence of PdG at a threshold in an applied fluid delivered in association with an application operating on a smartphone.

77. A fertility tracking system for evaluating a menstrual cycle, comprising:
a plurality, of a quantity selected from the range inclusive of 10-25, of diagnostic tests, each diagnostic test configured to evaluate an applied fluid for at least the presence or absence of pregnanediol glucuronide at a threshold selected from the range inclusive of 1 μg/mL-10 μg/mL in a first testing zone and the presence or absence of luteinizing hormone at a threshold selected from the range inclusive of 15 mIU/mL-50 mIU/mL in a second testing zone.

78. The fertility tracking system of claim 77, further comprising a diagnostic test key configured to map the color intensity of an indicated result to a concentration of a hormone or analyte correlating to the color intensity.

79. The fertility tracking system of claim 77, each diagnostic test configured to additionally evaluate for the concentration of FSH in a testing zone and provide a result comprising a color intensity, the color intensity correlating to a concentration of FSH in the applied fluid.

80. The fertility tracking system of claim 77, each diagnostic test configured to additionally evaluate for the concentration of E3G in a testing zone and provide a result comprising a color intensity, the color intensity correlating to a concentration of E3G in the applied fluid.

81. The fertility tracking system of claim 77, further comprising a single container configured to enclose the plurality of diagnostic tests, each diagnostic test placed individually into a sealed packet, together.

82. The fertility tracking system of claim 77, further comprising at least one single consumable food item comprising pumpkin seeds in an amount selected from the range inclusive of 6.5 grams-16 grams and flax seeds in an amount selected from the range inclusive of 6.5 grams-16 grams, and at least one single consumable food item comprising sesame seeds in an amount selected from the range inclusive of 6.5 grams-16 grams and sunflower seeds in an amount selected from the range inclusive of 6.5 grams-16 grams, and commencing upon the occurrence of menstruation a daily recurring instruction to consume pumpkin seeds and flax seeds once daily, the daily recurring instruction to consume pumpkin seeds and flax seeds once daily terminating upon the occurrence of a diagnostic test result indicating the presence of LH at a threshold on any day during the cycle, and following the occurrence of a diagnostic test result indicating the presence of LH at a threshold on any day during the cycle, a daily recurring instruction to consume sesame seeds and sunflower seeds once daily, the daily recurring instruction to consume sesame seeds and sunflower seeds once daily terminating upon the onset of menstruation.

83. The fertility tracking system of claim 82, each daily recurring instruction delivered via the graphical user interface associated with an application operating on a smartphone.

84. The fertility tracking system of claim 82, each daily recurring instruction delivered via a display integrated into a cartridge also containing a diagnostic test.

85. The fertility tracking system of claim 82, each daily recurring instruction delivered via a display integrated into a digital reader.
